(19) United States
(12) Patent Application Publication
(10) Pub. No.: US 2023/0150978 A1
(43) Pub. Date: May 18, 2023

(54) QUINAZOLINONE DERIVATIVES AND USES THEREOF FOR TREATING A CANCER

(71) Applicant: ENYO PHARMA, LYON (FR)

(72) Inventors: LAURÈNE MEYNIEL-SCHICKLIN, LYON (FR); PETER MACHIN, LONDON (GB); ERIC MELDRUM, RIEHEN (CH); BENOÎT DE CHASSEY, LYON (FR); FABRICE GUILLIER, BELLENEUVE (FR); CHRISTINE MASSARDIER, DIJON (FR); JÉRÔME AMAUDRUT, DIJON (FR); CHRISTIAN MONTALBETTI, POMEZIA (IT); DAVID COUSIN, NOTTINGHAM, NOTTINGHAMSHIRE (GB); JEAN-LAURENT PAPARIN, FERNEY VOLTAIRE (FR)

(21) Appl. No.: 17/915,118

(22) PCT Filed: Mar. 29, 2021
(86) PCT No.: PCT/EP2021/058176

§ 371 Date: Sep. 28, 2022

(30) Foreign Application Priority Data
Mar. 30, 2020 (WO) 20166842.3

(51) Int. Cl. C07D 403/06; C07D 413/06; C07D 401/06; C07D 471/04; C07D 403/14; C07D 491/056; C07D 401/14; A61P 35/00

(52) U.S. Cl. CPC C07D 403/06; C07D 413/06; C07D 401/06; C07D 471/04; C07D 403/14; C07D 491/056; C07D 401/14; A61P 35/00

(57) ABSTRACT

The present invention relates to a new class of quinazolinone derivatives of formula (I), and their uses for treating a cancer. The present invention further relates to pharmaceutical compositions comprising compounds of formula (I).

### FIELD OF THE INVENTION

The present invention relates to the field of medicine, in particular quinazolinone derivatives and their uses for treating a cancer.

### BACKGROUND OF THE INVENTION

Cancer is the second leading cause of death worldwide. Indeed, nearly 1 in 6 deaths is due to cancer. The prevalence of cancer is also extremely high as more than 15 million new cases are diagnosed each year, and the number of new cases is expected to rise by about 70% over the next 2 decades. Among the most common cancers, lung cancers account for 1.69 million deaths per year, colorectal cancer for 774 000 deaths per year, and breast cancer for 571 000 deaths per year.

Many treatment options exist nowadays for cancer, including for example surgery, chemotherapy, radiation therapy, hormonal therapy, targeted therapy and palliative care. The choice of the best treatment depends on the type, location and grade of the cancer as well as the patient's health and preferences. However, there is still an important proportion of cancers, especially in late stage and/or metastatic cancers that remains resistant to treatment.

In the last few decades immunotherapy has become an important part of cancer treatment strategies. Cancer immunotherapy relies on the use of the immune system to treat cancer. Among the diversity of immunotherapy treatments that have been developed over time, immune checkpoint inhibitor therapies are particularly promising. However, the response to immune checkpoint inhibitors can be low.

A novel approach for treating cancer is to use immunogenic cell death (ICD). ICD is defined by chronic exposure of damage-associated molecular patterns (DAMPs) in the tumour microenvironment (TME), which stimulates the dysfunctional antitumour immune system. Important DAMPs include the exposure of CALR, PDIA3, HSP70 and HSP90 on the membrane surface and the release of HMGB1 and ATP from the dying cells. Chronic exposure of damage-associated molecular patterns (DAMPs) attracts receptors and ligands on dendritic cells (DCs) and activates immature DCs to transition to a mature phenotype, which promotes the processing of phagocytic cargo in DCs and accelerates the engulfment of antigenic components by DCs. Consequently, via antigen presentation, DCs stimulate specific T cell responses that kill more cancer cells. The induction of ICD contributes to long-lasting protective antitumour immunity. Therefore, ICD induction has emerged as novel cancer therapies.

Some classic ICD inducers, such as anthracyclines (doxorubicin, idarubicin and mitoxantrone), all the chemical PP1/GADD34 inhibitors (tautomycin, calyculin A and salubrinal), cardiac glycosides (CGs, digoxin, digitoxin, ouabain and lanatoside C), oxaliplatin, bleomycin (BLM, an antitumour antibiotic glycopeptide), cyclophosphamide (CTX), and shikonin (SK), have been widely studied.

However, there remains a need to identify further agents that can promote ICD.

### SUMMARY OF THE INVENTION

In this context, the inventors have provided new quinazolinone derivatives exhibiting an activity of ICD inducer, demonstrating thereby the therapeutic interest of such compounds in medicine, more particularly in cancer therapies.

The present invention thus provides a compound having the following formula (I):

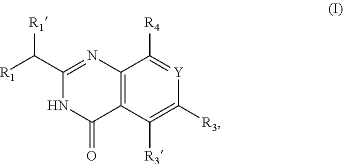

wherein:
R₁ represents a fused arylheterocycloalkyl selected in the group consisting of:
   an indolinyl,
   an isoindolinyl,
   a 1,2,3,4-tetrahydroquinolinyl,
   a 1,2,3,4-tetrahydroisoquinolinyl,
   a 2,3-dihydroquinoxalinyl,
   a 3,4-dihydro-1,4-benzoxazinyl,
   a 2,3,4,5-tetrahydro-1,5-benzodiazepinyl
   a 2,3,4,5-tetrahydro-1-benzazepinyl, and
   a 2,3,4,5-tetrahydro-1,5-benzoxazepinyl,
said fused arylheterocycloalkyl is optionally substituted by at least one radical selected in the group consisting of:
   a halogen,
   a (C₁-C₆)alkyl optionally substituted by at least one hydroxy, an amino or a halogen,
   a (C₁-C₆)alkyloxy optionally substituted by at least one hydroxy, an amino or a halogen,
   a cyano,
   a cycloalkyl or a spirocycloalkyl,
   a hydroxy,
   a —NH—SO₂(CH₃),
   a —NR₅R₆ with R₅ and R₆ being independently a hydrogen, a (C₁-C₆)alkyl or a —C(O)R₈ with R₅ being a (C₁-C₆)alkyl, and
R_{1′} represents a hydrogen or a (C₁-C₆)alkyl;
Y represents:
   a nitrogen, or
   a —CR₂ group in which R₂ is a radical selected in the group consisting of a hydrogen, a halogen, a (C₁-C₆)alkyl optionally substituted by at least one halogen, and a (C₁-C₆)alkyloxy optionally substituted by at least one halogen;
R₃ and R_{3′} represents independently a radical selected in the group consisting of:
   a 3-10 membered ring selected in the group consisting of a cycloalkyl, a heterocycloalkyl, and an aryl, said 3-10 membered ring is optionally substituted by a radical selected in a group consisting of:
      a (C₁-C₆)alkyl optionally substituted by a (C₁-C₆)alkyloxy, a hydroxy, a halogen, or a —NR₅R₆ with R₅ and R₆ being independently a hydrogen,
         a (C₁-C₆)alkyloxy optionally substituted by a a halogen,
         a halogen, and
         a hydroxy,
   a (C₁-C₆)alkyl and a (C₁-C₆)alkyloxy optionally substituted by a halogen, a (C₁-C₆)alkyloxy or a —NH—CO₂—R₇ with R₇ being a (C₁-C₆)alkyl,
   a —NR₅R₆ with R₅ and R₆ being independently a hydrogen or a (C₁-C₆)alkyl,
   a halogen,
   a hydrogen, and
   a cyano or
R₃ may form with R₂ a dioxolanyl; and
R₄ represents a hydrogen or a (C₁-C₆)alkyloxy;
   and the stereoisomers, the tautomers, and the pharmaceutical salts thereof;
   with the proviso that the compound of formula (I) is not:
6-chloro-2-(indolin-1-ylmethyl)-3H-quinazolin-4-one;
6-chloro-2-(3,4-dihydro-2H-quinolin-1-ylmethyl)-3H-quinazolin-4-one;
6-chloro-2-(3,4-dihydro-1H-isoquinolin-2-ylmethyl)-3H-quinazolin-4-one;
6-chloro-2-[(4-hydroxy-5,8-dimethoxy-3,4-dihydro-1H-isoquinolin-2-yl)methyl]-3H-quinazolin-4-one;
2-(indolin-1-ylmethyl)-6,7-dimethoxy-3H-quinazolin-4-one; and
7-chloro-2-(indolin-1-ylmethyl)-3H-quinazolin-4-one.

In a preferred embodiment, Y represents a nitrogen or a —CR₂ group in which R₂ is a radical selected in the group consisting of a hydrogen, a halogen, preferably a fluorine or a chlorine, a (C₁-C₆)alkyloxy optionally substituted by a halogen, preferably a methoxy or a trifluoromethoxy, a (C₁-C₆)alkyl optionally substituted by a halogen, preferably a methyl, or a trifluoromethyl, more preferably R₂ is a hydrogen or a halogen, preferably a fluorine and a chlorine, and even more preferably R₂ is a hydrogen.

In a further preferred embodiment, R_{3′} represents a hydrogen.

In a further preferred embodiment, R₃ represents a radical selected in the group consisting of:
a 3-10 membered ring selected in the group consisting of a cyclopropyl, an azetidinyl, a pyrrolidinyl, a phenyl, and a piperidinyl, said 3-10 membered ring is optionally substituted by at least one radical selected in a group consisting of:
   a methyl substituted by a methoxy or a hydroxy,
   a (C₁-C₆)alkyloxy, preferably a methoxy, and
   a hydroxy,
a (C₁-C₆)alkyloxy, preferably a methoxy, a trifluromethoxy, an isopropyloxy, an ethoxy, a propoxy, or a (C₁-C₆)alkyloxy substituted by a methoxy or a —O—(CH₂)₄NHCO₂tBu,
a methyl, a trifluoromethyl, or an isopropyl,
a —NR₅R₆ with R₅ and R₆ being a methyl,
a chlorine or a flurorine, preferably a chlorine,
a hydrogen, and
a cyano.

More preferably, R₃ represents a radical selected in the group consisting of a cyclopropyl, a methoxy, a —NR₅R₆ with R₅ and R₆ being a methyl, an azetidinyl, an isopropyl, a chlorine, a pyrrolidine optionally substituted by a —CH₂—O—CH₃, and a methyl.

In a first aspect, R₁ represents an optionally substituted indolinyl or an optionally substituted isoindolinyl.

According to this first aspect, a preferred compound of formula (I) is such that:
R₁ represents an unsubstituted indolinyl, an indolinyl substituted by at least one radical selected in the group consisting of a halogen, preferably a fluorine, and a (C₁-C₆)alkyl, preferably a methyl or an isopropyl, or an indolinyl substituted by two (C₁-C₆)alkyl, preferably two methyls;
R_{1′} represents a hydrogen;
Y represents:
   a nitrogen, or
   a —CR₂ group in which R₂ is a radical selected in the group consisting of a hydrogen, and a halogen, preferably a fluorine or a chlorine;
R₃ represents a radical selected in the group consisting of:
   a cyclopropyl, an azetidinyl, a pyrrolidinyl optionally substituted by a —CH₂—O—CH₃,
   a (C₁-C₆)alkyloxy, preferably a methoxy, or a (C₁-C₆)alkyl, preferably a methyl, or an isopropyl,
   a —NR₅R₆ with R₅ and R₆ being a methyl,
   a (C₁-C₆)alkyl, preferably a methyl, and
   a halogen, preferably a chlorine;
R_{3′} represents a hydrogen; and
R₄ represents a hydrogen.

In a second aspect, R₁ represents an optionally substituted 1,2,3,4-tetrahydroquinolinyl. According to this second aspect, a preferred compound of formula (I) is such that:
R₁ represents an unsubstituted 1,2,3,4-tetrahydroquinolinyl or a 1,2,3,4-tetrahydroquinolinyl substituted by at least one radical selected in the group consisting of a halogen, preferably a fluorine or a chlorine, a (C₁-C₆)alkyl, preferably a methyl, and a (C₁-C₆)alkyloxy, preferably a methoxy;
R_{1′} represents a hydrogen;
Y represents a —CR₂ group in which R₂ is a hydrogen;
R₃ represents a radical selected in the group consisting of a (C₁-C₆)alkyloxy, preferably a methoxy, and a halogen, preferably a chlorine;
R_{3′} represents a hydrogen; and
R₄ represents a hydrogen.

In a third aspect, R₁ represents an optionally substituted 1,2,3,4-tetrahydroisoquinolinyl. According to this third aspect, a preferred compound of formula (I) is such that:
R₁ represents a 1,2,3,4-tetrahydroisoquinolinyl substituted by at least one radical selected in the group consisting of a cyano, a hydroxy, a (C₁-C₆)alkyloxy, preferably a methoxy, and a (C₁-C₆)alkyl, preferably a methyl, or a 1,2,3,4-tetrahydroisoquinolinyl substituted by one hydroxy and two (C₁-C₆)alkyloxy, preferably two methoxy.
R_{1′} represents a hydrogen;
Y represents a —CR₂ group in which R₂ is a hydrogen;
R₃ represents a radical selected in the group consisting of a (C₁-C₆)alkyloxy, preferably a methoxy, and a halogen, preferably a chlorine;
R_{3′} represents a hydrogen; and
R₄ represents a hydrogen.

In a fourth aspect, R₁ represents an optionally substituted 2,3,4,5-tetrahydro-1,5-benzodiazepinyl or an optionally substituted 2,3,4,5-tetrahydro-1-benzazepinyl.

According to this fourth aspect, a preferred compound of formula (I) is such that:
R₁ represents an unsubstituted 2,3,4,5-tetrahydro-1-benzazepinyl, or a 2,3,4,5-tetrahydro-1,5-benzodiazepinyl substituted by at least one (C₁-C₆)alkyl, preferably a methyl,
R_{1′} represents a hydrogen;
Y represents a —CR₂ group in which R₂ is a hydrogen;
R₃ represents a radical selected in the group consisting of a (C₁-C₆)alkyloxy, preferably a methoxy, and a halogen, preferably a chlorine;
R_{3Y} represents a hydrogen; and
R₄ represents a hydrogen.

In a more preferred embodiment, a compound of formula (I) is selected in the group consisting of:
Example #99. 6-cyclopropyl-2-(indolin-1-ylmethyl)-3H-quinazolin-4-one;
Example #11. 2-[(8-fluoro-3,4-dihydro-2H-quinolin-1-yl)methyl]-6-methoxy-3H-quinazolin-4-one;
Example #92. 6-(dimethylamino)-2-[(3,3-dimethylindolin-1-yl)methyl]-3H-pyrido[3,4-d]pyrimidin-4-one;
Example #77. 2-[(3,3-dimethylindolin-1-yl)methyl]-7-fluoro-6-methoxy-3H-quinazolin-4-one;
Example #93. 7-chloro-2-[(3,3-dimethylindolin-1-yl)methyl]-6-methoxy-3H-quinazolin-4-one;
Example #2. 2-[(3,3-dimethylindolin-1-yl)methyl]-6-methoxy-3H-quinazolin-4-one;
Example #4. 6-methoxy-2-[(3-methylindolin-1-yl)methyl]-3H-quinazolin-4-one, enantiomer 1;
Example #74. 6-(dimethylamino)-2-[(3,3-dimethylindolin-1-yl)methyl]-3H-quinazolin-4-one;
Example #6. 2-[(3-isopropylindolin-1-yl)methyl]-6-methoxy-3H-quinazolin-4-one;
Example #80. 6-(dimethylamino)-7-fluoro-2-(indolin-1-ylmethyl)-3H-quinazolin-4-one;
Example #100. 6-(azetidin-1-yl)-2-(indolin-1-ylmethyl)-3H-quinazolin-4-one;
Example #3. 6-methoxy-2-[(3-methylindolin-1-yl)methyl]-3H-quinazolin-4-one;
Example #1. 2-(indolin-1-ylmethyl)-6-methoxy-3H-quinazolin-4-one;
Example #73. 6-(dimethylamino)-2-(indolin-1-ylmethyl)-3H-quinazolin-4-one;
Example #81. 2-(indolin-1-ylmethyl)-6-isopropyl-3H-quinazolin-4-one;
Example #7. 2-[(6-fluoroindolin-1-yl)methyl]-6-methoxy-3H-quinazolin-4-one;
Example #22. 6-methoxy-2-(2,3,4,5-tetrahydro-1-benzazepin-1-ylmethyl)-3H-quinazolin-4-one;
Example #76. 7-fluoro-2-(indolin-1-ylmethyl)-6-methoxy-3H-quinazolin-4-one;
Example #38. 6-chloro-2-[(3-methylindolin-1-yl)methyl]-3H-quinazolin-4-one enantiomer 1;
Example #12. 2-[(8-chloro-3,4-dihydro-2H-quinolin-1-yl)methyl]-6-methoxy-3H-quinazolin-4-one;
Example #13. 6-methoxy-2-[(8-methoxy-3,4-dihydro-2H-quinolin-1-yl)methyl]-3H-quinazolin-4-one;
Example #79. 2-[(3,3-dimethylindolin-1-yl)methyl]-6-methoxy-3H-pyrido[3,4-d]pyrimidin-4-one;
Example #103. 2-(indolin-1-ylmethyl)-6-[3-(methoxymethyl)pyrrolidin-1-yl]-3H-quinazolin-4-one;
Example #62. 6-chloro-2-(2,3,4,5-tetrahydro-1-benzazepin-1-ylmethyl)-3H-quinazolin-4-one;
Example #43. 6-chloro-2-[(4-methylindolin-1-yl)methyl]-3H-quinazolin-4-one;
Example #82. 2-(indolin-1-ylmethyl)-6-(trifluoromethoxy)-3H-quinazolin-4-one;
Example #40. 6-chloro-2-[(3,3-dimethylindolin-1-yl)methyl]-3H-quinazolin-4-one;
Example #44. 6-chloro-2-[(7-methylindolin-1-yl)methyl]-3H-quinazolin-4-one;
Example #52. 6-chloro-2-[(5-methoxy-3,4-dihydro-2H-quinolin-1-yl)methyl]-3H-quinazolin-4-one;
Example #102. 2-(indolin-1-ylmethyl)-6-pyrrolidin-1-yl-3H-quinazolin-4-one;
Example #23. 6-methoxy-2-[(1-methyl-3,4-dihydro-2H-1,5-benzodiazepin-5-yl)methyl]-3H-quinazolin-4-one;
Example #14. 6-methoxy-2-[(5-methoxy-2,3-dihydro-1,4-benzoxazin-4-yl)methyl]-3H-quinazolin-4-one;
Example #53. 6-chloro-2-[(5-methyl-3,4-dihydro-2H-quinolin-1-yl)methyl]-3H-quinazolin-4-one;
Example #84. 2-(indolin-1-ylmethyl)-6-methyl-3H-quinazolin-4-one;
Example #16. 2-[(6-methoxy-4-oxo-3H-quinazolin-2-yl)methyl]-3,4-dihydro-1H-isoquinoline-5-carbonitrile;
Example #54. 6-chloro-2-[(8-methoxy-3,4-dihydro-2H-quinolin-1-yl)methyl]-3H-quinazolin-4-one;
Example #37. 6-chloro-2-[(3-methylindolin-1-yl)methyl]-3H-quinazolin-4-one;
Example #78. 2-(indolin-1-ylmethyl)-6-methoxy-3H-pyrido[3,4-d]pyrimidin-4-one;
Example #15. 2-(3,4-dihydro-2H-quinolin-1-ylmethyl)-6-methoxy-3H-quinazolin-4-one;
Example #5. 6-methoxy-2-[(3-methylindolin-1-yl)methyl]-3H-quinazolin-4-one, enantiomer 2;
Example #104. 6-[3-(hydroxymethyl)pyrrolidin-1-yl]-2-(indolin-1-ylmethyl)-3H-quinazolin-4-one;
Example #42. 6-chloro-2-(spiro[cyclopentane-1,3′-indoline]-1′-ylmethyl)-3H-quinazolin-4-one;
Example #85. 2-(indolin-1-ylmethyl)-6-(trifluoromethyl)-3H-quinazolin-4-one;
Example #86. 2-(indolin-1-ylmethyl)-7-methoxy-3H-quinazolin-4-one;
Example #87. 2-(indolin-1-ylmethyl)-6-isopropoxy-3H-quinazolin-4-one;
Example #47. 6-chloro-2-[(5-fluoroindolin-1-yl)methyl]-311-quinazolin-4-one (;
Example #63. 6-chloro-2-[(1-methyl-3,4-dihydro-2H-1,5-benzodiazepin-5-yl)methyl]-3H-quinazolin-4-one;
Example #45. 6-chloro-2-[(6-methylindolin-1-yl)methyl]-3H-quinazolin-4-one;
Example #88. 6-fluoro-2-(indolin-1-ylmethyl)-3H-quinazolin-4-one;
Example #58. 6-chloro-2-[(5-methoxy-3,4-dihydro-1H-isoquinolin-2-yl)methyl]-3H-quinazolin-4-one;
Example #59A. 6-chloro-2-(1,2,3,4-tetrahydroisoquinolin-2-ium-2-ylmethyl)-3H-quinazolin-4-one chloride;
Example #64. 6-chloro-2-(3,4-dihydro-2H-1,5-benzoxazepin-5-ylmethyl)-3H-quinazolin-4-one;
Example #46. 6-chloro-2-[(5-methylindolin-1-yl)methyl]-3H-quinazolin-4-one;
Example #94. 2-[(3,3-dimethylindolin-1-yl)methyl]-6,8-dimethoxy-3H-quinazolin-4-one;
Example #48. 2-[(5-bromoindolin-1-yl)methyl]-6-chloro-3H-quinazolin-4-one;
Example #17. 6-methoxy-2-[(5-methoxy-3,4-dihydro-1H-isoquinolin-2-yl)methyl]-3H-quinazolin-4-one;
Example #10. 2-(isoindolin-2-ylmethyl)-6-methoxy-3H-quinazolin-4-one;
Example #119. 6-[(3,3-dimethylindolin-1-yl)methyl]-7H-[1,3]dioxolo[4,5-g]quinazolin-8-one;
Example #97. 2-[(3,3-dimethylindolin-1-yl)methyl]-6-(2-methoxyethoxy)-3H-quinazolin-4-one;
Example #120. 6-chloro-2-(1-indolin-1-ylethyl)-3H-quinazolin-4-one;
Example #8. 2-[(7-fluoroindolin-1-yl)methyl]-6-methoxy-3H-quinazolin-4-one;
Example #90. tert-butyl N-[4-[[2-(indolin-1-ylmethyl)-4-oxo-3H-quinazolin-6-yl]oxy]butyl]carbamate;
Example #60. 6-chloro-2-[(7-methyl-3,4-dihydro-1H-isoquinolin-2-yl)methyl]-3H-quinazolin-4-one;
Example #18. 2-[(5-bromo-3,4-dihydro-1H-isoquinolin-2-yl)methyl]-6-methoxy-3H-quinazolin-4-one;
Example #41. 6-chloro-2-[(2-methylindolin-1-yl)methyl]-3H-quinazolin-4-one;
Example #91. 2-(indolin-1-ylmethyl)-7-(trifluoromethyl)-3H-quinazolin-4-one;
Example #19. 6-methoxy-2-[(8-methoxy-3,4-dihydro-1H-isoquinolin-2-yl)methyl]-3H-quinazolin-4-one;
Example #101. 2-(indolin-1-ylmethyl)-6-(3-methoxyazetidin-1-yl)-3H-quinazolin-4-one;
Example #39. 6-chloro-2-[(3-methylindolin-1-yl)methyl]-3H-quinazolin-4-one enantiomer 2;
Example #96. 2-[(3,3-dimethylindolin-1-yl)methyl]-6-(3-methoxypropoxy)-3H-quinazolin-4-one;
Example #20. 2-[(5-chloro-3,4-dihydro-1H-isoquinolin-2-yl)methyl]-6-methoxy-3H-quinazolin-4-one;
Example #55. 6-chloro-2-[(7-methoxy-3,4-dihydro-2H-quinolin-1-yl)methyl]-3H-quinazolin-4-one;
Example #106. 2-(indolin-1-ylmethyl)-6-(1-piperidyl)-3H-quinazolin-4-one;
Example #21. N-[2-[(6-methoxy-4-oxo-3H-quinazolin-2-yl)methyl]-3,4-dihydro-1H-isoquinolin-5-yl]methanesulfonamide;
Example #49. 6-chloro-2-[(7-methoxyindolin-1-yl)methyl]-3H-quinazolin-4-one;
Example #95. 2-[(3,3-dimethylindolin-1-yl)methyl]-4-oxo-3H-quinazoline-6-carbonitrile;
Example #50. 6-chloro-2-(isoindolin-2-ylmethyl)-3H-quinazolin-4-one;
Example #105. 6-(3-hydroxypyrrolidin-1-yl)-2-(indolin-1-ylmethyl)-3H-quinazolin-4-one;
Example #122. 2-(3,4-dihydro-1H-isoquinolin-2-ylmethyl)-6-methoxy-3H-quinazolin-4-one;
Example #123. 2-[(4-hydroxy-3,4-dihydro-1H-isoquinolin-2-yl)methyl]-6-methoxy-3H-quinazolin-4-one;
Example #128. 6-chloro-2-[(4-methyl-2,3-dihydroquinoxalin-1-yl)methyl]-3H-quinazolin-4-one;
Example #129. 2-[[3-(2-hydroxyethyl)indolin-1-yl]methyl]-6-methoxy-3H-quinazolin-4-one;
Example #127. 6-chloro-2-[(7-methyl-3,4-dihydro-2H-quinolin-1-yl)methyl]-3H-quinazolin-4-one;
Example #124. 2-[(5-fluoro-3,4-dihydro-1H-isoquinolin-2-yl)methyl]-6-methoxy-3H-quinazolin-4-one;
Example #133. 2-[(5-amino-3,4-dihydro-1H-isoquinolin-2-yl)methyl]-6-methoxy-3H-quinazolin-4-one;
Example #132. 2-(indolin-1-ylmethyl)-6-(4-methoxy-1-piperidyl)-3H-quinazolin-4-one;
Example #131. 2-[1-[(6-methoxy-4-oxo-3H-quinazolin-2-yl)methyl]indolin-3-yl]ethylammonium chloride;
Example #138. 7-fluoro-2-(indolin-1-ylmethyl)-3H-quinazolin-4-one;
Example #144. 6-chloro-2-((5-methoxyindolin-1-yl)methyl)quinazolin-4(3H)-one;
Example #130. 6-chloro-2-[(8-methoxy-3,4-dihydro-1H-isoquinolin-2-yl)methyl]-3H-quinazolin-4-one;
Example #125. 6-methoxy-2-[[5-(trifluoromethyl)-3,4-dihydro-1H-isoquinolin-2-yl]methyl]-3H-quinazolin-4-one;
Example #126. 2-[(4-fluoroindolin-1-yl)methyl]-6-methoxy-3H-quinazolin-4-one;
Example #9. 2-[[3-(hydroxymethyl)indolin-1-yl]methyl]-6-methoxy-3H-quinazolin-4-one; and
Example #57. 6-chloro-2-(2,3-dihydro-1,4-benzoxazin-4-ylmethyl)-3H-quinazolin-4-one.

In an even more preferred embodiment, a compound of formula (I) is selected in the group consisting of:
Example #99. 6-cyclopropyl-2-(indolin-1-ylmethyl)-3H-quinazolin-4-one;
Example #11. 2-[(8-fluoro-3,4-dihydro-2H-quinolin-1-yl)methyl]-6-methoxy-3H-quinazolin-4-one;
Example #92. 6-(dimethylamino)-2-[(3,3-dimethylindolin-1-yl)methyl]-3H-pyrido[3,4-d]pyrimidin-4-one;
Example #77. 2-[(3,3-dimethylindolin-1-yl)methyl]-7-fluoro-6-methoxy-3H-quinazolin-4-one;
Example #93. 7-chloro-2-[(3,3-dimethylindolin-1-yl)methyl]-6-methoxy-3H-quinazolin-4-one;
Example #2. 2-[(3,3-dimethylindolin-1-yl)methyl]-6-methoxy-3H-quinazolin-4-one;
Example #4. 6-methoxy-2-[(3-methylindolin-1-yl)methyl]-3H-quinazolin-4-one, enantiomer 1;
Example #74. 6-(dimethylamino)-2-[(3,3-dimethylindolin-1-yl)methyl]-3H-quinazolin-4-one;
Example #6. 2-[(3-isopropylindolin-1-yl)methyl]-6-methoxy-3H-quinazolin-4-one;
Example #80. 6-(dimethylamino)-7-fluoro-2-(indolin-1-ylmethyl)-3H-quinazolin-4-one;
Example #100. 6-(azetidin-1-yl)-2-(indolin-1-ylmethyl)-3H-quinazolin-4-one;
Example #3. 6-methoxy-2-[(3-methylindolin-1-yl)methyl]-3H-quinazolin-4-one;
Example #1. 2-(indolin-1-ylmethyl)-6-methoxy-3H-quinazolin-4-one;
Example #73. 6-(dimethylamino)-2-(indolin-1-ylmethyl)-3H-quinazolin-4-one;
Example #81. 2-(indolin-1-ylmethyl)-6-isopropyl-3H-quinazolin-4-one;
Example #7. 2-[(6-fluoroindolin-1-yl)methyl]-6-methoxy-3H-quinazolin-4-one;
Example #22. 6-methoxy-2-(2,3,4,5-tetrahydro-1-benzazepin-1-ylmethyl)-3H-quinazolin-4-one;
Example #76. 7-fluoro-2-(indolin-1-ylmethyl)-6-methoxy-3H-quinazolin-4-one;
Example #38. 6-chloro-2-[(3-methylindolin-1-yl)methyl]-3H-quinazolin-4-one enantiomer 1;
Example #12. 2-[(8-chloro-3,4-dihydro-2H-quinolin-1-yl)methyl]-6-methoxy-3H-quinazolin-4-one;
Example #79.2-[(3,3-dimethylindolin-1-yl)methyl]-6-methoxy-3H-pyrido[3,4-d]pyrimidin-4-one;
Example #103. 2-(indolin-1-ylmethyl)-6-[3-(methoxymethyl)pyrrolidin-1-yl]-3H-quinazolin-4-one;
Example #62. 6-chloro-2-(2,3,4,5-tetrahydro-1-benzazepin-1-ylmethyl)-3H-quinazolin-4-one;
Example #40. 6-chloro-2-[(3,3-dimethylindolin-1-yl)methyl]-3H-quinazolin-4-one;
Example #102. 2-(indolin-1-ylmethyl)-6-pyrrolidin-1-yl-3H-quinazolin-4-one;
Example #23. 6-methoxy-2-[(1-methyl-3,4-dihydro-2H-1,5-benzodiazepin-5-yl)methyl]-3H-quinazolin-4-one;
Example #53. 6-chloro-2-[(5-methyl-3,4-dihydro-2H-quinolin-1-yl)methyl]-3H-quinazolin-4-one;
Example #84. 2-(indolin-1-ylmethyl)-6-methyl-3H-quinazolin-4-one;
Example #16. 2-[(6-methoxy-4-oxo-3H-quinazolin-2-yl)methyl]-3,4-dihydro-1H-isoquinoline-5-carbonitrile;
Example #58. 6-chloro-2-[(5-methoxy-3,4-dihydro-1H-isoquinolin-2-yl)methyl]-3H-quinazolin-4-one;
Example #59A. 6-chloro-2-(1,2,3,4-tetrahydroisoquinolin-2-ium-2-ylmethyl)-3H-quinazolin-4-one chloride;
Example #17. 6-methoxy-2-[(5-methoxy-3,4-dihydro-1H-isoquinolin-2-yl)methyl]-3H-quinazolin-4-one;
Example #60. 6-chloro-2-[(7-methyl-3,4-dihydro-1H-isoquinolin-2-yl)methyl]-3H-quinazolin-4-one; and
Example #39. 6-chloro-2-[(3-methylindolin-1-yl)methyl]-3H-quinazolin-4-one enantiomer 2.

Another object of the invention a compound of formula (I) as defined above for use as a drug. A further object of the invention is a pharmaceutical composition comprising a compound of formula (I) as defined above, and an acceptable pharmaceutical excipient. In another further particular embodiment, the present invention relates to a compound of formula (I), or a compound selected in the group consisting of 6-chloro-2-(indolin-1-ylmethyl)-3H-quinazolin-4-one; 6-chloro-2-(3,4-dihydro-2H-quinolin-1-ylmethyl)-3H-quinazolin-4-one; 6-chloro-2-(1,2,3,4-tetrahydroisoquinolin-2-ium-2-ylmethyl)-3H-quinazolin-4-one; 6-chloro-2-[(4-hydroxy-5,8-dimethoxy-3,4-dihydro-1H-isoquinolin-2-yl)methyl]-3H-quinazolin-4-one; 2-(indolin-1-ylmethyl)-6,7-dimethoxy-3H-quinazolin-4-one; and 7-chloro-2-(indolin-1-ylmethyl)-3H-quinazolin-4-one, or a pharmaceutical composition comprising the same for use for treating a cancer.

### DETAILED DESCRIPTION OF THE INVENTION

### Definitions

According to the Present Invention, the Terms Below have the Following Meanings:

The terms mentioned herein with prefixes such as for example C₁-C₆, can also be used with lower numbers of carbon atoms such as C₁-C₂. If, for example, the term C₁-C₆ is used, it means that the corresponding hydrocarbon chain may comprise from 1 to 6 carbon atoms, especially 1, 2, 3, 4, 5, or 6 carbon atoms. If, for example, the term C₁-C₃ is used, it means that the corresponding hydrocarbon chain may comprise from 1 to 3 carbon atoms, especially 1, 2, or 3 carbon atoms.

The term “alkyl” refers to a saturated, linear or branched aliphatic group. The term “(C₁-C₆)alkyl” more specifically means methyl, ethyl, propyl, isopropyl, butyl, pentyl, or hexyl. In a preferred embodiment, the “alkyl” is a methyl.

The term “alkoxy” or “alkyloxy” corresponds to the alkyl group as above defined bonded to the molecule by an —O— (ether) bond. (C₁-C₆)alkoxy includes methoxy, ethoxy, propyloxy, isopropyloxy, butyloxy, pentyloxy, or hexyloxy In a preferred embodiment, the “alkoxy” or “alkyloxy” is a methoxy, an ethoxy, a propoxy, an isopropyloxy, more preferably a methoxy.

The term “cycloalkyl” corresponds to a saturated or unsaturated mono-, bi- or tri-cyclic alkyl group comprising between 3 and 20 atoms of carbons. It also includes fused, bridged, or spiro-connected cycloalkyl groups. The term “cycloalkyl” includes for instance cyclopropyl, cyclobutyl, cyclopentyl, and cyclohexyl, preferably cyclopropyl. The term “spirocycloalkyl” includes for instance a spirocyclopentyl.

The term “heterocycloalkyl” corresponds to a saturated or unsaturated cycloalkyl group as above defined further comprising at least one heteroatom such as nitrogen, oxygen, or sulphur atom, preferably at least one nitrogen atom. It also includes fused, bridged, or spiro-connected heterocycloalkyl groups. Representative heterocycloalkyl groups include, but are not limited to dioxolanyl, benzo[1,3]dioxolyl, azetidinyl, oxetanyl, pyrazolinyl, pyranyl, thiomorpholinyl, pyrazolidinyl, piperidyl, piperazinyl, 1,4-dioxanyl, imidazolinyl, pyrrolinyl, pyrrolidinyl, piperidinyl, imidazolidinyl, morpholinyl, 1,4-dithianyl, pyrrolidinyl, oxozolinyl, oxazolidinyl, isoxazolinyl, isoxazolidinyl, thiazolinyl, thiazolidinyl, isothiazolinyl, isothiazolidinyl, dihydropyranyl, tetrahydropyranyl, tetrahydrofuranyl, and tetrahydrothiophenyl. In a preferred embodiment, the heterocycloalkyl group is azetidinyl, pyrrolidinyl, and piperidinyl.

The term “aryl” corresponds to a mono- or bi-cyclic aromatic hydrocarbons having from 6 to 12 carbon atoms. For instance, the term “aryl” includes phenyl, naphthyl, or anthracenyl. In a preferred embodiment, the aryl is a phenyl.

The term “heteroaryl” as used herein corresponds to an aromatic, mono- or poly-cyclic group comprising between 5 and 14 atoms and comprising at least one heteroatom such as nitrogen, oxygen or sulphur atom. As used herein, the term “heteroaryl” further includes the “fused arylheterocycloalkyl” and “fused heteroarylcycloalkyl”. The terms “fused arylheterocycloalkyl” and “fused heteroarylcycloalkyl” correspond to a bicyclic group in which an aryl as above defined or a heteroaryl is respectively bounded to the heterocycloalkyl or the cycloalkyl as above defined by at least two carbons. In other terms, the aryl or the heteroaryl shares a carbon bond with the heterocycloalkyl or the cycloalkyl. Examples of such mono- and poly-cyclic heteroaryl group, fused arylheterocycloalkyl and fused arylcycloalkyl may be: pyridinyl, thiazolyl, thiophenyl, furanyl, pyrrolyl, pyrazolyl, imidazolyl, triazolyl, tetrazolyl, benzofuranyl, thianaphthalenyl, indolyl, indolinyl, quinolinyl, isoquinolinyl, benzimidazolyl, tetrahydroquinolinyl, tetrahydroisoquinolinyl, triazinyl, thianthrenyl, isobenzofuranyl, chromenyl, xanthenyl, phenoxanthinyl, isothiazolyl, isoxazolyl, pyrazinyl, pyridazinyl, indolizinyl, isoindolyl, indazolyl, purinyl, quinolizinyl, phtalazinyl, naphthyridinyl, quinoxalinyl, quinazolinyl, cinnolinyl, pteridinyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, indolinyl, isoindolinyl, oxazolidinyl, benzotriazolyl, benzoisoxazolyl, oxindolyl, benzoxazolyl, benzoxazolinyl, benzoxazinyl, benzothienyl, benzothiazolyl, benzodiazepinyl, benzazepinyl, benzoxazepinyl, isatinyl, dihydropyridyl, pyrimidinyl, s-triazinyl, oxazolyl, or thiofuranyl. A fused arylheterocycloalkyl is for instance an indolinyl (phenyl fused to a pyrrolidinyl), a 1,2,3,4-tetrahydroquinolinyl or a 1,2,3,4-tetrahydroisoquinolinyl (phenyl fused to a piperidinyl), a 3,4-dihydro-1,4-benzoxazinyl (phenyl fused to a morpholinyl), a 2,3,4,5-tetrahydro-1,5-benzodiazepinyl (phenyl fused to a diazepinyl), a 2,3,4,5-tetrahydro-1-benzazepinyl (phenyl fused to an azepinyl), and a 2,3,4,5-tetrahydro-1,5-benzoxazepinyl (phenyl fused to an oxazepinyl). In a preferred embodiment, a fused arylheterocycloalkyl is indolinyl, 1,2,3,4-tetrahydroquinolinyl, 1,2,3,4-tetrahydroisoquinolinyl, and 2,3,4,5-tetrahydro-1,5-benzodiazepinyl.

The term “halogen” corresponds to a fluorine, chlorine, bromine, or iodine atom, preferably a fluorine or a chlorine.

The expression “substituted by at least” means that the radical is substituted by one or several groups of the list. For instance, the expression “a (C₁-C₆)alkyl substituted by at least one halogen may include a fluoromethyl (—CH₂F), a difluoromethyl (—CHF₂), or a trifluormethyl (—CF₃).

The expression “optionally substituted” means that the radical is not substituted or substituted by one or several groups of the list.

The “stereoisomers” are isomeric compounds that have the same molecular formula and sequence of bonded atoms, but differ in the 3D-dimensional orientations of their atoms in space. The stereoisomers include enantiomers, diastereoisomers, Cis-trans and E-Z isomers, conformers, and anomers. In a preferred embodiment of the invention, the stereoisomers include diastereoisomers and enantiomers. The enantiomers compounds may be prepared from the racemate compound using any purification method known by a skilled person, such as LC/MS and chiral HPLC analysis methods such as those disclosed in the examples (Example A—Chemistry, Table 1 and Table 2).

The “tautomers” are isomeric compounds that differ only in the position of the protons and the electrons. A tautomer of the compound of formula (I) can be represented by the following formula:

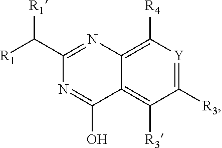

in which R₁, R_{1′}, Y, R₃, R_{Y}, and R₄ are such as defined herein.

COR refers to C(O)—R and CO₂R refers to C(O)—O—R.

The “pharmaceutically salts” include inorganic as well as organic acids salts. Representative examples of suitable inorganic acids include hydrochloric, hydrobromic, hydroiodic, phosphoric, and the like. Representative examples of suitable organic acids include formic, acetic, trichloroacetic, trifluoroacetic, propionic, benzoic, cinnamic, citric, fumaric, maleic, methanesulfonic and the like. Further examples of pharmaceutically inorganic or organic acid addition salts include the pharmaceutically salts listed in J. Pharm. Sci. 1977, 66, 2, and in Handbook of Pharmaceutical Salts: Properties, Selection, and Use edited by P. Heinrich Stahl and Camille G. Wermuth 2002. In a preferred embodiment, the salt is selected from the group consisting of maleate, chlorhydrate, bromhydrate, and methanesulfonate. The “pharmaceutically salts” also include inorganic as well as organic base salts. Representative examples of suitable inorganic bases include sodium or potassium salt, an alkaline earth metal salt, such as a calcium or magnesium salt, or an ammonium salt. Representative examples of suitable salts with an organic base includes for instance a salt with methylamine, dimethylamine, trimethylamine, piperidine, morpholine or tris-(2-hydroxyethyl)amine. In a preferred embodiment, the salt is selected from the group consisting of sodium and potassium salt.

As used herein, the terms “treatment”, “treat” or “treating” refer to any act intended to ameliorate the health status of patients such as therapy, prevention, prophylaxis and retardation of a disease, in particular a cancer. In certain embodiments, such terms refer to the amelioration or eradication of the disease, or symptoms associated with it. In other embodiments, this term refers to minimizing the spread or worsening of the disease, resulting from the administration of one or more therapeutic agents to a subject with such a disease.

As used herein, the terms “subject”, “individual” or “patient” are interchangeable and refer to an animal, preferably to a mammal, even more preferably to a human, including adult, child, newborn and human at the prenatal stage. However, the term “subject” can also refer to non-human animals, in particular mammals such as dogs, cats, horses, cows, pigs, sheep and non-human primates, among others.

The terms “quantity,” “amount,” and “dose” are used interchangeably herein and may refer to an absolute quantification of a molecule.

As used herein, the terms “active principle”, “active ingredient” and “active pharmaceutical ingredient” are equivalent and refers to a component of a pharmaceutical composition having a therapeutic effect.

As used herein, the term “therapeutic effect” refers to an effect induced by an active ingredient, or a pharmaceutical composition according to the invention, capable to prevent or to delay the appearance or development of a disease or disorder, or to cure or to attenuate the effects of a disease or disorder.

As used herein, the term “effective amount” refers to a quantity of an active ingredient or of a pharmaceutical composition which prevents, removes or reduces the deleterious effects of the disease, particularly a cancer. It is obvious that the quantity to be administered can be adapted by the man skilled in the art according to the subject to be treated, to the nature of the disease, etc. In particular, doses and regimen of administration may be function of the nature, of the stage and of the severity of the disease to be treated, as well as of the weight, the age and the global health of the subject to be treated, as well as of the judgment of the doctor.

As used herein, the term “pharmaceutically acceptable excipient” refers to any ingredient except active ingredients which are present in a pharmaceutical composition. Its addition may be aimed to confer a particular consistency or other physical or gustative properties to the final product.

A pharmaceutically acceptable excipient must be devoid of any interaction, in particular chemical, with the active ingredients.

### Compounds

The present invention provides new compounds of therapeutic interest.

According to the invention, a compound has the following formula (I):

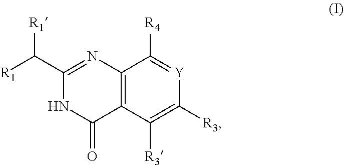

wherein:
R₁ represents a fused arylheterocycloalkyl selected in the group consisting of:
   an indolinyl,
   an isoindolinyl,
   a 1,2,3,4-tetrahydroquinolinyl,
   a 1,2,3,4-tetrahydroisoquinolinyl,
   a 3,4-dihydro-1,4-benzoxazinyl,
   a 2,3-dihydroquinoxalinyl,
   a 2,3,4,5-tetrahydro-1,5-benzodiazepinyl
   a 2,3,4,5-tetrahydro-1-benzazepinyl, and
   a 2,3,4,5-tetrahydro-1,5-benzoxazepinyl,
said fused arylheterocycloalkyl is optionally substituted by at least one radical selected in the group consisting of:
   a halogen,
   a (C₁-C₆)alkyl optionally substituted by at least one hydroxy, an amino or a halogen,
   a (C₁-C₆)alkyloxy optionally substituted by at least one hydroxy, an amino or a halogen,
   a cyano,
   a cycloalkyl or a spirocycloalkyl,
   a hydroxy,
   a —NH—SO₂(CH₃),
   a —NR₅R₆ with R₅ and R₆ being independently a hydrogen, a (C₁-C₆)alkyl or a —COR₈ with R₈ being a (C₁-C₆)alkyl, and
R_{1′} represents a hydrogen or a (C₁-C₆)alkyl;
Y represents:
   a nitrogen, or
   a —CR₂ group in which R₂ is a radical selected in the group consisting of a hydrogen, a halogen, a (C₁-C₆)alkyl optionally substituted by at least one halogen, and a (C₁-C₆)alkyloxy optionally substituted by at least one halogen;
R₃ and R_{3′} represents independently a radical selected in the group consisting of:
   a 3-10 membered ring selected in the group consisting of a cycloalkyl, a heterocycloalkyl, and an aryl, said 3-10 membered ring is optionally substituted by a radical selected in a group consisting of:
      a (C₁-C₆)alkyl optionally substituted by a (C₁-C₆)alkyloxy, a hydroxy, a halogen, or a —NR₅R₆ with R₅ and R₆ being independently a hydrogen,
      a (C₁-C₆)alkyloxy optionally substituted by a a halogen,
      a halogen, and
      a hydroxy,
   a (C₁-C₆)alkyl and a (C₁-C₆)alkyloxy optionally substituted by a halogen, a (C₁-C₆)alkyloxy or a —NH—CO₂—R₇ with R₇ being a (C₁-C₆)alkyl,
   a —NR₅R₆ with R₅ and R₆ being independently a hydrogen or a (C₁-C₆)alkyl,
   a halogen,
   a hydrogen, and
   a cyano or
R₃ may form with R₂ a dioxolanyl; and
R₄ represents a hydrogen or a (C₁-C₆)alkyloxy;
   and the stereoisomers, the tautomers, and the pharmaceutical salts thereof.

More particularly, a compound of the invention has the following formula (I):

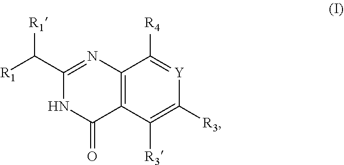

wherein:
R₁ represents a fused arylheterocycloalkyl selected in the group consisting of:
   an indolinyl,
   an isoindolinyl,
   a 1,2,3,4-tetrahydroquinolinyl,
   a 1,2,3,4-tetrahydroisoquinolinyl,
   a 3,4-dihydro-1,4-benzoxazinyl,
   a 2,3-dihydroquinoxalinyl,
   a 2,3,4,5-tetrahydro-1,5-benzodiazepinyl
   a 2,3,4,5-tetrahydro-1-benzazepinyl, and
   a 2,3,4,5-tetrahydro-1,5-benzoxazepinyl,
said fused arylheterocycloalkyl is optionally substituted by at least one radical selected in the group consisting of:
   a halogen,
   a (C₁-C₆)alkyl optionally substituted by at least one hydroxy, an amino, or a halogen,
   a (C₁-C₆)alkyloxy optionally substituted by at least one hydroxy, an amino, or a halogen,
   a cyano,
   a cycloalkyl or a spirocycloalkyl,
   a hydroxy,
   a —NH—SO₂(CH₃), and
   a —NR₅R₆ with R₅ and R₆ being independently a hydrogen or a (C₁-C₆)alkyl,
R_{1′} represents a hydrogen or a (C₁-C₆)alkyl;
Y represents:
   a nitrogen, or
   a —CR₂ group in which R₂ is a radical selected in the group consisting of a hydrogen, a halogen, a (C₁-C₆)alkyl optionally substituted by at least one halogen, a (C₁-C₆)alkyloxy optionally substituted by at least one halogen;
R₃ represents a radical selected in the group consisting of:
   a 3-10 membered ring selected in the group consisting of a cycloalkyl, a heterocycloalkyl, and an aryl, said 3-10 membered ring is optionally substituted by at least one radical selected in a group consisting of:
      a (C₁-C₆)alkyl optionally substituted by a (C₁-C₆)alkyloxy, a hydroxy or a halogen,
      a (C₁-C₆)alkyloxy optionally substituted by a halogen,
      a halogen, and
      a hydroxy
   a (C₁-C₆)alkyl and a (C₁-C₆)alkyloxy optionally substituted by a halogen, a (C₁-C₆)alkyloxy, or a —NH—CO₂—R₇ with R₇ being a (C₁-C₆)alkyl,
   a —NR₅R₆ with R₅ and R₆ being independently a hydrogen or a (C₁-C₆)alkyl,
   a halogen,
   a hydrogen, and
   a cyano, or
R₃ may form with R₂ a dioxolanyl;
R_{3′} represents a hydrogen; and
R₄ represents a hydrogen or a (C₁-C₆)alkyloxy;
   and the stereoisomers, the tautomers, and the pharmaceutical salts thereof.

According to the invention, a compound of formula (I) is not:
6-chloro-2-(indolin-1-ylmethyl)-3H-quinazolin-4-one;
6-chloro-2-(3,4-dihydro-2H-quinolin-1-ylmethyl)-3H-quinazolin-4-one;
6-chloro-2-(3,4-dihydro-1H-isoquinolin-2-ylmethyl)-3H-quinazolin-4-one;
6-chloro-2-[(4-hydroxy-5,8-dimethoxy-3,4-dihydro-1H-isoquinolin-2-yl)methyl]-3H-quinazolin-4-one;
2-(indolin-1-ylmethyl)-6,7-dimethoxy-3H-quinazolin-4-one; and
7-chloro-2-(indolin-1-ylmethyl)-3H-quinazolin-4-one.

In a particular embodiment, Y represents a —CR₂ group with R₂ is a radical selected in the group consisting of a hydrogen, a halogen, a (C₁-C₆)alkyl optionally substituted by at least one halogen, a (C₁-C₆)alkyloxy optionally substituted by at least one halogen. In a preferred embodiment, R₂ is a radical selected in the group consisting of a hydrogen, a halogen, preferably a fluorine or a chlorine, a (C₁-C₆)alkyloxy optionally substituted by a halogen, preferably a methoxy or a trifluoromethoxy, a (C₁-C₆)alkyl optionally substituted by a halogen, preferably a methyl or a trifluoromethyl, more preferably R₂ is a hydrogen or a halogen, preferably a fluorine and a chlorine, and even more preferably R₂ is a hydrogen. In a further preferred embodiment, R₂ is a hydrogen, a fluorine, a chlorine, a methoxy, or a trifluoromethoxy. In an even more preferred embodiment, R₂ is a hydrogen, a fluorine, or a chlorine.

In a further particular embodiment, Y represents a nitrogen.

In a particular embodiment, R₃, represents a hydrogen.

In a particular embodiment, R₃ represents a radical selected in the group consisting of:
a 3-10 membered ring selected in the group consisting of a cycloalkyl, a heterocycloalkyl, and an aryl, said 3-10 membered ring is optionally substituted by at least one radical selected in a group consisting of:
   a (C₁-C₆)alkyl optionally substituted by a (C₁-C₆)alkyloxy, a hydroxy or a halogen,
   a (C₁-C₆)alkyloxy optionally substituted by a halogen,
   a halogen, and
   a hydroxy
a (C₁-C₆)alkyl and a (C₁-C₆)alkyloxy optionally substituted by a halogen, a (C₁-C₆)alkyloxy, or a —NH—CO₂—R₇ with R₇ being a (C₁-C₆)alkyl,
a —NR₅R₆ with R₅ and R₆ being independently a hydrogen or a (C₁-C₆)alkyl,
a halogen,
a hydrogen, and
a cyano.

In a preferred embodiment, R₃ represents a radical selected in the group consisting of:
a 3-10 membered ring selected in the group consisting of a cyclopropyl, an azetidinyl, a pyrrolidinyl, a phenyl, and a piperidinyl, said 3-10 membered ring is optionally substituted by a radical selected in a group consisting of:
   a methyl substituted by a methoxy or a hydroxy,
   a (C₁-C₆)alkyloxy, preferably a methoxy, and
   a hydroxy,
a (C₁-C₆)alkyloxy, preferably a methoxy, a trifluromethoxy, an isopropyloxy, an ethoxy, a propoxy, or a (C₁-C₆)alkyloxy substituted by a methoxy or a —O—(CH₂)₄NHCO₂tBu,
a methyl, a trifluoromethyl, or an isopropyl,
a —NR₅R₆ with R₅ and R₆ being a methyl,
a chlorine or a flurorine, preferably a chlorine,
a hydrogen, and
a cyano.

In a further particular embodiment, R₃ represents a radical selected in the group consisting of a cycloalkyl, a (C₁-C₆)alkyloxy, a —NR₅R₆ with R₅ and R₆ being independently a hydrogen or a (C₁-C₆)alkyl, a heterocycloalkyl optionally substituted by a (C₁-C₆)alkyl, said (C₁-C₆)alkyl is optionally substituted by a (C₁-C₆)alkyloxy, a (C₁-C₆)alkyl, a halogen, and a (C₁-C₆)alkyl and a (C₁-C₆)alkyloxy optionally substituted by a halogen.

In a further preferred embodiment, R₃ represents a radical selected in the group consisting of a cyclopropyl, a methoxy, a —NR₅R₆ with R₅ and R₆ being a methyl, an azetidinyl, an isopropyl, a chlorine, a pyrrolidine optionally substituted by a —CH₂—O—CH₃, and a methyl.

In a particular embodiment, R₃ may form with R₂ a dioxolanyl.

In a particular embodiment, R₄ represents a hydrogen or a (C₁-C₆)alkyloxy. In a preferred embodiment, R₄ represents a hydrogen or a methoxy. In an even more preferred embodiment, R₄ represents a hydrogen.

In a particular embodiment, Ry represents a hydrogen or a (C₁-C₆)alkyl. In a preferred embodiment, R₄ represents a hydrogen or a methyl. In an even more preferred embodiment, R₄, represents a hydrogen.

In a preferred embodiment, a compound of formula (I) is selected in the group consisting of:
Example #99. 6-cyclopropyl-2-(indolin-1-ylmethyl)-3H-quinazolin-4-one;
Example #11. 2-[(8-fluoro-3,4-dihydro-2H-quinolin-1-yl)methyl]-6-methoxy-3H-quinazolin-4-one;
Example #92. 6-(dimethylamino)-2-[(3,3-dimethylindolin-1-yl)methyl]-3H-pyrido[3,4-d]pyrimidin-4-one;
Example #77. 2-[(3,3-dimethylindolin-1-yl)methyl]-7-fluoro-6-methoxy-3H-quinazolin-4-one;
Example #93. 7-chloro-2-[(3,3-dimethylindolin-1-yl)methyl]-6-methoxy-3H-quinazolin-4-one;
Example #2. 2-[(3,3-dimethylindolin-1-yl)methyl]-6-methoxy-3H-quinazolin-4-one;
Example #4. 6-methoxy-2-[(3-methylindolin-1-yl)methyl]-3H-quinazolin-4-one, enantiomer 1;
Example #74. 6-(dimethylamino)-2-[(3,3-dimethylindolin-1-yl)methyl]-3H-quinazolin-4-one;
Example #6. 2-[(3-isopropylindolin-1-yl)methyl]-6-methoxy-3H-quinazolin-4-one;
Example #80. 6-(dimethylamino)-7-fluoro-2-(indolin-1-ylmethyl)-3H-quinazolin-4-one;
Example #100. 6-(azetidin-1-yl)-2-(indolin-1-ylmethyl)-3H-quinazolin-4-one;
Example #3. 6-methoxy-2-[(3-methylindolin-1-yl)methyl]-3H-quinazolin-4-one;
Example #1. 2-(indolin-1-ylmethyl)-6-methoxy-3H-quinazolin-4-one;
Example #73. 6-(dimethylamino)-2-(indolin-1-ylmethyl)-3H-quinazolin-4-one;
Example #81. 2-(indolin-1-ylmethyl)-6-isopropyl-3H-quinazolin-4-one;
Example #7. 2-[(6-fluoroindolin-1-yl)methyl]-6-methoxy-3H-quinazolin-4-one;
Example #22. 6-methoxy-2-(2,3,4,5-tetrahydro-1-benzazepin-1-ylmethyl)-3H-quinazolin-4-one;
Example #76. 7-fluoro-2-(indolin-1-ylmethyl)-6-methoxy-3H-quinazolin-4-one;
Example #38. 6-chloro-2-[(3-methylindolin-1-yl)methyl]-3H-quinazolin-4-one enantiomer 1;
Example #12. 2-[(8-chloro-3,4-dihydro-2H-quinolin-1-yl)methyl]-6-methoxy-3H-quinazolin-4-one;
Example #13. 6-methoxy-2-[(8-methoxy-3,4-dihydro-2H-quinolin-1-yl)methyl]-3H-quinazolin-4-one;
Example #79. 2-[(3,3-dimethylindolin-1-yl)methyl]-6-methoxy-3H-pyrido[3,4-d]pyrimidin-4-one;
Example #103. 2-(indolin-1-ylmethyl)-6-[3-(methoxymethyl)pyrrolidin-1-yl]-3H-quinazolin-4-one;
Example #62. 6-chloro-2-(2,3,4,5-tetrahydro-1-benzazepin-1-ylmethyl)-3H-quinazolin-4-one;
Example #43. 6-chloro-2-[(4-methylindolin-1-yl)methyl]-3H-quinazolin-4-one;
Example #82. 2-(indolin-1-ylmethyl)-6-(trifluoromethoxy)-3H-quinazolin-4-one;
Example #40. 6-chloro-2-[(3,3-dimethylindolin-1-yl)methyl]-3H-quinazolin-4-one;
Example #44. 6-chloro-2-[(7-methylindolin-1-yl)methyl]-3H-quinazolin-4-one;
Example #52. 6-chloro-2-[(5-methoxy-3,4-dihydro-2H-quinolin-1-yl)methyl]-3H-quinazolin-4-one;
Example #102. 2-(indolin-1-ylmethyl)-6-pyrrolidin-1-yl-3H-quinazolin-4-one;
Example #23. 6-methoxy-2-[(1-methyl-3,4-dihydro-2H-1,5-benzodiazepin-5-yl)methyl]-3H-quinazolin-4-one;
Example #14. 6-methoxy-2-[(5-methoxy-2,3-dihydro-1,4-benzoxazin-4-yl)methyl]-3H-quinazolin-4-one;
Example #53. 6-chloro-2-[(5-methyl-3,4-dihydro-2H-quinolin-1-yl)methyl]-3H-quinazolin-4-one;
Example #84. 2-(indolin-1-ylmethyl)-6-methyl-3H-quinazolin-4-one;
Example #16. 2-[(6-methoxy-4-oxo-3H-quinazolin-2-yl)methyl]-3,4-dihydro-1H-isoquinoline-5-carbonitrile;
Example #54. 6-chloro-2-[(8-methoxy-3,4-dihydro-2H-quinolin-1-yl)methyl]-3H-quinazolin-4-one;
Example #37. 6-chloro-2-[(3-methylindolin-1-yl)methyl]-3H-quinazolin-4-one or its tautomer 6-chloro-2-[(3-methylindolin-1-yl)methyl]-quinazolin-4-ol;
Example #78. 2-(indolin-1-ylmethyl)-6-methoxy-3H-pyrido[3,4-d]pyrimidin-4-one;
Example #15. 2-(3,4-dihydro-2H-quinolin-1-ylmethyl)-6-methoxy-3H-quinazolin-4-one;
Example #5. 6-methoxy-2-[(3-methylindolin-1-yl)methyl]-3H-quinazolin-4-one, enantiomer 2;
Example #104. 6-[3-(hydroxymethyl)pyrrolidin-1-yl]-2-(indolin-1-ylmethyl)-3H-quinazolin-4-one;
Example #42. 6-chloro-2-(spiro[cyclopentane-1,3′-indoline]-1′-ylmethyl)-3H-quinazolin-4-one;
Example #85. 2-(indolin-1-ylmethyl)-6-(trifluoromethyl)-3H-quinazolin-4-one;
Example #86. 2-(indolin-1-ylmethyl)-7-methoxy-3H-quinazolin-4-one;
Example #87. 2-(indolin-1-ylmethyl)-6-isopropoxy-3H-quinazolin-4-one;
Example #47. 6-chloro-2-[(5-fluoroindolin-1-yl)methyl]-3H-quinazolin-4-one (;
Example #63. 6-chloro-2-[(1-methyl-3,4-dihydro-2H-1,5-benzodiazepin-5-yl)methyl]-3H-quinazolin-4-one;
Example #45. 6-chloro-2-[(6-methylindolin-1-yl)methyl]-3H-quinazolin-4-one;
Example #88. 6-fluoro-2-(indolin-1-ylmethyl)-3H-quinazolin-4-one;
Example #58. 6-chloro-2-[(5-methoxy-3,4-dihydro-1H-isoquinolin-2-yl)methyl]-3H-quinazolin-4-one;
Example #59A. 6-chloro-2-(1,2,3,4-tetrahydroisoquinolin-2-ium-2-ylmethyl)-3H-quinazolin-4-one chloride;
Example #64. 6-chloro-2-(3,4-dihydro-2H-1,5-benzoxazepin-5-ylmethyl)-3H-quinazolin-4-one;
Example #46. 6-chloro-2-[(5-methylindolin-1-yl)methyl]-3H-quinazolin-4-one;
Example #94. 2-[(3,3-dimethylindolin-1-yl)methyl]-6,8-dimethoxy-3H-quinazolin-4-one;
Example #48. 2-[(5-bromoindolin-1-yl)methyl]-6-chloro-3H-quinazolin-4-one;
Example #17. 6-methoxy-2-[(5-methoxy-3,4-dihydro-1H-isoquinolin-2-yl)methyl]-3H-quinazolin-4-one;
Example #10. 2-(isoindolin-2-ylmethyl)-6-methoxy-3H-quinazolin-4-one;
Example #119. 6-[(3,3-dimethylindolin-1-yl)methyl]-7H-[1,3]dioxolo[4,5-g]quinazolin-8-one;
Example #97. 2-[(3,3-dimethylindolin-1-yl)methyl]-6-(2-methoxyethoxy)-3H-quinazolin-4-one;
Example #120. 6-chloro-2-(1-indolin-1-ylethyl)-3H-quinazolin-4-one;
Example #8. 2-[(7-fluoroindolin-1-yl)methyl]-6-methoxy-3H-quinazolin-4-one;
Example #90. tert-butyl N-[4-[[2-(indolin-1-ylmethyl)-4-oxo-3H-quinazolin-6-yl]oxy]butyl]carbamate;
Example #60. 6-chloro-2-[(7-methyl-3,4-dihydro-1H-isoquinolin-2-yl)methyl]-3H-quinazolin-4-one;
Example #18. 2-[(5-bromo-3,4-dihydro-1H-isoquinolin-2-yl)methyl]-6-methoxy-3H-quinazolin-4-one;
Example #41. 6-chloro-2-[(2-methylindolin-1-yl)methyl]-3H-quinazolin-4-one;
Example #91. 2-(indolin-1-ylmethyl)-7-(trifluoromethyl)-3H-quinazolin-4-one;
Example #19. 6-methoxy-2-[(8-methoxy-3,4-dihydro-1H-isoquinolin-2-yl)methyl]-3H-quinazolin-4-one;
Example #101. 2-(indolin-1-ylmethyl)-6-(3-methoxyazetidin-1-yl)-3H-quinazolin-4-one;
Example #39. 6-chloro-2-[(3-methylindolin-1-yl)methyl]-3H-quinazolin-4-one enantiomer 2;
Example #96. 2-[(3,3-dimethylindolin-1-yl)methyl]-6-(3-methoxypropoxy)-3H-quinazolin-4-one;
Example #20. 2-[(5-chloro-3,4-dihydro-1H-isoquinolin-2-yl)methyl]-6-methoxy-3H-quinazolin-4-one;
Example #55. 6-chloro-2-[(7-methoxy-3,4-dihydro-2H-quinolin-1-yl)methyl]-3H-quinazolin-4-one;
Example #106. 2-(indolin-1-ylmethyl)-6-(1-piperidyl)-3H-quinazolin-4-one;
Example #21. N-[2-[(6-methoxy-4-oxo-3H-quinazolin-2-yl)methyl]-3,4-dihydro-1H-isoquinolin-5-yl]methanesulfonamide;
Example #49. 6-chloro-2-[(7-methoxyindolin-1-yl)methyl]-3H-quinazolin-4-one;
Example #95. 2-[(3,3-dimethylindolin-1-yl)methyl]-4-oxo-3H-quinazoline-6-carbonitrile;
Example #50. 6-chloro-2-(isoindolin-2-ylmethyl)-3H-quinazolin-4-one;
Example #105. 6-(3-hydroxypyrrolidin-1-yl)-2-(indolin-1-ylmethyl)-3H-quinazolin-4-one;
Example #122. 2-(3,4-dihydro-1H-isoquinolin-2-ylmethyl)-6-methoxy-3H-quinazolin-4-one;
Example #123. 2-[(4-hydroxy-3,4-dihydro-1H-isoquinolin-2-yl)methyl]-6-methoxy-3H-quinazolin-4-one;
Example #128. 6-chloro-2-[(4-methyl-2,3-dihydroquinoxalin-1-yl)methyl]-3H-quinazolin-4-one;
Example #129. 2-[[3-(2-hydroxyethyl)indolin-1-yl]methyl]-6-methoxy-3H-quinazolin-4-one;
Example #127. 6-chloro-2-[(7-methyl-3,4-dihydro-2H-quinolin-1-yl)methyl]-3H-quinazolin-4-one;
Example #124. 2-[(5-fluoro-3,4-dihydro-1H-isoquinolin-2-yl)methyl]-6-methoxy-3H-quinazolin-4-one;
Example #133. 2-[(5-amino-3,4-dihydro-1H-isoquinolin-2-yl)methyl]-6-methoxy-3H-quinazolin-4-one;
Example #132. 2-(indolin-1-ylmethyl)-6-(4-methoxy-1-piperidyl)-3H-quinazolin-4-one;
Example #131. 2-[1-[(6-methoxy-4-oxo-3H-quinazolin-2-yl)methyl]indolin-3-yl]ethylammonium chloride;
Example #138. 7-fluoro-2-(indolin-1-ylmethyl)-3H-quinazolin-4-one;
Example #144. 6-chloro-2-((5-methoxyindolin-1-yl)methyl)quinazolin-4(3H)-one;
Example #130. 6-chloro-2-[(8-methoxy-3,4-dihydro-1H-isoquinolin-2-yl)methyl]-3H-quinazolin-4-one;
Example #125. 6-methoxy-2-[[5-(trifluoromethyl)-3,4-dihydro-1H-isoquinolin-2-yl]methyl]-3H-quinazolin-4-one;
Example #126. 2-[(4-fluoroindolin-1-yl)methyl]-6-methoxy-3H-quinazolin-4-one;
Example #9. 2-[[3-(hydroxymethyl)indolin-1-yl]methyl]-6-methoxy-3H-quinazolin-4-one; and
Example #57. 6-chloro-2-(2,3-dihydro-1,4-benzoxazin-4-ylmethyl)-3H-quinazolin-4-one.

In a more preferred embodiment, a compound of formula (I) is selected in the group consisting of:
Example #99. 6-cyclopropyl-2-(indolin-1-ylmethyl)-3H-quinazolin-4-one;
Example #11. 2-[(8-fluoro-3,4-dihydro-2H-quinolin-1-yl)methyl]-6-methoxy-3H-quinazolin-4-one;
Example #92. 6-(dimethylamino)-2-[(3,3-dimethylindolin-1-yl)methyl]-3H-pyrido[3,4-d]pyrimidin-4-one;
Example #77. 2-[(3,3-dimethylindolin-1-yl)methyl]-7-fluoro-6-methoxy-3H-quinazolin-4-one;
Example #93. 7-chloro-2-[(3,3-dimethylindolin-1-yl)methyl]-6-methoxy-3H-quinazolin-4-one;
Example #2. 2-[(3,3-dimethylindolin-1-yl)methyl]-6-methoxy-3H-quinazolin-4-one;
Example #4. 6-methoxy-2-[(3-methylindolin-1-yl)methyl]-3H-quinazolin-4-one, enantiomer 1;
Example #74. 6-(dimethylamino)-2-[(3,3-dimethylindolin-1-yl)methyl]-3H-quinazolin-4-one;
Example #6. 2-[(3-isopropylindolin-1-yl)methyl]-6-methoxy-3H-quinazolin-4-one;
Example #80. 6-(dimethylamino)-7-fluoro-2-(indolin-1-ylmethyl)-3H-quinazolin-4-one;
Example #100. 6-(azetidin-1-yl)-2-(indolin-1-ylmethyl)-3H-quinazolin-4-one;
Example #3. 6-methoxy-2-[(3-methylindolin-1-yl)methyl]-3H-quinazolin-4-one;
Example #1. 2-(indolin-1-ylmethyl)-6-methoxy-3H-quinazolin-4-one;
Example #73. 6-(dimethylamino)-2-(indolin-1-ylmethyl)-3H-quinazolin-4-one;
Example #81. 2-(indolin-1-ylmethyl)-6-isopropyl-3H-quinazolin-4-one;
Example #7. 2-[(6-fluoroindolin-1-yl)methyl]-6-methoxy-3H-quinazolin-4-one;
Example #22. 6-methoxy-2-(2,3,4,5-tetrahydro-1-benzazepin-1-ylmethyl)-3H-quinazolin-4-one;
Example #76. 7-fluoro-2-(indolin-1-ylmethyl)-6-methoxy-3H-quinazolin-4-one;
Example #38. 6-chloro-2-[(3-methylindolin-1-yl)methyl]-3H-quinazolin-4-one enantiomer 1;
Example #12. 2-[(8-chloro-3,4-dihydro-2H-quinolin-1-yl)methyl]-6-methoxy-3H-quinazolin-4-one;
Example #79. 2-[(3,3-dimethylindolin-1-yl)methyl]-6-methoxy-3H-pyrido[3,4-d]pyrimidin-4-one;
Example #103. 2-(indolin-1-ylmethyl)-6-[3-(methoxymethyl)pyrrolidin-1-yl]-3H-quinazolin-4-one;
Example #62. 6-chloro-2-(2,3,4,5-tetrahydro-1-benzazepin-1-ylmethyl)-3H-quinazolin-4-one;
Example #40. 6-chloro-2-[(3,3-dimethylindolin-1-yl)methyl]-3H-quinazolin-4-one;
Example #102. 2-(indolin-1-ylmethyl)-6-pyrrolidin-1-yl-3H-quinazolin-4-one;
Example #23. 6-methoxy-2-[(1-methyl-3,4-dihydro-2H-1,5-benzodiazepin-5-yl)methyl]-3H-quinazolin-4-one;
Example #53. 6-chloro-2-[(5-methyl-3,4-dihydro-2H-quinolin-1-yl)methyl]-3H-quinazolin-4-one;
Example #84. 2-(indolin-1-ylmethyl)-6-methyl-3H-quinazolin-4-one;
Example #16. 2-[(6-methoxy-4-oxo-3H-quinazolin-2-yl)methyl]-3,4-dihydro-1H-isoquinoline-5-carbonitrile;
Example #58. 6-chloro-2-[(5-methoxy-3,4-dihydro-1H-isoquinolin-2-yl)methyl]-3H-quinazolin-4-one;
Example #59A. 6-chloro-2-(1,2,3,4-tetrahydroisoquinolin-2-ium-2-ylmethyl)-3H-quinazolin-4-one; chloride
Example #17. 6-methoxy-2-[(5-methoxy-3,4-dihydro-1H-isoquinolin-2-yl)methyl]-3H-quinazolin-4-one;
Example #60. 6-chloro-2-[(7-methyl-3,4-dihydro-1H-isoquinolin-2-yl)methyl]-3H-quinazolin-4-one; and
Example #39. 6-chloro-2-[(3-methylindolin-1-yl)methyl]-3H-quinazolin-4-one enantiomer 2.

According to the invention, R₁ represents a fused arylheterocycloalkyl selected in the group consisting of:
an indolinyl,
an isoindolinyl,
a 1,2,3,4-tetrahydroquinolinyl,
a 1,2,3,4-tetrahydroisoquinolinyl,
a 2,3-dihydroquinoxalinyl,
a 3,4-dihydro-1,4-benzoxazinyl,
a 2,3,4,5-tetrahydro-1,5-benzodiazepinyl
a 2,3,4,5-tetrahydro-1-benzazepinyl, and
a 2,3,4,5-tetrahydro-1,5-benzoxazepinyl,

said fused arylheterocycloalkyl is optionally substituted by at least one radical selected in the group consisting of:
a halogen,
a (C₁-C₆)alkyl optionally substituted by at least one hydroxy, an amino, or a halogen,
a (C₁-C₆)alkyloxy optionally substituted by at least one hydroxy, an amino, or a halogen,
a cyano,
a cycloalkyl or a spirocycloalkyl,
a hydroxy,
a —NH—SO₂(CH₃), and
a —NR₅R₆ with R₅ and R₆ being independently a hydrogen, a (C₁-C₆)alkyl, or a —COR₈ with R₈ being a (C₁-C₆)alkyl.

Preferably, R₁ represents a fused arylheterocycloalkyl selected in the group consisting of an indolinyl, a 1,2,3,4-tetrahydroquinolinyl, a 1,2,3,4-tetrahydroisoquinolinyl, a 2,3,4,5-tetrahydro-1,5-benzodiazepinyl, and a 2,3,4,5-tetrahydro-1-benzazepinyl, said fused arylheterocycloalkyl is optionally substituted by at least one radical as above defined.

An indolinyl has the following formula:

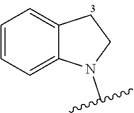 [SMILES: *N1CCc2ccccc21]

A 1,2,3,4-tetrahydroquinolinyl has the following formula:

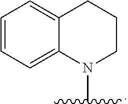 [SMILES: *N1CCCc2ccccc21]

A 1,2,3,4-tetrahydroisoquinolinyl has the following formula:

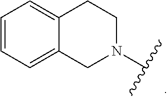 [SMILES: *N1CCc2ccccc2C1]

A 2,3,4,5-tetrahydro-1,5-benzodiazepinyl has the following formula:

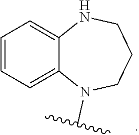 [SMILES: *N1CCCNc2ccccc21]

A 2,3,4,5-tetrahydro-1-benzazepinyl has the following formula:

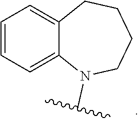 [SMILES: *N1CCCCc2ccccc21]

### Indolinyl Series

In an embodiment, R₁ represents an optionally substituted indolinyl or an optionally substituted isoindolinyl. In a further particular embodiment, R₁ represents an unsubstituted indolinyl or an indolinyl substituted by at least one radical as above defined, preferably by one or two radicals as above defined.

In a preferred embodiment, R₁ represents an unsubstituted indolinyl or an indolinyl substituted by at least one radical selected in the group consisting of:
a halogen,
a (C₁-C₆)alkyl optionally substituted by at least one hydroxy or an amino,
a (C₁-C₆)alkyloxy, and
a spirocycloalkyl, preferably a spirocyclopentyl.

In a more preferred embodiment, R₁ represents an unsubstituted indolinyl, an indolinyl substituted by at least one radical selected in the group consisting of a halogen, preferably a fluorine, and a (C₁-C₆)alkyl, preferably a methyl or an isopropyl, or an indolinyl substituted by two (C₁-C₆)alkyls, preferably two methyls.

As above defined, an indolinyl corresponds to a phenyl fused to a pyrrolidinyl. The indolinyl can be substituted on the phenyl and/or the pyrrolidinyl by at least one radical as defined above. In a particular embodiment, the indolinyl can be substituted on the phenyl by at least one radical selected from the group consisting of a (C₁-C₆)alkyl optionally substituted by at least one hydroxy, a halogen or an amino, a (C₁-C₆)alkoxy, and a halogen, preferably selected from the group consisting of methyl, methoxy, halogen and —CH₂—NH₂. In a further particular embodiment, the indolinyl can be substituted on the pyrrolidinyl, especially in position 3, by at least one radical selected from the group consisting of a (C₁-C₆)alkyl optionally substituted by at least one hydroxy or an amino, preferably selected from the group consisting of methyl, an isopropyl, and a spirocyclopentyl. In a particular aspect, the indolinyl is substituted on the pyrrolidinyl, especially in position 3, by one or two methyls, preferably by two methyls.

A preferred compound of formula (I) is such that:
R₁ represents an unsubstituted indolinyl or an indolinyl substituted by at least one radical selected in the group consisting of:
   a halogen, preferably substituted on the phenyl,
   a (C₁-C₆)alkyl optionally substituted by at least one hydroxy or an amino, preferably substituted on the pyrrolidinyl, especially in position 3,
   a (C₁-C₆)alkyloxy, preferably substituted on the phenyl, and
   a spirocycloalkyl, preferably a spirocyclopentyl, more preferably substituted on the pyrrolidinyl, especially in position 3,
R_{1′} represents a hydrogen or a (C₁-C₆)alkyl;
Y represents:
   a nitrogen, or
   a —CR₂ group in which R₂ is a radical selected in the group consisting of a hydrogen, a halogen, preferably a fluorine or a chlorine, and a (C₁-C₆)alkyloxy, preferably a methoxy;
R₃ represents a radical selected in the group consisting of:
   a 3-10 membered ring selected in the group consisting of a cycloalkyl, a heterocycloalkyl, and an aryl, said 3-10 membered ring is optionally substituted by a (C₁-C₆)alkyl optionally substituted by a (C₁-C₆)alkyloxy or a hydroxy, or a (C₁-C₆)alkyloxy,
   a (C₁-C₆)alkyl optionally substituted by at least one halogen,
   a (C₁-C₆)alkyloxy optionally substituted by at least one halogen, a (C₁-C₆)alkyloxy, or a —NH—CO₂—R₇ with R₇ being a (C₁-C₆)alkyl,
   a —NR₅R₆ with R₅ and R₆ being a (C₁-C₆)alkyl,
   a halogen,
   a hydrogen, and
   a cyano, or
R₃ may form with R₂ a dioxolanyl;
R_{3′} represents a hydrogen; and
R₄ represents a hydrogen or a (C₁-C₆)alkyloxy, preferably a methoxy.
A more preferred compound of formula (I) is such that:
R₁ represents an unsubstituted indolinyl, an indolinyl substituted by one radical selected in the group consisting of a halogen, preferably a fluorine, more preferably substituted on the phenyl, and a (C₁-C₆)alkyl, preferably a methyl or an isopropyl, more preferably substituted on the pyrrolidinyl, especially in position 3, or an indolinyl substituted by two (C₁-C₆)alkyls, preferably two methyls, more preferably substituted on the pyrrolidinyl, especially in position 3;
R_{1′} represents a hydrogen;
Y represents:
   a nitrogen, or
   a —CR₂ group in which R₂ is a radical selected in the group consisting of a hydrogen, a halogen, preferably a fluorine or a chlorine;
R₃ represents a radical selected in the group consisting of:
   a cyclopropyl, an azetidinyl, a pyrrolidinyl optionally substituted by a —CH₂—O—CH₃,
   a (C₁-C₆)alkyloxy, preferably a methoxy, a (C₁-C₆)alkyl, preferably a methyl, or an isopropyl,
   a —NR₅R₆ with R₅ and R₆ being a methyl,
   a (C₁-C₆)alkyl, preferably a methyl, and
   a halogen, preferably a chlorine;
R_{3′} represents a hydrogen; and
R₄ represents a hydrogen.

An even more preferred compound of formula (I) is selected in the group consisting of:
Example #99. 6-cyclopropyl-2-(indolin-1-ylmethyl)-3H-quinazolin-4-one;
Example #92. 6-(dimethylamino)-2-[(3,3-dimethylindolin-1-yl)methyl]-3H-pyrido[3,4-d]pyrimidin-4-one;
Example #77. 2-[(3,3-dimethylindolin-1-yl)methyl]-7-fluoro-6-methoxy-3H-quinazolin-4-one;
Example #93. 7-chloro-2-[(3,3-dimethylindolin-1-yl)methyl]-6-methoxy-3H-quinazolin-4-one;
Example #2. 2-[(3,3-dimethylindolin-1-yl)methyl]-6-methoxy-3H-quinazolin-4-one;
Example #4. 6-methoxy-2-[(3-methylindolin-1-yl)methyl]-3H-quinazolin-4-one, enantiomer 1;
Example #74. 6-(dimethylamino)-2-[(3,3-dimethylindolin-1-yl)methyl]-3H-quinazolin-4-one;
Example #6. 2-[(3-isopropylindolin-1-yl)methyl]-6-methoxy-3H-quinazolin-4-one;
Example #80. 6-(dimethylamino)-7-fluoro-2-(indolin-1-ylmethyl)-3H-quinazolin-4-one;
Example #100. 6-(azetidin-1-yl)-2-(indolin-1-ylmethyl)-3H-quinazolin-4-one;
Example #3. 6-methoxy-2-[(3-methylindolin-1-yl)methyl]-3H-quinazolin-4-one;
Example #1. 2-(indolin-1-ylmethyl)-6-methoxy-3H-quinazolin-4-one;
Example #73. 6-(dimethylamino)-2-(indolin-1-ylmethyl)-3H-quinazolin-4-one;
Example #81. 2-(indolin-1-ylmethyl)-6-isopropyl-3H-quinazolin-4-one;
Example #7. 2-[(6-fluoroindolin-1-yl)methyl]-6-methoxy-3H-quinazolin-4-one;
Example #76. 7-fluoro-2-(indolin-1-ylmethyl)-6-methoxy-3H-quinazolin-4-one;
Example #38. 6-chloro-2-[(3-methylindolin-1-yl)methyl]-3H-quinazolin-4-one enantiomer 1;
Example #79. 2-[(3,3-dimethylindolin-1-yl)methyl]-6-methoxy-3H-pyrido[3,4-d]pyrimidin-4-one;
Example #103. 2-(indolin-1-ylmethyl)-6-[3-(methoxymethyl)pyrrolidin-1-yl]-3H-quinazolin-4-one;
Example #43. 6-chloro-2-[(4-methylindolin-1-yl)methyl]-3H-quinazolin-4-one;
Example #82. 2-(indolin-1-ylmethyl)-6-(trifluoromethoxy)-3H-quinazolin-4-one;
Example #40. 6-chloro-2-[(3,3-dimethylindolin-1-yl)methyl]-3H-quinazolin-4-one;
Example #44. 6-chloro-2-[(7-methylindolin-1-yl)methyl]-3H-quinazolin-4-one;
Example #102. 2-(indolin-1-ylmethyl)-6-pyrrolidin-1-yl-3H-quinazolin-4-one;
Example #84. 2-(indolin-1-ylmethyl)-6-methyl-3H-quinazolin-4-one;
Example #37. 6-chloro-2-[(3-methylindolin-1-yl)methyl]-3H-quinazolin-4-one;
Example #78. 2-(indolin-1-ylmethyl)-6-methoxy-3H-pyrido[3,4-d]pyrimidin-4-one;
Example #5. 6-methoxy-2-[(3-methylindolin-1-yl)methyl]-3H-quinazolin-4-one, enantiomer 2;
Example #104. 6-[3-(hydroxymethyl)pyrrolidin-1-yl]-2-(indolin-1-ylmethyl)-3H-quinazolin-4-one;
Example #42. 6-chloro-2-(spiro[cyclopentane-1,3′-indoline]-1′-ylmethyl)-3H-quinazolin-4-one;
Example #85. 2-(indolin-1-ylmethyl)-6-(trifluoromethyl)-3H-quinazolin-4-one;
Example #86. 2-(indolin-1-ylmethyl)-7-methoxy-3H-quinazolin-4-one;
Example #87. 2-(indolin-1-ylmethyl)-6-isopropoxy-3H-quinazolin-4-one;
Example #47. 6-chloro-2-[(5-fluoroindolin-1-yl)methyl]-3H-quinazolin-4-one;
Example #45. 6-chloro-2-[(6-methylindolin-1-yl)methyl]-3H-quinazolin-4-one;
Example #88. 6-fluoro-2-(indolin-1-ylmethyl)-3H-quinazolin-4-one;
Example #46. 6-chloro-2-[(5-methylindolin-1-yl)methyl]-3H-quinazolin-4-one;
Example #94. 2-[(3,3-dimethylindolin-1-yl)methyl]-6,8-dimethoxy-3H-quinazolin-4-one;
Example #48. 2-[(5-bromoindolin-1-yl)methyl]-6-chloro-3H-quinazolin-4-one;
Example #10. 2-(isoindolin-2-ylmethyl)-6-methoxy-3H-quinazolin-4-one;
Example #119. 6-[(3,3-dimethylindolin-1-yl)methyl]-7H-[1,3]dioxolo[4,5-g]quinazolin-8-one;
Example #97. 2-[(3,3-dimethylindolin-1-yl)methyl]-6-(2-methoxyethoxy)-3H-quinazolin-4-one;
Example #120. 6-chloro-2-(1-indolin-1-ylethyl)-3H-quinazolin-4-one;
Example #8. 2-[(7-fluoroindolin-1-yl)methyl]-6-methoxy-3H-quinazolin-4-one;
Example #90. tert-butyl N-[4-[[2-(indolin-1-ylmethyl)-4-oxo-3H-quinazolin-6-yl]oxy]butyl]carbamate;
Example #41. 6-chloro-2-[(2-methylindolin-1-yl)methyl]-3H-quinazolin-4-one;
Example #91. 2-(indolin-1-ylmethyl)-7-(trifluoromethyl)-3H-quinazolin-4-one;
Example #101. 2-(indolin-1-ylmethyl)-6-(3-methoxyazetidin-1-yl)-3H-quinazolin-4-one;
Example #39. 6-chloro-2-[(3-methylindolin-1-yl)methyl]-3H-quinazolin-4-one enantiomer 2;
Example #96. 2-[(3,3-dimethylindolin-1-yl)methyl]-6-(3-methoxypropoxy)-3H-quinazolin-4-one;
Example #106. 2-(indolin-1-ylmethyl)-6-(1-piperidyl)-3H-quinazolin-4-one;
Example #49. 6-chloro-2-[(7-methoxyindolin-1-yl)methyl]-3H-quinazolin-4-one;
Example #95. 2-[(3,3-dimethylindolin-1-yl)methyl]-4-oxo-3H-quinazoline-6-carbonitrile;
Example #50. 6-chloro-2-(isoindolin-2-ylmethyl)-3H-quinazolin-4-one;
Example #105. 6-(3-hydroxypyrrolidin-1-yl)-2-(indolin-1-ylmethyl)-3H-quinazolin-4-one;
Example #129. 2-[[3-(2-hydroxyethyl)indolin-1-yl]methyl]-6-methoxy-3H-quinazolin-4-one;
Example #132. 2-(indolin-1-ylmethyl)-6-(4-methoxy-1-piperidyl)-3H-quinazolin-4-one;
Example #131. 2-[1-[(6-methoxy-4-oxo-3H-quinazolin-2-yl)methyl]indolin-3-yl]ethylammonium chloride;
Example #138. 7-fluoro-2-(indolin-1-ylmethyl)-3H-quinazolin-4-one;
Example #144. 6-chloro-2-((5-methoxyindolin-1-yl)methyl)quinazolin-4(3H)-one;
Example #126. 2-[(4-fluoroindolin-1-yl)methyl]-6-methoxy-3H-quinazolin-4-one; and
Example #9. 2-[[3-(hydroxymethyl)indolin-1-yl]methyl]-6-methoxy-3H-quinazolin-4-one.
   1,2,3,4-tetrahydroquinolinyl Series

In an embodiment, R₁ represents an optionally substituted 1,2,3,4-tetrahydroquinolinyl. In a further particular embodiment, R₁ represents an unsubstituted 1,2,3,4-tetrahydroquinolinyl or a 1,2,3,4-tetrahydroquinolinyl substituted by at least one radical as above defined.

In a preferred embodiment, R₁ represents an unsubstituted 1,2,3,4-tetrahydroquinolinyl or a 1,2,3,4-tetrahydroquinolinyl substituted by at least one radical selected in the group consisting of a halogen, preferably a fluorine or a chlorine, a (C₁-C₆)alkyl, preferably a methyl, a (C₁-C₆)alkyloxy, preferably a methoxy, and a —NH—SO₂(CH₃).

As above defined, a 1,2,3,4-tetrahydroquinolinyl corresponds to a phenyl fused to a piperidinyl. The 1,2,3,4-tetrahydroquinolinyl can be substituted on the phenyl and/or the piperidinyl by at least one radical as above defined. In a particular embodiment, the 1,2,3,4-tetrahydroquinolinyl is substituted on the phenyl by at least one radical selected from the group consisting of a halogen, preferably a a fluorine or a chlorine, a (C₁-C₆)alkyl, preferably a methyl, and a (C₁-C₆)alkyloxy, preferably a methoxy.

A preferred compound of formula (I) is such that:
R₁ represents an unsubstituted 1,2,3,4-tetrahydroquinolinyl or a 1,2,3,4-tetrahydroquinolinyl substituted by at least one radical, preferably on the phenyl, said radical is selected in the group consisting of a halogen, preferably a fluorine or a chlorine, a (C₁-C₆)alkyl, preferably a methyl, and a (C₁-C₆)alkyloxy, preferably a methoxy;
R_{1′} represents a hydrogen;
Y represents a —CR₂ group in which R₂ is a hydrogen;
R₃ represents a radical selected in the group consisting of a (C₁-C₆)alkyloxy, preferably a methoxy, and a halogen, preferably a chlorine;
R_{3′} represents a hydrogen; and
R₄ represents a hydrogen.

A more preferred compound of formula (I) is selected in the group consisting of:
Example #11. 2-[(8-fluoro-3,4-dihydro-2H-quinolin-1-yl)methyl]-6-methoxy-3H-quinazolin-4-one;
Example #12. 2-[(8-chloro-3,4-dihydro-2H-quinolin-1-yl)methyl]-6-methoxy-3H-quinazolin-4-one;
Example #13. 6-methoxy-2-[(8-methoxy-3,4-dihydro-2H-quinolin-1-yl)methyl]-3H-quinazolin-4-one;
Example #52. 6-chloro-2-[(5-methoxy-3,4-dihydro-2H-quinolin-1-yl)methyl]-3H-quinazolin-4-one;
Example #53. 6-chloro-2-[(5-methyl-3,4-dihydro-2H-quinolin-1-yl)methyl]-3H-quinazolin-4-one;
Example #54. 6-chloro-2-[(8-methoxy-3,4-dihydro-2H-quinolin-1-yl)methyl]-3H-quinazolin-4-one;
Example #15. 2-(3,4-dihydro-2H-quinolin-1-ylmethyl)-6-methoxy-3H-quinazolin-4-one;
Example #55. 6-chloro-2-[(7-methoxy-3,4-dihydro-2H-quinolin-1-yl)methyl]-3H-quinazolin-4-one; and
Example #127. 6-chloro-2-[(7-methyl-3,4-dihydro-2H-quinolin-1-yl)methyl]-3H-quinazolin-4-one.
   1,2,3,4-tetrahydroisoquinolinyl Series

In an embodiment, R₁ represents an optionally substituted 1,2,3,4-tetrahydroisoquinolinyl. In a further particular embodiment, R₁ represents an unsubstituted 1,2,3,4-tetrahydroisoquinolinyl or a 1,2,3,4-tetrahydroisoquinolinyl substituted by at least one radical as above defined.

In a preferred embodiment, R₁ represents an unsubstituted 1,2,3,4-tetrahydroisoquinolinyl or a 1,2,3,4-tetrahydroisoquinolinyl substituted by at least one radical selected in the group consisting of:
a halogen, preferably a bromine, a chlorine, and a fluorine,
a (C₁-C₆)alkyl optionally substituted by at least one a halogen, preferably a methyl or a trifluoromethyl,
a (C₁-C₆)alkyloxy optionally substituted by at least one hydroxy, an amino, or a halogen, preferably a methoxy,
a cyano,
a hydroxy,
a —NH—SO₂(CH₃), and
a —NR₅R₆ with R₅ and R₆ being a hydrogen.

In a more preferred embodiment, R₁ represents a 1,2,3,4-tetrahydroisoquinolinyl substituted by a radical selected in the group consisting of a cyano, a hydroxy, a (C₁-C₆)alkyloxy, preferably a methoxy, and a (C₁-C₆)alkyl, preferably a methyl, or a 1,2,3,4-tetrahydroisoquinolinyl substituted by one hydroxy and two (C₁-C₆)alkyloxys, preferably two methoxys.

As above defined, a 1,2,3,4-tetrahydroisoquinolinyl corresponds to a phenyl fused to a piperidinyl. The 1,2,3,4-tetrahydroquinolinyl can be substituted on the phenyl and/or the piperidinyl by at least one radical as above defined. In a particular embodiment, the 1,2,3,4-tetrahydroquinolinyl is substituted on the phenyl by at least one radical selected from the group consisting of a halogen, preferably a bromine, a chlorine, or a fluorine; a (C₁-C₆)alkyl optionally substituted by at least one a halogen, preferably a methyl or a trifluoromethyl; a (C₁-C₆)alkyloxy optionally substituted by at least a hydroxy, an amino, or a halogen, preferably a methoxy; a cyano; a hydroxy; a —NH—SO₂(CH₃), and a —NR₅R₆ with R₅ and R₆ being a hydrogen.

A preferred compound of formula (I) is such that:
R₁ represents an unsubstituted 1,2,3,4-tetrahydroisoquinolinyl or a 1,2,3,4-tetrahydroisoquinolinyl substituted, preferably on the phenyl, by at least one radical selected in the group consisting of:
   a halogen, preferably a bromine, a chlorine, and a fluorine,
   a (C₁-C₆)alkyl optionally substituted by at least one a halogen, preferably a methyl or a trifluromethyl,
   a (C₁-C₆)alkyloxy optionally substituted by at least one hydroxy, an amino, or a halogen, preferably a methoxy,
   a cyano,
   a hydroxy,
   a —NH—SO₂(CH₃), and
   a —NR₅R₆ with R₅ and R₆ being a hydrogen.
R_{1′} represents a hydrogen;
Y represents a —CR₂ group in which R₂ is a hydrogen;
R₃ represents a radical selected in the group consisting of a (C₁-C₆)alkyloxy, preferably a methoxy, and a halogen, preferably a chlorine;
R_{3′} represents a hydrogen; and
R₄ represents a hydrogen.
A more preferred compound of formula (I) is such that:
R₁ represents a 1,2,3,4-tetrahydroisoquinolinyl substituted, preferably on the phenyl, by a radical selected in the group consisting of a cyano, a hydroxy, a (C₁-C₆)alkyloxy, preferably a methoxy, and a (C₁-C₆)alkyl, preferably a methyl, or a 1,2,3,4-tetrahydroisoquinolinyl substituted by one hydroxy and two (C₁-C₆)alkyloxys, preferably two methoxys;
R_{1′} represents a hydrogen;
Y represents a —CR₂ group in which R₂ is a hydrogen;
R₃ represents a radical selected in the group consisting of a (C₁-C₆)alkyloxy, preferably a methoxy, and a halogen, preferably a chlorine;
R_{3′} represents a hydrogen; and
R₄ represents a hydrogen.

An even more preferred compound of formula (I) is selected in the group consisting of:
Example #16. 2-[(6-methoxy-4-oxo-3H-quinazolin-2-yl)methyl]-3,4-dihydro-1H-isoquinoline-5-carbonitrile;
Example #58. 6-chloro-2-[(5-methoxy-3,4-dihydro-1H-isoquinolin-2-yl)methyl]-3H-quinazolin-4-one;
Example #59A. 6-chloro-2-(1,2,3,4-tetrahydroisoquinolin-2-ium-2-ylmethyl)-3H-quinazolin-4-one; chloride
Example #17. 6-methoxy-2-[(5-methoxy-3,4-dihydro-1H-isoquinolin-2-yl)methyl]-3H-quinazolin-4-one;
Example #60. 6-chloro-2-[(7-methyl-3,4-dihydro-1H-isoquinolin-2-yl)methyl]-3H-quinazolin-4-one;
Example #18. 2-[(5-bromo-3,4-dihydro-1H-isoquinolin-2-yl)methyl]-6-methoxy-3H-quinazolin-4-one;
Example #19. 6-methoxy-2-[(8-methoxy-3,4-dihydro-1H-isoquinolin-2-yl)methyl]-3H-quinazolin-4-one;
Example #20. 2-[(5-chloro-3,4-dihydro-1H-isoquinolin-2-yl)methyl]-6-methoxy-3H-quinazolin-4-one;
Example #21. N-[2-[(6-methoxy-4-oxo-3H-quinazolin-2-yl)methyl]-3,4-dihydro-1H-isoquinolin-5-yl]methanesulfonamide;
Example #122. 2-(3,4-dihydro-1H-isoquinolin-2-ylmethyl)-6-methoxy-3H-quinazolin-4-one;
Example #123. 2-[(4-hydroxy-3,4-dihydro-1H-isoquinolin-2-yl)methyl]-6-methoxy-3H-quinazolin-4-one;
Example #124. 2-[(5-fluoro-3,4-dihydro-1H-isoquinolin-2-yl)methyl]-6-methoxy-3H-quinazolin-4-one;
Example #133. 2-[(5-amino-3,4-dihydro-1H-isoquinolin-2-yl)methyl]-6-methoxy-3H-quinazolin-4-one;
Example #130. 6-chloro-2-[(8-methoxy-3,4-dihydro-1H-isoquinolin-2-yl)methyl]-3H-quinazolin-4-one; and
Example #125. 6-methoxy-2-[[5-(trifluoromethyl)-3,4-dihydro-1H-isoquinolin-2-yl]methyl]-3H-quinazolin-4-one.
   2,3,4,5-tetrahydro-1,5-benzodiazepinyl and 2,3,4,5-tetrahydro-1-benzazepinyl Series

In an embodiment, R₁ represents an optionally substituted 2,3,4,5-tetrahydro-1,5-benzodiazepinyl or an optionally substituted 2,3,4,5-tetrahydro-1-benzazepinyl series. In a further particular embodiment, R₁ represents an unsubstituted 2,3,4,5-tetrahydro-1,5-benzodiazepinyl or a 2,3,4,5-tetrahydro-1,5-benzodiazepinyl substituted by at least one radical as defined above. In a further particular embodiment, R₁ represents an unsubstituted 2,3,4,5-tetrahydro-1-benzazepinyl or a 2,3,4,5-tetrahydro-1-benzazepinyl substituted by at least one radical as defined above.

In a preferred embodiment, R₁ represents an unsubstituted 2,3,4,5-tetrahydro-1-benzazepinyl or a 2,3,4,5-tetrahydro-1,5-benzodiazepinyl substituted by at least one (C₁-C₆)alkyl, preferably a methyl.

As defined above, a 2,3,4,5-tetrahydro-1-benzazepinyl corresponds to a phenyl fused to an azepinyl. A 2,3,4,5-tetrahydro-1,5-benzodiazepinyl corresponds to a phenyl fused to a diazepinyl. The 2,3,4,5-tetrahydro-1,5-benzodiazepinyl can be substituted on the phenyl and/or the diazepinyl by at least one radical as defined above.

In a particular embodiment, the 2,3,4,5-tetrahydro-1,5-benzodiazepinyl is substituted on the diazepinyl by at least one a (C₁-C₆)alkyl, preferably a methyl. In a preferred embodiment, the 2,3,4,5-tetrahydro-1,5-benzodiazepinyl is substituted on the diazepinyl by at least one a (C₁-C₆)alkyl, preferably a methyl, on the nitrogen atom.

A preferred compound of formula (I) is such that:
R₁ represents an unsubstituted 2,3,4,5-tetrahydro-1-benzazepinyl, or a 2,3,4,5-tetrahydro-1,5-benzodiazepinyl substituted, preferably on the diazepinyl, by a (C₁-C₆)alkyl, preferably a methyl,
R_{1′} represents a hydrogen;
Y represents a —CR₂ group in which R₂ is a hydrogen;
R₃ represents a radical selected in the group consisting of a (C₁-C₆)alkyloxy, preferably a methoxy, and a halogen, preferably a chlorine;
R₃ represents a hydrogen; and
R₄ represents a hydrogen.

A more preferred compound of formula (I) is selected in the group consisting of:
Example #22. 6-methoxy-2-(2,3,4,5-tetrahydro-1-benzazepin-1-ylmethyl)-3H-quinazolin-4-one;
Example #62. 6-chloro-2-(2,3,4,5-tetrahydro-1-benzazepin-1-ylmethyl)-3H-quinazolin-4-one;
Example #23. 6-methoxy-2-[(1-methyl-3,4-dihydro-2H-1,5-benzodiazepin-5-yl)methyl]-3H-quinazolin-4-one; and
Example #63. 6-chloro-2-[(1-methyl-3,4-dihydro-2H-1,5-benzodiazepin-5-yl)methyl]-3H-quinazolin-4-one.

In a particular aspect, the compound has an activity of ICD inducer. The activity can be determined by an assay measuring the Calreticulin release, the ATP secretion or the HMGB1 release. The assays are well-known by the person skilled in the art. The methods for measuring calreticulin release and HMGB1 release are detailed in the example section. In a very specific aspect, the compound has an EC50 of less than 20 μM in a method measuring the calreticulin release, in particular as detailed in example B.

The compounds of formula (I) according to the present invention can be prepared according to any chemical routes known from a skilled person, such as general synthetic routes presented in the examples. It is thus understood that one skilled in the art of organic chemistry can easily synthesize the compounds of formula (I) using appropriate starting materials, conventional chemicals reactions, standard and literatures procedures, and experimental conditions.

A particular aspect of the present invention relates to any pharmaceutically acceptable salt of the compounds as disclosed above and any enantiomer or diastereoisomer of the compounds as disclosed above.

A more particular aspect relates to any enantiomer of the compounds as disclosed above. These enantiomers can be obtained from the corresponding racemate which is purified by RP-HPLC chiral purifications methods (in particular the RP-HPLC chiral purification method of table 2) to give a first eluting enantiomer compound (so called “enantiomer 1”) and a second eluting enantiomer compound (so called “enantiomer 2”). The first enantiomer compound corresponds to the first compound eluted from the chiral RP-HPLC. The second enantiomer compound corresponds to the second compound eluted from the chiral RP-HPLC.

In a particular embodiment, the present invention relates to the following enantiomers:
Example #4, which is the enantiomer 1 of Example 3; and
Example #5, which is the enantiomer 2 of Example 3.

In an embodiment, the invention relates to the first enantiomer of Example #3. 6-methoxy-2-[(3-methylindolin-1-yl)methyl]-3H-quinazolin-4-one eluted from the chiral RP-HPLC purification method 4 of Table 2 with the following conditions: column: Chiralpack IA, 250×20 mm, 5 μm; Mobile phase: [cyclohexane 95%—EtOH 5%] isocratic; Flow rate: 9 mL/min; T° column: 25° C. Said first enantiomer of Example #3 is so-called Example #4. 6-methoxy-2-[(3-methylindolin-1-yl)methyl]-3H-quinazolin-4-one, enantiomer 1.

In a further embodiment, the invention relates to the second enantiomer of Example #3. 6-methoxy-2-[(3-methylindolin-1-yl)methyl]-3H-quinazolin-4-one eluted from the chiral RP-HPLC purification method 4 of Table 2 with the following conditions: column: Chiralpack IA, 250×20 mm, 1 μm; Mobile phase: [cyclohexane 95%—EtOH 5%] isocratic; Flow rate: 9 mL/min; T° column: 25° C. Said second enantiomer of Example #3 is so-called Example #5. 6-methoxy-2-[(3-methylindolin-1-yl)methyl]-3H-quinazolin-4-one, enantiomer 2.

In a further particular embodiment, the present invention relates to the following enantiomers:
Example #38, which is the enantiomer 1 of Example 37; and
Example #39, which is the enantiomer 2 of Example 37.

In an embodiment, the invention relates to the first enantiomer of Example #37. 6-chloro-2-[(3-methylindolin-1-yl)methyl]-3H-quinazolin-4-one eluted from the chiral RP-HPLC purification method 5 of Table 2 with the following conditions: column: Chiralpack IA, 250×20 mm, 5 μm; Mobile phase: [heptane 97%—EtOH 3%] isocratic; Flow rate: 18 mL/min; T° column: 25° C. Said first enantiomer of Example #37 is so-called Example #38. 6-chloro-2-[(3-methylindolin-1-yl)methyl]-3H-quinazolin-4-one enantiomer 1. In a further embodiment, the invention relates to the second enantiomer of Example #37. 6-chloro-2-[(3-methylindolin-1-yl)methyl]-3H-quinazolin-4-one eluted eluted from the chiral RP-HPLC purification method 5 of Table 2 with the following conditions: column: Chiralpack IA, 250×20 mm, 5 μm; Mobile phase: [heptane 97%—EtOH 3%]isocratic; Flow rate: 18 mL/min; T° column: 25° C. Said second enantiomer of Example #37 is so-called Example #39. 6-chloro-2-[(3-methylindolin-1-yl)methyl]-3H-quinazolin-4-one enantiomer 2.

### Therapeutic Applications

As illustrated by examples, the inventors have demonstrated the therapeutic interest of the compounds of the invention. Indeed, the inventors have shown that the compounds according to the present invention are capable of inducing ICD, more specifically have a Calreticulin EC50 lower than 12 μM, thereby demonstrating the therapeutic interest of such compounds in therapies, more particularly in cancer therapies. Therefore, the compounds of the present invention are useful as a drug.

Accordingly, the present invention relates to a compound as defined herein, for use as a drug or a medicine. The present invention further relates to a pharmaceutical or veterinary composition comprising a compound according to the invention. Preferably, the pharmaceutical composition further comprises a pharmaceutically or veterinary acceptable carrier or excipient. The present invention relates to the use of a compound according to the invention as a drug or a medicine. The invention further relates to a method for treating a disease in a subject, wherein a therapeutically effective amount of a compound according to the invention, is administered to said subject in need thereof. The invention also relates to the use of a compound according to the invention, for the manufacture of a medicine. The invention also relates to a pharmaceutical composition comprising a compound according to the invention for use as a drug.

The present invention also concerns:
a compound of formula (I) as defined above including anyone of the disclosed embodiments or a compound selected from the group consisting of 6-chloro-2-(indolin-1-ylmethyl)-3H-quinazolin-4-one, 6-chloro-2-(3,4-dihydro-2H-quinolin-1-ylmethyl)-3H-quinazolin-4-one, 6-chloro-2-(3,4-dihydro-1H-isoquinolin-2-ylmethyl)-3H-quinazolin-4-one, 6-chloro-2-[(4-hydroxy-5,8-dimethoxy-3,4-dihydro-1H-isoquinolin-2-yl)methyl]-3H-quinazolin-4-one, 2-(indolin-1-ylmethyl)-6,7-dimethoxy-3H-quinazolin-4-one and 7-chloro-2-(indolin-1-ylmethyl)-3H-quinazolin-4-one, or a pharmaceutical composition comprising such a compound for treating or for use for treating a cancer; and/or
a pharmaceutical composition comprising a compound of formula (I) as defined above including anyone of the disclosed embodiments or a compound selected from the group consisting of 6-chloro-2-(indolin-1-ylmethyl)-3H-quinazolin-4-one, 6-chloro-2-(3,4-dihydro-2H-quinolin-1-ylmethyl)-3H-quinazolin-4-one, 6-chloro-2-(3,4-dihydro-1H-isoquinolin-2-ylmethyl)-3H-quinazolin-4-one, 6-chloro-2-[(4-hydroxy-5,8-dimethoxy-3,4-dihydro-1H-isoquinolin-2-yl)methyl]-3H-quinazolin-4-one, 2-(indolin-1-ylmethyl)-6,7-dimethoxy-3H-quinazolin-4-one and 7-chloro-2-(indolin-1-ylmethyl)-3H-quinazolin-4-one, and an additional antitumor drug, for the treatment of cancer or for use in the treatment of cancer; and/or
a compound of formula (I) including anyone of the disclosed embodiments or a compound selected from the group consisting of 6-chloro-2-(indolin-1-ylmethyl)-3H-quinazolin-4-one, 6-chloro-2-(3,4-dihydro-2H-quinolin-1-ylmethyl)-3H-quinazolin-4-one, 6-chloro-2-(3,4-dihydro-1H-isoquinolin-2-ylmethyl)-3H-quinazolin-4-one, 6-chloro-2-[(4-hydroxy-5,8-dimethoxy-3,4-dihydro-1H-isoquinolin-2-yl)methyl]-3H-quinazolin-4-one, 2-(indolin-1-ylmethyl)-6,7-dimethoxy-3H-quinazolin-4-one and 7-chloro-2-(indolin-1-ylmethyl)-3H-quinazolin-4-one, or a pharmaceutical composition comprising such a compound, for treating cancer or for use for treating cancer in combination with radiotherapy, hyperthermia and/or other antitumor therapies, optionally before, simultaneously and/or after surgery (e.g., tumor resection); and/or
a kit comprising (a) a compound of formula (I) as defined above including anyone of the disclosed embodiments or a compound selected from the group consisting of 6-chloro-2-(indolin-1-ylmethyl)-3H-quinazolin-4-one, 6-chloro-2-(3,4-dihydro-2H-quinolin-1-ylmethyl)-3H-quinazolin-4-one, 6-chloro-2-(3,4-dihydro-1H-isoquinolin-2-ylmethyl)-3H-quinazolin-4-one, 6-chloro-2-[(4-hydroxy-5,8-dimethoxy-3,4-dihydro-1H-isoquinolin-2-yl)methyl]-3H-quinazolin-4-one, 2-(indolin-1-ylmethyl)-6,7-dimethoxy-3H-quinazolin-4-one and 7-chloro-2-(indolin-1-ylmethyl)-3H-quinazolin-4-one; and (b) an additional antitumor drug as a combined preparation for simultaneous, separate or sequential use, for treating cancer or for use for treating a cancer; and/or
the use of a compound of formula (I) as defined above including anyone of the disclosed embodiments or a compound selected from the group consisting of 6-chloro-2-(indolin-1-ylmethyl)-3H-quinazolin-4-one, 6-chloro-2-(3,4-dihydro-2H-quinolin-1-ylmethyl)-3H-quinazolin-4-one, 6-chloro-2-(3,4-dihydro-1H-isoquinolin-2-ylmethyl)-3H-quinazolin-4-one, 6-chloro-2-[(4-hydroxy-5,8-dimethoxy-3,4-dihydro-1H-isoquinolin-2-yl)methyl]-3H-quinazolin-4-one, 2-(indolin-1-ylmethyl)-6,7-dimethoxy-3H-quinazolin-4-one and 7-chloro-2-(indolin-1-ylmethyl)-3H-quinazolin-4-one, or a pharmaceutical composition comprising such a compound, for the manufacture of a medicament, a medicine or a drug for the treatment of a cancer; and/or
the use of a pharmaceutical composition comprising a compound of formula (I) as defined above including anyone of the disclosed embodiments or a compound selected from the group consisting of 6-chloro-2-(indolin-1-ylmethyl)-3H-quinazolin-4-one, 6-chloro-2-(3,4-dihydro-2H-quinolin-1-ylmethyl)-3H-quinazolin-4-one, 6-chloro-2-(3,4-dihydro-1H-isoquinolin-2-ylmethyl)-3H-quinazolin-4-one, 6-chloro-2-[(4-hydroxy-5,8-dimethoxy-3,4-dihydro-1H-isoquinolin-2-yl)methyl]-3H-quinazolin-4-one, 2-(indolin-1-ylmethyl)-6,7-dimethoxy-3H-quinazolin-4-one and 7-chloro-2-(indolin-1-ylmethyl)-3H-quinazolin-4-one, and an additional antitumor drug, for the manufacture of a medicament, a medicine or a drug for the treatment of a cancer; and/or
the use of a compound of formula (I) as defined above including anyone of the disclosed embodiments or a compound selected from the group consisting of 6-chloro-2-(indolin-1-ylmethyl)-3H-quinazolin-4-one, 6-chloro-2-(3,4-dihydro-2H-quinolin-1-ylmethyl)-3H-quinazolin-4-one, 6-chloro-2-(3,4-dihydro-1H-isoquinolin-2-ylmethyl)-3H-quinazolin-4-one, 6-chloro-2-[(4-hydroxy-5,8-dimethoxy-3,4-dihydro-1H-isoquinolin-2-yl)methyl]-3H-quinazolin-4-one, 2-(indolin-1-ylmethyl)-6,7-dimethoxy-3H-quinazolin-4-one and 7-chloro-2-(indolin-1-ylmethyl)-3H-quinazolin-4-one, or a pharmaceutical composition comprising such a compound, for the manufacture of a medicament, a medicine or a drug for the treatment of a cancer in combination with radiotherapy, hyperthermia and/or other antitumor therapies, optionally before, simultaneously and/or after surgery (e.g., tumor resection); and/or
a method for inducing ICD, in a subject in need thereof, comprising administering an effective amount of a compound of formula (I) as defined herein, or a compound selected from the group consisting of 6-chloro-2-(indolin-1-ylmethyl)-3H-quinazolin-4-one, 6-chloro-2-(3,4-dihydro-2H-quinolin-1-ylmethyl)-3H-quinazolin-4-one, 6-chloro-2-(3,4-dihydro-1H-isoquinolin-2-ylmethyl)-3H-quinazolin-4-one, 6-chloro-2-[(4-hydroxy-5,8-dimethoxy-3,4-dihydro-1H-isoquinolin-2-yl)methyl]-3H-quinazolin-4-one, 2-(indolin-1-ylmethyl)-6,7-dimethoxy-3H-quinazolin-4-one and 7-chloro-2-(indolin-1-ylmethyl)-3H-quinazolin-4-one, or a pharmaceutical composition comprising such a compound; in a particular aspect the subject has a cancer and the ICD induction by the compound promotes cancer treatment;
a method for treating a cancer, in a subject in need thereof, comprising administering an effective amount of a compound of formula (I) as defined herein, or a compound selected from the group consisting of 6-chloro-2-(indolin-1-ylmethyl)-3H-quinazolin-4-one, 6-chloro-2-(3,4-dihydro-2H-quinolin-1-ylmethyl)-3H-quinazolin-4-one, 6-chloro-2-(3,4-dihydro-1H-isoquinolin-2-ylmethyl)-3H-quinazolin-4-one, 6-chloro-2-[(4-hydroxy-5,8-dimethoxy-3,4-dihydro-1H-isoquinolin-2-yl)methyl]-3H-quinazolin-4-one, 2-(indolin-1-ylmethyl)-6,7-dimethoxy-3H-quinazolin-4-one and 7-chloro-2-(indolin-1-ylmethyl)-3H-quinazolin-4-one, or a pharmaceutical composition comprising such a compound or a pharmaceutical composition comprising such a compound;
a method for treating a cancer, in a subject in need thereof, comprising administering an effective amount of a compound of formula (I) as defined herein, or a compound selected from the group consisting of 6-chloro-2-(indolin-1-ylmethyl)-3H-quinazolin-4-one, 6-chloro-2-(3,4-dihydro-2H-quinolin-1-ylmethyl)-3H-quinazolin-4-one, 6-chloro-2-(3,4-dihydro-1H-isoquinolin-2-ylmethyl)-3H-quinazolin-4-one, 6-chloro-2-[(4-hydroxy-5,8-dimethoxy-3,4-dihydro-1H-isoquinolin-2-yl)methyl]-3H-quinazolin-4-one, 2-(indolin-1-ylmethyl)-6,7-dimethoxy-3H-quinazolin-4-one and 7-chloro-2-(indolin-1-ylmethyl)-3H-quinazolin-4-one, or a pharmaceutical composition comprising such a compound, and an additional antitumor drug;
a method for treating a cancer, in a subject in need thereof, comprising administering an effective amount of a compound of formula (I) as defined herein, or a compound selected from the group consisting of 6-chloro-2-(indolin-1-ylmethyl)-3H-quinazolin-4-one, 6-chloro-2-(3,4-dihydro-2H-quinolin-1-ylmethyl)-3H-quinazolin-4-one, 6-chloro-2-(3,4-dihydro-1H-isoquinolin-2-ylmethyl)-3H-quinazolin-4-one, 6-chloro-2-[(4-hydroxy-5,8-dimethoxy-3,4-dihydro-1H-isoquinolin-2-yl)methyl]-3H-quinazolin-4-one, 2-(indolin-1-ylmethyl)-6,7-dimethoxy-3H-quinazolin-4-one and 7-chloro-2-(indolin-1-ylmethyl)-3H-quinazolin-4-one, or a pharmaceutical composition comprising such a compound; the method further comprises radiotherapy, hyperthermia and/or other antitumor therapies, optionally before, simultaneously and/or after surgery (e.g., tumor resection).

The term “cancer”, as used herein, refers to the presence of cells possessing characteristics typical of cancer-causing cells, such as uncontrolled proliferation, immortality, metastatic potential, rapid growth and proliferation rate, and certain characteristic morphological features. The cancer may be solid tumor or hematopoietic tumor. Examples of cancer include, for example, leukemia, lymphoma, blastoma, carcinoma and sarcoma. More particular examples of such cancers include chronic myeloid leukemia, acute lymphoblastic leukemia, Philadelphia chromosome positive acute lymphoblastic leukemia (Ph+ALL), squamous cell carcinoma, lung cancer, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, osteosarcoma, pancreatic cancer, glioblastoma multiforme, cervical cancer, stomach cancer, bladder cancer, hepatoma, breast cancer, oesophagal cancer, colon carcinoma, and head and neck cancer, gastric cancer, germ cell tumor, pediatric sarcoma, sinonasal natural killer, multiple myeloma, acute myelogenous leukemia (AML), chronic lymphocytic leukemia, mastocytosis and any symptom associated with mastocytosis. In a particular aspect, the cancer is selected from the group consisting of colorectal, breast, ovarian, pancreatic, gastric, prostate, renal, cervical, myeloma, lymphoma, leukemia, thyroid, endometrial, uterine, bladder, neuroendocrine, head and neck, liver, nasopharyngeal, testicular, small cell lung cancer, non small cell lung cancer, melanoma, basal cell skin cancer, squamous cell skin cancer, dermatofibrosarcoma protuberans, Merkel cell carcinoma, glioblastoma, glioma, sarcoma, mesothelioma, and myelodisplastic syndromes.

The administration route can be topical, transdermal, oral, rectal, sublingual, intranasal, intrathecal, intratumoral or parenteral (including subcutaneous, intramuscular, intravenous and/or intradermal). Preferably, the administration route is parental, oral or topical. The pharmaceutical composition is adapted for one or several of the above-mentioned routes. The pharmaceutical composition, kit, product or combined preparation is preferably administered by injection or by intravenous infusion or suitable sterile solutions, or in the form of liquid or solid doses via the alimentary canal.

The pharmaceutical composition can be formulated as solutions in pharmaceutically compatible solvents or as emulsions, suspensions or dispersions in suitable pharmaceutical solvents or vehicles, or as pills, tablets or capsules that contain solid vehicles in a way known in the art. Formulations of the present invention suitable for oral administration may be in the form of discrete units as capsules, sachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion. Formulations for rectal administration may be in the form of a suppository incorporating the active ingredient and carrier such as cocoa butter, or in the form of an enema. Formulations suitable for parenteral administration conveniently comprise a sterile oily or aqueous preparation of the active ingredient which is preferably isotonic with the blood of the recipient. Every such formulation can also contain other pharmaceutically compatible and nontoxic auxiliary agents, such as, e.g. stabilizers, antioxidants, binders, dyes, emulsifiers or flavoring substances. The formulations of the present invention comprise an active ingredient in association with a pharmaceutically acceptable carrier therefore and optionally other therapeutic ingredients. The carrier must be “acceptable” in the sense of being compatible with the other ingredients of the formulations and not deleterious to the recipient thereof. The pharmaceutical compositions are advantageously applied by injection or intravenous infusion of suitable sterile solutions or as oral dosage by the digestive tract. Methods for the safe and effective administration of most of these chemotherapeutic agents are known to those skilled in the art. In addition, their administration is described in the standard literature.

Pharmaceutical compositions according to the invention may be formulated to release the active drug substantially immediately upon administration or at any predetermined time or time period after administration.

Preferably, the treatment with the compound according to the invention or the pharmaceutical composition according to the invention start no longer than a month, preferably no longer than a week, after the diagnosis of the disease. In a most preferred embodiment, the treatment starts the day of the diagnosis.

The compound according to the invention or the pharmaceutical composition according to the invention may be administered as a single dose or in multiple doses.

Preferably, the treatment is administered regularly, preferably between every day and every month, more preferably between every day and every two weeks, more preferably between every day and every week, even more preferably the treatment is administered every day. In a particular embodiment, the treatment is administered several times a day, preferably 2 or 3 times a day, even more preferably 3 times a day.

The duration of treatment with the compound according to the invention or the pharmaceutical composition according to the invention is preferably comprised between 1 day and 50 weeks, more preferably between 1 day and 30 weeks, still more preferably between 1 day and 15 weeks, even more preferably between 1 day and 10 weeks. In a particular embodiment, the duration of the treatment is of about 1 week. Alternatively, the treatment may last as long as the disease persists. The amount of compound according to the invention or of pharmaceutical composition according to the invention to be administered has to be determined by standard procedure well known by those of ordinary skills in the art. Physiological data of the patient (e.g. age, size, and weight) and the routes of administration have to be taken into account to determine the appropriate dosage, so as a therapeutically effective amount will be administered to the patient.

In a preferred embodiment, the total compound dose for each administration of the compound according to the invention or of the pharmaceutical composition according to the invention is comprised between 0.00001 and 1 g, preferably between 0.01 and 10 mg.

The form of the pharmaceutical compositions, the route of administration and the dose of administration of the compound according to the invention, or the pharmaceutical composition according to the invention can be adjusted by the man skilled in the art according to the type and severity of the disease, and to the patient, in particular its age, weight, sex, and general physical condition.

In one embodiment, the compound of the invention can be used in combination with another antitumoral drug or antineoplastic agent.

The additional antitumor drug can be selected in the non-exhaustive list of antitumor agents consisting of an inhibitor of topoisomerases I or II, an anti-mitotic agent, a DNA alkylating agent, an agent causing crosslinking of DNA, an anti-metabolic agent, a targeted agent such as a kinase inhibitor, a histone deacetylase inhibitor and an anti-EGFR agent and/or a therapeutical antibody designed to mediate cytotoxicity against the cancer cells or to modulate one of their key biological functions.

Antimitotic agents include, but are not limited to, Paclitaxel, Docetaxel and analogs such as Larotaxel (also called XRP9881; Sanofi-Aventis), XRP6258 (Sanofi-Aventis), BMS-184476 (Bristol-Meyer-Squibb), BMS-188797 (Bristol-Meyer-Squibb), BMS-275183 (Bristol-Meyer-Squibb), Ortataxel (also called IDN 5109, BAY 59-8862 or SB-T-101131; Bristol-Meyer-Squibb), RPR 109881A (Bristol-Meyer-Squibb), RPR 116258 (Bristol-Meyer-Squibb), NBT-287 (TAPESTRY), PG-Paclitaxel (also called CT-2103, PPX, Paclitaxel Poliglumex, Paclitaxel Polyglutamate or Xyotax™), ABRAXANE® (also called Nab-Paclitaxel; ABRAXIS BIOSCIENCE), Tesetaxel (also called DJ-927), IDN 5390 (INDENA), Taxoprexin (also called Docosahexanoic acid-Paclitaxel; PROTARGA), DHA-Paclitaxel (also called Taxoprexin®), and MAC-321 (WYETH). Preferably, antimitotic agents are Docetaxel, Paclitaxel, and is more preferably Docetaxel.

Inhibitors of topoisomerases I and/or II include, but are not limited to etoposide, topotecan, camptothecin, irinotecan, amsacrine, intoplicin, anthracyclines such as Doxorubicin, Epirubicin, Daunorubicin, Idarubicin and Mitoxantrone. Inhibitors of Topoisomerase I and II include, but are not limited to Intoplicin.

The additional antitumor agent can be alkylating agents including, without limitation, nitrogen mustards, ethylenimine derivatives, alkyl sulfonates, nitrosoureas, metal salts and triazenes. Non-exhaustive examples thereof include Uracil mustard, Chlormethine, Cyclophosphamide (CYTOXAN®), Ifosfamide, Melphalan, Chlorambucil, Pipobroman, Triethylenemelamine, Triethylenethiophosphoramine, Busulfan, Carmustine, Lomustine, Cisplatin, Carboplatin, Fotemustine, Oxaliplatin, Thiotepa, Streptozocin, Dacarbazine, and Temozolomide. In a preferred embodiment, the DNA alkylating agent is preferably Cisplatin, Fotemustine or Dacarbazine.

Anti-metabolic agents block the enzymes responsible for nucleic acid synthesis or become incorporated into DNA, which produces an incorrect genetic code and leads to apoptosis. Non-exhaustive examples thereof include, without limitation, folic acid antagonists, pyrimidine analogs, purine analogs and adenosine deaminase inhibitors, and more particularly Methotrexate, Floxuridine, Cytarabine, 6-Mercaptopurine, 6-Thioguanine, Fludarabine phosphate, Pentostatine, 5-Fluorouracil, Gemcitabine and Capecitabine.

The additional anti-tumor agent can also be a targeted agent, in particular a kinase inhibitor. The kinase may be selected from the group consisting of intracellular tyrosine or serine/threonine kinases, receptors tyrosine or serine/theonine kinase. The kinase could be selected among EGFR family, ALK, B-Raf, MEK, and mTOR. For instance, the agents may have ability to inhibit angiogenesis based on the inhibitory activities on VEGFR and PDGFR kinases. In particular, the targeted agent can be selected among the multiple kinase inhibitor drugs which are already approved: Gleevec, which inhibits Abl, and Iressa and Tarceva, which both inhibit EGFR, Sorafenib (Nexavar, BAY 43-9006) which inhibits Raf, Dasatinib (BMS-354825) and Nilotinib (AMN-107, Tasigna) which also inhibits Abl, Lapatinib which also inhibits EGFR, Temsirolimus (Torisel, CCI-779) which targets the mTOR pathway, Sunitinib (Stuten, SU11248) which inhibits several targets including VEGFR as well as specific antibodies inactivating kinase receptors: Herceptin and Avastin.

The anti-EGFR agent can be selected among gefitinib, erlotinib, lapatinib, vandetanib, afatinib, osimertinib, neratinib, dacomitinib, brigatinib, canertinib, naquotinib, nazartinib, pelitinib, rociletinib, icotinib, AZD3759, AZ5104 (CAS N2 1421373-98-9), poziotinib, WZ4002, preferably is Erlotinib or Cetuximab. The ALK inhibitor can be selected among crizotinib, entrectinib, ceritinib, alectinib, brigatinib, lorlatinib, TSR-011, CEP-37440, and ensartinib. The B-raf inhibitor can be selected among Vemurafenib, dabrafenib, regorafenib, and PLX4720. The MEK inhibitor can be selected among Cobimetinib, Trametinib, Binimetinib, Selumetinib, PD-325901, CI-1040, PD035901, U0126, TAK-733.

The additional drug can also be a checkpoint inhibitor, for instance an antibody targeting PD-1, PD-Li, CTLA-4 and the like.

Hyperthermia is a medical treatment in which is exposed to high temperatures to damage and kill cancer cells or to make cancer cells more sensitive to the effects of radiation and certain anticancer drugs. There are many techniques, well-known by the one skilled in the art, by which heat may be delivered. Some of the most common involve the use of focused ultrasound (FUS or HIFU), infrared sauna, microwave heating, induction heating, magnetic hyperthermia, infusion of warmed liquids, or direct application of heat such as through sitting in a hot room or wrapping a patient in hot blankets.

Further aspects and advantages of the invention will be disclosed in the following experimental section.

### EXAMPLES

### Example A—Chemistry

### Abbreviations

AcOH Acetic acid
ACN Acetonitrile
App Apparent
Aq Aqueous
br s Broad singlet
Cs₂CO₃ Cesium Carbonate
CDCl₃ Deuterated chloroform
d Doublet
DAD Diode Array Detector
DCM Dichloromethane (methylene chloride)
dd Doublet of doublets
ddd Doublet of doublets of doublets
DIAD N,N-Diisopropylazodicarboxylate
DIEA N,N-Diisopropylethylamine
DMF N,N-Dimethylformamide
DMSO Dimethyl sulfoxide
dt Doublet of triplets
eq Equivalent
EtOAc Ethyl acetate
Et₂O Diethyl ether
EtOH Ethanol
g Gram(s)
h Hour(s)
H₂SO₄ Sulfuric acid
HATU 1-[Bis(dimethylamino)methylene]-1H-1,2,3-triazolo[4,5-b]pyridinium 3-oxide hexafluorophosphate or Hexafluorophosphate
Azabenzotriazole Tetramethyl Uronium
HCl Hydrochloric acid
HCOOH Formic acid
HPLC High-pressure liquid chromatography
i-PrOH Isopropanol
K₂CO₃ Potassium carbonate
LC/MS Liquid chromatography/mass spectrometry
LiGH Lithium Hydroxide
m Multiplet
M Molar
m-CPBA 3-chloroperbenzoic acid
MeOH Methyl alcohol
MgSO₄ Magnesium sulfate
min Minute(s)
MHz MegaHertz
mg Milligram(s)
mL Millilitre(s)
mmol Millimole
mol/L Mole(s)/L
M Molar
MS Mass spectrometry
MsCl Methanesulfonyl chloride
MW Microwave
mp melting point
NH₄Cl Ammonium chloride
N Normal
NaH Sodium hydride
NaHCO₃ Sodium bicarbonate
NiCl₂, 6H₂O Nickel(II) chloride hexahydrate
NMR Nuclear magnetic resonance
p para
Pd₂(dba)₃ Bis(dibenzylidenacetone)palladium(0)
Pd(OAc)₂ Palladium acetate
PDA Photodiode Array
PPh₃ Triphenylphosphine
ppm Parts per million
q Quadruplet
quant Quantitative
quint Quintuplet
RP-HPLC Reverse-phase high-pressure liquid chromatography
Rt Retention time
rt Room temperature
s Singlet
S-Phos 2-dicyclohexylphosphino-2′,6′-dimethoxy-1,1′-biphenyl
X-Phos 2-Dicyclohexylphosphino-2′,4′,6′-triisopropylbiphenyl
t Triplet
tBuOK Potassium ter-butoxide
td Triplet of doublets
TEA Triethylamine
TFA Trifluoroacetic acid
tert-Tertiary
THF Tetrahydrofuran

General Synthetic Schemes

Compounds of the invention may be prepared using the synthetic transformations illustrated in Schemes I, V, VI, VII, and XIII. Starting materials are commercially available or may be prepared by the procedures described herein, by literature procedures, or by procedures that would be well known to one skilled in the art of organic chemistry. Unless stated, all aqueous solutions are saturated.

Methods for preparing 2-(aminomethyl)-3H-quinazolin-4-one compounds 5 of the invention are illustrated in Scheme I In Scheme I, step a, commercially available anthranilic acids 1 are reacted with chloroacetonitrile 2 using conditions described in Preparation #5, using an appropriate base and solvent such as NaOMe in MeOH as described in MedChemComm, 2014, 5(11), 1700-1707 or PCT Int. Appl. 2016, WO2016094730 or by methods known to one skilled in the art (for example, PCT Int. Appl. 2015, WO2015054572) to give 2-(chloromethyl)-3H-quinazolin-4-one intermediates 3. This intermediate 3, can undergo a nucleophilic substitution reaction with various cyclic amines 4 (Scheme I, step b) using conditions such as those described in Example #1 using an appropriate base and solvent such as K₂CO₃ in EtOH, or by methods known to one skilled in the art (for example European Journal of Medicinal Chemistry, 2012, 48, 231-243) to provide the 2-(aminomethyl)-3H-quinazolin-4-one compounds 5.

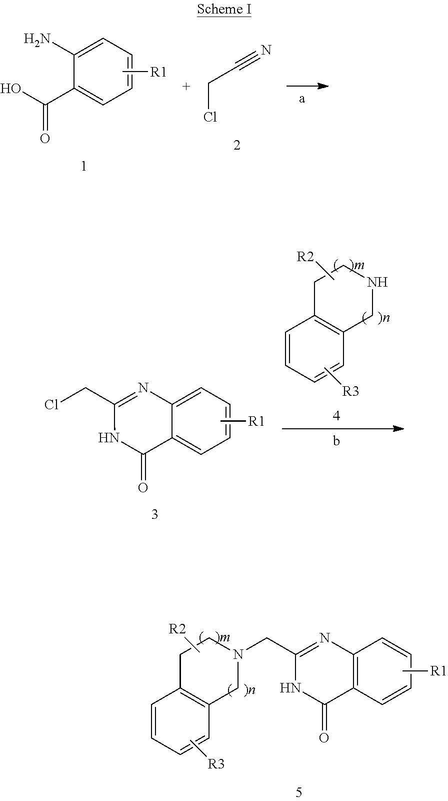

Substitution R1 on quinazolinone ring can be placed by functionalization of the anthranilic acid. If anthranilic acids 1 are commercially not available they can be prepared by methods known in the art and as described in literature, for example in Organic Preparations and Procedures International, 1981, 13(3-4), 189-96.

Substitution R1 on quinazolinone can also be placed by further functionalization of 2-(aminomethyl)-3H-quinazolin-4-one compound 5. Hence functionalization of phenol 14 can be performed using Mitsunobu reaction conditions with alcohol 15 in a manner similar to the one described in PCT Int. Appl. 2011, WO2011015526 to provide ether 16 (as exemplified in Scheme V).

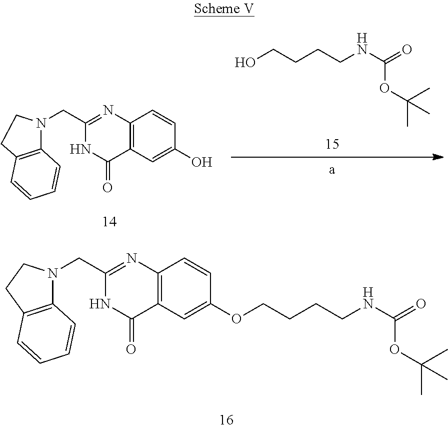 [SMILES: O=c1[nH]c(CN2CCc3ccccc32)nc2ccc(O)cc12 ; CC(C)(C)OC(=O)NCCCCO ; CC(C)(C)OC(=O)NCCCCOc1ccc2nc(CN3CCc4ccccc43)[nH]c(=O)c2c1]

In Scheme VI, step a, Substitution R1 on quinazolinone is introduced from bromo precursor such as exemplified by compound 17 using pallado-catalyzed reaction with the potassium salt of cyclopropyltrifluoroborate 18, Pd(OAc)₂ and S-Phos (see for example G. H. Fang, 2004, Org. Lett., 6, 357-360) to provide cyloalkyle product 19. In Scheme VI, step b, formation of aniline 21 is performed by pallado-catalyzed reaction between the bromoaryl substrate 17 with cyclic amines 20 in presence of tBuOK as base, X-Phos and Pd₂dba₃ as source of palladium or by methods known to one skilled in the art (see for example PCT Int. Appl. 2011, WO2011045258).

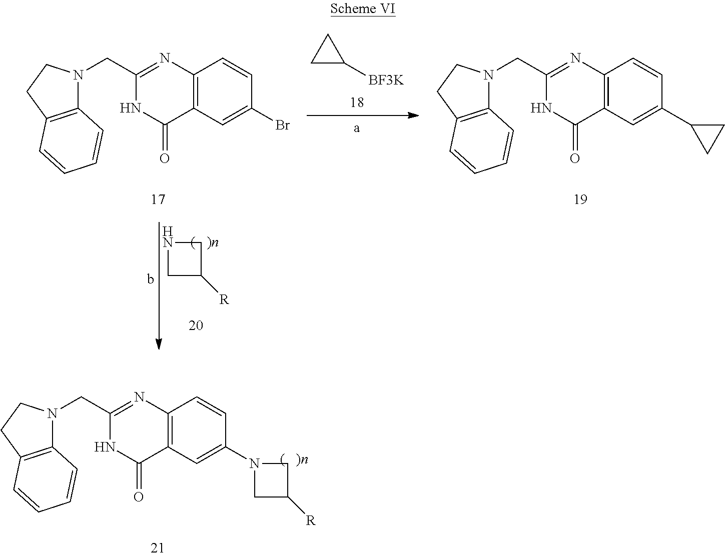

In scheme VII, heterocyclic bicyclic systems such as pyrido[3,4-d]pyrimidin-4-one 24 are prepared in similar manner a despicted in scheme I starting from aminoheterocyclic carboxylic acid 22 and converting them into chloromethylpyrido[3,4-d]pyrimidin-4-one 23 using chloroacetonitrile or by methods known to one skilled in the art (see for example U.S. Pat. Appl. 2016, US20160122343).

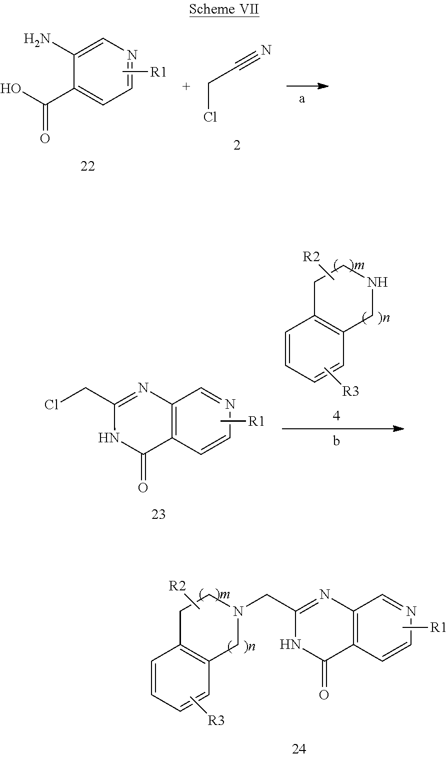

In Scheme XIII, introduction of substitution on methylene ring junction between the two heterocyclic systems can be performed with chloro substituted methylene 39 and nucleohilic substitution by amine 4 illustrated by Example #120 using similar conditions such as those described in Example #1 using an appropriate base and solvent such as K₂CO₃ in EtOH, or by methods known to one skilled in the art (for example European Journal of Medicinal Chemistry, 2012, 48, 231-243) to provide the methylene substituted product 40.

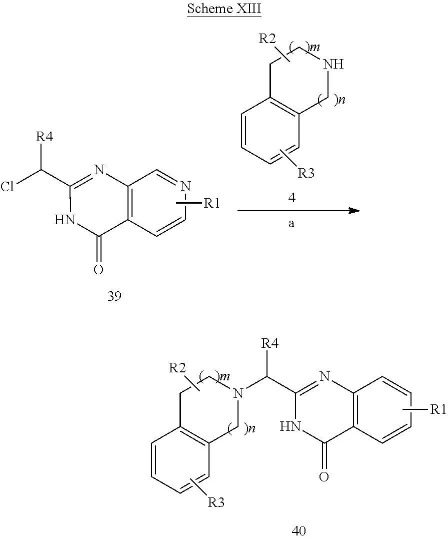

Analytical Methods

Analytical data is included within the procedures below, in the illustrations of the general procedures, or in the tables of examples. Unless otherwise stated, all ¹H NMR data were collected on a Bruker DPX 300 MHz equipped with 5 mm BB(O)F GRADZ probe, Bruker AVIII 400 MHz equipped with 5 mm BB(O)F GRADZ probe or Bruker AVIII 500 MHz equipped with 5 mm BBI GRADZ probe instruments and chemical shifts are quoted in parts per million (ppm). LC/MS was performed on HPLC Agilent 110 series instrument with a PDA detector from 1100 series coupled to Waters ZQ mass spectrometer, UPLC Acquity Waters with a PDA Acquity detector and a SQ Acquity mass spectrometer or a Shimadzu UFLC-XR system coupled to a LCMS-IT-TOF mass spectrometer. LC/MS data is referenced to LC/MS conditions using the method number provided in Table 1.

TABLE 1
LC/MS analysis methods

| Method | Conditions |
|---|---|
| A | LC/MS analysis condition: Column: KINETEX XB-C18 core- |
| | shell, 30 × 3 mm, 2.6 μm, Temperature 45° C., Mobile phase: |
| | ACN (0.1% AcOH) in water (0.1% AcOH), from 10% to 100% within 4 min; |
| | Flow rate: 1.4 ml/min; Wavelength: 210-260 nm DAD. |
| B | LC/MS analysis condition: Column: ACQUITY UPLC BEH C18, |
| | 50 × 2.1 mm, 1.7 μm, Temperature 45° C., Mobile phase: ACN (0.1% |
| | AcOH) in water (0.1% AcOH), from 5% to 95% within 2.50 min; Flow |
| | rate: 0.8 ml/min; Wavelength: 210-260 nm DAD. |
| C | LC/MS analysis condition: Column: XBRIDGE-C18, 50 × 2.1 mm, |
| | 2.6 μm, Temperature 45° C., Mobile phase: ACN (0.05% HCOOH) in |
| | water (0.1% HCOOH), from 5% to 95% within 4.9 min; Flow rate: 0.45 |
| | ml/min; Wavelength: 210-260 nm DAD. |
| D | LC/MS analysis condition: Column: Kinetex XBC18, 30 × 3 mm |
| | 2.6 μm, Temperature 45° C., Mobile phase: ACN (0.1% NH3) in water |
| | (0.1% NH3) from 5 to 95% within 4.5 min, Flow rate: 0.8 mL/min, |
| | Wavelength: 210-260 nm DAD. |
| E | LC/MS analysis condition: Column: KINETEX XB-C18 core- |
| | shell, 30 × 3 mm, 2.6 μm, Temperature 45° C., Mobile phase: |
| | ACN (0.1% AcOH) in water (0.1% AcOH), from 10% to 100% within 4 min; |
| | Flow rate: 1.4 ml/min; Wavelength: 210-260 nm DAD. |
| F | Normal Phase Chiral analytical condition: Column: Chiralpack IA, |
| | 250 × 4.6 mm, 5 μm, Mobile phase: cyclohexane 95% - EtOH 5%, isocratic, |
| | Flow rate: 1 mL/min, T° column: 25° C., Wavelength: 210 nm. |

Purification Methods

For the general procedures, intermediate and final compounds may be purified by any technique or combination of techniques known to one skilled in the art. Some examples that are not limiting include flash chromatography with a solid phase (i.e. silica gel, alumina, etc.) and a solvent (or combination of solvents, i.e. heptane, EtOAc, DCM, MeOH, ACN, water, etc.) that elutes the desired compounds; RP-HPLC purification performed on Waters system including a 2525 binary Pump, a ZQ Mass spectrometer, two 515 pumps, a PDA detector and a 2767 Autosampler, managed by MasslynX/fractionlynx (see Table 2 for some non-limiting conditions); recrystalization from an appropriate solvent (i.e. MeOH, EtOH, i-PrOH, EtOAc, toluene, etc.) or combination of solvents (i.e. EtOAc/heptane, EtOAc/MeOH, etc.); precipitation from a combination of solvents (i.e. DMF/water, DMSO/DCM, EtOAc/heptane, etc.); trituration with an appropriate solvent (i.e. EtOAc, DCM, ACN, MeOH, EtOH, i-PrOH, n-PrOH, etc.); extraction by dissolving a compound in a liquid and washing with an appropriately immiscible liquid (i.e. DCM/water, EtOAc/water, DCM/saturated NaHCO₃, EtOAc/saturated NaHCO₃, DCM/10% aqueous HCl, EtOAc/10% aqueous HCl, etc.); distillation (i.e. simple, fractional, Kugelrohr, etc.). Descriptions of these techniques can be found in the following references: Gordon, A. J. and Ford, R. A. “The Chemist's Companion”, 1972; Palleros, D. R. “Experimental Organic Chemistry”, 2000; Still, W. C., Kahn and M. Mitra, A. J. Org. Chem. 1978, 43, 2923; Yan, B. “Analysis and Purification Methods in Combinatorial Chemistry” 2003; Harwood, L. M., Moody, C. J. and Percy, J. M. “Experimental Organic Chemistry: Standard and Microscale, 2^{nd} Edition”, 1999.

TABLE 2
RP-HPLC and Chiral purification methods

| Method | Conditions |
|---|---|
| 1 | RP- HPLC purification condition: Column: KINETEX C18 |
| | 30 × 150 mm, 5 μm, Mobile phase: ACN in water (0.1% HCOOH); Flow |
| | rate: 42 ml/min; Wavelength: 210-260 nm DAD. Sample injected in |
| | DMSO/ACN. |
| 2 | RP- HPLC purification condition: Column: KINETEX C18 |
| | 30 × 150 mm, 5 μm, Mobile phase: ACN in water; Flow rate: 42 ml/min; |
| | Wavelength: 210-260 nm DAD. Sample injected in DMSO/ACN. |
| 3 | RP- HPLC purification condition: Column: X Bridge C18 30 × 150 |
| | mm, 5 μm, Mobile phase: ACN in water (0.1% AcOH); Flow rate: 42 |
| | ml/min; Wavelength: 210-260 nm DAD. Sample injected in DMSO/ACN. |
| 4 | Normal Phase Chiral analytical condition: Column: Chiralpack IA, |
| | 250 × 20 mm, 5 μm, Mobile phase: cyclohexane 95% - EtOH 5%, isocratic, |
| | Flow rate: 9 mL/min, T° column: 25° C., Wavelength: 210 nm, Sample |
| | preparation: 50/50 cyclohexane/isopropanol. |
| 5 | Normal Phase Chiral analytical condition: Column: Chiralpack IA, |
| | 250 × 20 mm, 5 μm, Mobile phase: Heptane 97% - EtOH 3%, isocratic, |
| | Flow rate: 18 mL/min, T° column: 25° C., Wavelength: 210 nm, Sample |
| | preparation: 80/20 cyclohexane/isopropanol |

### Preparation #5: 2-(chloromethyl)-6-methoxy-3H-pyrido[3,4-d]pyrimidin-4-one

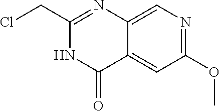

Chloroacetronitrile (CAS RN 107-14-2, 283 μL; 4.46 mmol; 3.00 eq.) was dissolved in MeOH (2.50 mL) and solution was cooled in an ice bath at 10° C. Sodium methoxide (16.1 mg; 0.30 mmol; 0.20 eq.) was added and the solution was stirred under N2 atm for 50 min at 10° C. A suspension of 5-amino-2-methoxy-4-pyridinecarboxylic acid (250 mg; 1.49 mmol; 1.00 eq.) in MeOH (5 mL) was added dropwise. Ice bath was removed and the mixture was stirred at room temperature overnight. The reaction mixture was diluted with 10 mL of H₂O and filtered. The solid was washed with H₂O three times and cold MeOH three times. Compound was dried under reduced pressure for 1 h at 50° C. and overnight at rt to give 2-(chloromethyl)-6-methoxy-3H-pyrido[3,4-d]pyrimidin-4-one (189 mg; 56%) as a beige amorphous powder. ¹H NMR (DMSO-d₆) δ: 12.70 (br s, 1H), 8.77 (d, J=0.7 Hz, 1H), 7.29 (d, J=0.7 Hz, 1H), 4.55 (s, 2H), 3.96 (s, 3H). LC/MS (Table 1, Method A) Rₜ=1.77 min m/z: 226 [M+H]+

### Preparation #6: 6-bromo-2-(indolin-1-ylmethyl)-3H-quinazolin-4-one

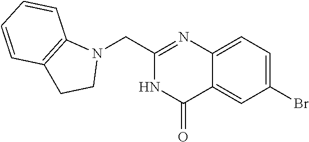 [SMILES: O=c1[nH]c(CN2CCc3ccccc32)nc2ccc(Br)cc12]

6-Bromo-2-(chloromethyl)-3H-quinazolin-4-one (CAS RN 177167-05-4, 6.00 g; 21.9 mmol; 1.00 eq.) was dissolved in EtOH (150 mL). K₂CO₃ (9.10 g; 65.9 mmol; 3.00 eq.) and indoline (CAS RN 496-15-1, 3.69 mL; 32.9 mmol; 1.50 eq.) were successively added. The mixture was stirred under N2 atm, at 80° C. overnight. The reaction was concentrated to dryness, then diluted in water and the solid formed was filtered. The solid was dried under reduce pressure at 50° C. for 18 h to give 6-bromo-2-(indolin-1-ylmethyl)-3H-quinazolin-4-one (7.39 g, 92%) as a beige solid. ¹H NMR (DMSO-d6) δ: 12.48 (br s, 1H), 8.18 (d, J=2.0 Hz, 1H), 7.94 (dd, J=2.0, 8.7 Hz, 1H), 7.60 (d, J=8.7 Hz, 1H), 7.06 (dd, J=0.7, 7.3 Hz, 1H), 6.98 (td, J=1.1, 7.7 Hz, 1H), 6.61 (td, J=1.1, 7.3 Hz, 1H), 6.58 (d, J=7.7 Hz, 1H), 4.20 (s, 2H), 3.50 (t, J=8.3 Hz, 2H), 2.93 (t, J=8.3 Hz, 2H). LC/MS (Table 1, Method A) Rₜ=2.63 min; MS m/z: 356 [M+H]+.

### Preparation #10: 6-chloro-2-(chloromethyl)quinazolin-4(3H)-one (CAS RN 2856-54-4)

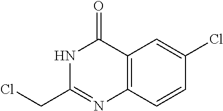 [SMILES: O=c1[nH]c(CCl)nc2ccc(Cl)cc12]

Sodium methoxide (30 wt % solution in MeOH) (1.513 ml, 8.17 mmol) was added dropwise to a solution of chloroacetonitrile (7.74 ml, 123 mmol) in MeOH (30 mL) cooled to 10° C. and the solution was stirred for 50 minutes at room temperature. A solution of 2-amino-5-chlorobenzoic acid (7.01 g, 40.9 mmol) in MeOH (100 mL) was then slowly added and the mixture was left to stir at room temperature for 3 h. The reaction mixture was then diluted with water 50 mL, and the resulting precipitate was filtered, washed with water (20 mL), cold MeOH (15 mL) and dried under reduced pressure to give 6-chloro-2-(chloromethyl)quinazolin-4(3H)-one (6.79 g, 72.6%) as a colourless powder. MS (ES+): 229/331 (M+H)+

### Preparation #11: 2-(chloromethyl)-6-(trifluoromethyl)quinazolin-4(3H)-one

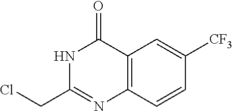 [SMILES: O=c1[nH]c(CCl)nc2ccc(C(F)(F)F)cc12]

A solution of 2-chloroacetonitrile (0.681 ml, 10.73 mmol) in MeOH (5 mL) at 0° C. under nitrogen was treated with sodium methoxide (30 wt % solution in MeOH) (0.133 ml, 0.716 mmol) dropwise. The resulting solution was allowed to warm to room temperature and stirred for 45 minutes. A solution of 2-amino-5-(trifluoromethyl)benzoic acid (0.734 g, 3.58 mmol) in MeOH (10 mL) was added slowly and the reaction was stirred at room temperature for 20 h. Water (30 mL) was added and the precipitate was collected by filtration, washing with water, then dried under vacuum at 50° C. to afford 2-(chloromethyl)-6-(trifluoromethyl)quinazolin-4(3H)-one (0.522 g, 55.0%) as a white solid. MS (ES+): 261 (M+H)+

### Example #1. 2-(indolin-1-ylmethyl)-6-methoxy-3H-quinazolin-4-one

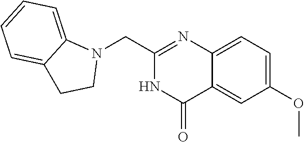 [SMILES: COc1ccc2nc(CN3CCc4ccccc43)[nH]c(=O)c2c1]

A mixture of 2-(chloromethyl)-6-methoxy-3H-quinazolin-4-one (CAS RN 1263413-60-0, 450 mg; 2.00 mmol; 1.00 eq.), indoline (CAS RN 496-15-1, 0.27 mL; 2.40 mmol; 1.20 eq.) and K₂CO₃ (415 mg; 3.00 mmol; 1.50 eq.) in EtOH (9 mL) was stirred at 80° C. overnight. Reaction mixture was diluted with H₂O and extracted with EtOAc three times. The combined organic layers were dried over MgSO₄, filtered and concentrated. The residue was purified by column chromatography (0 to 50% of EtOAc in cyclohexane) to give 2-(indolin-1-ylmethyl)-6-methoxy-3H-quinazolin-4-one (283 mg; 46%) as a white amorphous powder. ¹H NMR (400 MHz, DMSO-d₆) δ=12.24 (br s, 1H), 7.60 (d, J=8.9 Hz, 1H), 7.50 (d, J=3.0 Hz, 1H), 7.40 (dd, J=3.0, 8.9 Hz, 1H), 7.06 (d, J=7.3 Hz, 1H), 6.98 (dt, J=1.0, 7.7 Hz, 1H), 6.61 (dt, J=1.0, 7.3 Hz) 6.59 (d, J=7.7 Hz, 1H), 4.17 (s, 2H), 3.87 (s, 3H), 3.49 (t, J=8.3 Hz, 2H), 2.93 (t, J=8.3 Hz, 2H). LC/MS (Table 1, Method B) Rₜ=1.43 min; MS m/z: 308 [M+H]⁺.

### Example #2. 2-[(3,3-dimethylindolin-1-yl)methyl]-6-methoxy-3H-quinazolin-4-one

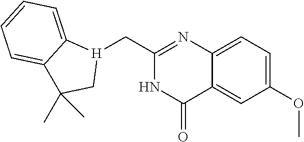

A mixture of 2-(chloromethyl)-6-methoxy-3H-quinazolin-4-one (CAS RN 1263413-60-0, 200 mg; 0.89 mmol; 1.00 eq.), 3,3-dimethylindoline (CAS RN 1914-02-9, 197 mg; 1.34 mmol; 1.50 eq.) and K₂CO₃ (369 mg; 2.67 mmol; 3.00 eq.) in EtOH (4 mL) was stirred at 80° C. overnight. After cooling down to rt, the suspension was filtered and washed with MeOH. The filtrate was concentrated to dryness. The crude was purified by column chromatography (5 to 30% of EtOAc in DCM) to give 2-[(3,3-dimethylindolin-1-yl)methyl]-6-methoxy-3H-quinazolin-4-one (203 mg; 55%) as a white amorphous powder. ¹H NMR (400 MHz, DMSO-d6) δ=12.27 (s, 1H), 7.59 (d, J=8.9 Hz, 1H), 7.50 (d, J=2.9 Hz, 1H), 7.40 (dd, J=2.9, 8.9 Hz, 1H), 7.02 (dd, J=0.9, 7.3 Hz, 1H), 6.99 (dt, J=1.2, 7.7 Hz, 1H), 6.64 (dt, J=0.9, 7.3 Hz), 6.61 (d, J=7.7 Hz, 1H), 4.17 (s, 2H), 3.87 (s, 3H), 3.27 (s, 2H), 1.25 (s, 6H). LC/MS (Table 1, Method B) Rₜ=1.65 min; MS m/z: 336 [M+H]⁺.

### Example #3. 6-methoxy-2-[(3-methylindolin-1-yl)methyl]-3H-quinazolin-4-one

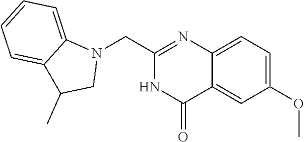 [SMILES: COc1ccc2nc(CN3CC(C)c4ccccc43)[nH]c(=O)c2c1]

The compound was synthesized using the same procedure detailed in Example #2 starting from 2-(chloromethyl)-6-methoxy-3H-quinazolin-4-one (CAS RN 1263413-60-0, 200 mg; 0.89 mmol; 1.00 eq.) and 3-methyl-2,3-dihydro-1H-indole hydrochloride (CAS RN 4375-15-9, 197 mg; 1.16 mmol; 1.30 eq.). The residue was purified by column chromatography (0 to 65% of EtOAc in cyclohexane) giving 6-methoxy-2-[(3-methylindolin-1-yl)methyl]-3H-quinazolin-4-one (175 mg; 61%) as a white amorphous powder. mp=192-213° C. ¹H NMR (400 MHz, DMSO-d₆) δ 12.24 (s, 1H), 7.60 (d, J=8.9 Hz, 1H), 7.50 (d, J=2.9 Hz, 1H), 7.40 (dd, J=2.9, 8.9 Hz, 1H), 7.05 (d, J=7.4 Hz, 1H), 6.99 (dt, J=0.9, 7.8 Hz, 1H), 6.64 (dt, J=0.9, 7.4 Hz, 1H), 6.60 (d, J=7.8 Hz, 1H), 4.25 (d, J=15.1 Hz, 1H), 4.05 (d, J=15.1 Hz, 1H), 3.87 (s, 3H), 3.71 (t, J=8.6 Hz, 1H), 3.30-3.19 (m, 1H), 2.99 (t, J=8.6 Hz, 1H), 1.26 (d, J=6.82 Hz, 3H). LC/MS (Table 1, Method B) Rₜ=1.55 min; MS m/z: 322 [M+H]⁺.

### Example #4. 6-methoxy-2-[(3-methylindolin-1-yl)methyl]-3H-quinazolin-4-one, Enantiomer 1

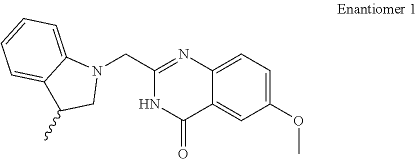 [SMILES: COc1ccc2nc(CN3CC(C)c4ccccc43)[nH]c(=O)c2c1]

6-Methoxy-2-[(3-methylindolin-1-yl)methyl]-3H-quinazolin-4-one (Example #3) was separated by chiral LC-MS (Table 2, Condition 4) (column: Chiralpack IA, 250×20 mm, 5 μm, Mobile phase: [cyclohexane 95%—EtOH 5%] isocratic, Flow rate: 9 mL/min, T° column: 25° C.) giving 6-methoxy-2-[(3-methylindolin-1-yl)methyl]-3H-quinazolin-4-one, enantiomer I (ee>99%) (46 mg; 16%) as a white amorphous powder. ¹H NMR (400 MHz, DMSO-d₆) δ=12.24 (br s, 1H), 7.59 (d, J=8.9 Hz, 1H), 7.50 (d, J=3.0 Hz, 1H), 7.40 (dd, J=3.0, 8.9 Hz, 1H), 7.05 (d, J=7.2 Hz, 1H), 6.99 (dt, J=0.8, 7.7 Hz, 1H), 6.64 (dt, J=0.8, 7.2 Hz, 1H), 6.60 (d, J=7.7 Hz, 1H), 4.25 (d, J=15.1 Hz, 1H), 4.05 (d, J=15.1 Hz, 1H), 3.86 (s, 3H), 3.71 (t, J=8.6 Hz, 1H), 3.30-3.20 (m, 1H), 2.99 (t, J=8.6 Hz, 1H), 1.26 (d, J=6.7 Hz, 3H). LC/MS (Table 1, Method B) Rₜ=1.56 min; MS m/z: 322 [M+H]⁺. LC/MS (Table 1, Method E) Rₜ=25.1 min;

### Example #5. 6-methoxy-2-[(3-methylindolin-1-yl)methyl]-3H-quinazolin-4-one, Enantiomer 2

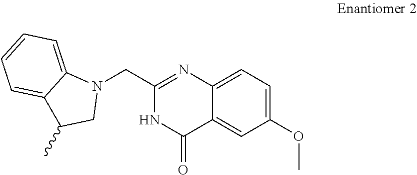 [SMILES: COc1ccc2nc(CN3CC(C)c4ccccc43)[nH]c(=O)c2c1]

6-Methoxy-2-[(3-methylindolin-1-yl)methyl]-3H-quinazolin-4-one (Example #3) was separated by chiral LC-MS (Table 2, Condition 4) (column: Chiralpack IA, 250×20 mm, 5 μm, Mobile phase: [cyclohexane 95%—EtOH 5%], Flow rate: 9 mL/min, T° column: 25° C.) giving 6-methoxy-2-[(3-methylindolin-1-yl)methyl]-3H-quinazolin-4-one, enantiomer 2 (ee>99%) (48 mg; 16.8%) as a white amorphous powder. ¹H NMR (400 MHz, DMSO-d₆) δ=12.24 (br s, 1H), 7.59 (d, J=8.9 Hz, 1H), 7.50 (d, J=3.0 Hz, 1H), 7.40 (dd, J=3.0, 8.9 Hz, 1H), 7.05 (d, J=7.2 Hz, 1H), 6.99 (t, J=7.7 Hz, 1H), 6.64 (dt, J=0.8, 7.2 Hz, 1H), 6.60 (d, J=7.7 Hz, 1H), 4.25 (d, J=15.1 Hz, 1H), 4.05 (d, J=15.1 Hz, 1H), 3.86 (s, 3H), 3.71 (t, J=8.6 Hz, 1H), 3.30-3.20 (m, 1H), 2.99 (t, J=8.6 Hz, 1H), 1.26 (d, J=6.7 Hz, 3H). LC/MS (Table 1, Method B) Rₜ=1.55 min; MS m/z: 322 [M+H]⁺. LC/MS (Table 1, Method E) Rₜ=27.7 min

### Example #6. 2-[(3-isopropylindolin-1-yl)methyl]-6-methoxy-3H-quinazolin-4-one

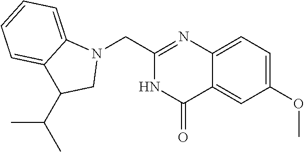 [SMILES: COc1ccc2nc(CN3CC(C(C)C)c4ccccc43)[nH]c(=O)c2c1]

The compound was synthesized using the same procedure detailed in Example #1 starting 2-(chloromethyl)-6-methoxy-3H-quinazolin-4-one (CAS RN 1263413-60-0, 150 mg; 0.67 mmol; 1.00 eq.) and 3-isopropylindoline (CAS RN 67932-69-8, 134 μL; 0.80 mmol; 1.20 eq.). The residue was purified by column chromatography (0 to 40% of EtOAc in cyclohexane) giving 2-[(3-isopropylindolin-1-yl)methyl]-6-methoxy-3H-quinazolin-4-one (138 mg; 59%) as a white amorphous powder. mp=192-208° C. ¹H NMR (400 MHz, DMSO-d₆) δ=12.19 (br s, 1H), 7.57 (d, J=8.9 Hz, 1H), 7.49 (d, J=3.0 Hz, 1H), 7.40 (dd, J=3.0, 8.9 Hz, 1H), 7.04 (d, J=7.4 Hz, 1H), 6.98 (t, J=7.8 Hz, 1H), 6.60 (td, J=0.9, 7.4 Hz, 1H), 7.57 (d, J=7.8 Hz, 1H), 4.17 (s, 2H), 3.86 (s, 3H), 3.53 (t, J=9.0 Hz, 1H), 3.37 (dd, J=6.8, 9.0 Hz, 1H), 3.20-3.12 (m, 1H), 2.09-1.97 (m, 1H), 0.93 (d, J=6.8 Hz, 3H), 0.82 (d, J=6.8 Hz, 3H). LC/MS (Table 1, Method B) Rₜ=1.77 min; MS m/z: 350 [M+H]⁺.

### Example #7. 2-[(6-fluoroindolin-1-yl)methyl]-6-methoxy-3H-quinazolin-4-one

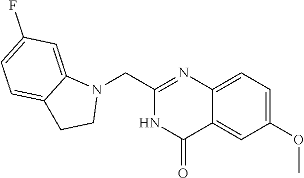 [SMILES: COc1ccc2nc(CN3CCc4ccc(F)cc43)[nH]c(=O)c2c1]

The compound was synthesized using the same procedure detailed in Example #2 starting from 2-(chloromethyl)-6-methoxy-3H-quinazolin-4-one (CAS RN 1263413-60-0, 200 mg; 0.89 mmol; 1.00 eq.) and 6-fluoroindoline (CAS RN 2343-23-9, 183 mg; 1.34 mmol; 1.50 eq.). The 5 residue was purified by column chromatography (0 to 30% of EtOAc in DCM) giving 2-[(6-fluoroindolin-1-yl)methyl]-6-methoxy-3H-quinazolin-4-one (168 mg; 58%) as a white amorphous solid. mp=229-235° C. ¹H NMR (400 MHz, DMSO-d₆) δ=12.27 (br s, 1H), 7.59 (d, J=8.8 Hz, 1H), 7.50 (d, J=3.0 Hz, 1H), 7.40 (dd, J=3.0, 8.8 Hz, 1H), 7.03-6.97 (m, 1H), 6.46 (dd, J=2.4, 10.8 Hz, 1H), 6.33 (ddd, J=2.4, 7.8, 9.9 Hz, 1H), 4.21 (s, 2H), 3.87 (s, 3H), 3.57 (t, J=8.4 Hz, 2H), 2.90 (t, J=8.4 Hz, 2H). LC/MS (Table 1, Method B) Rₜ=1.48 min; MS m/z: 326 [M+H]⁺.

### Example #8. 2-[(7-fluoroindolin-1-yl)methyl]-6-methoxy-3H-quinazolin-4-one

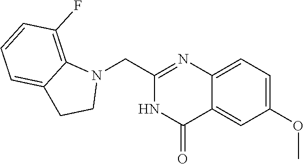 [SMILES: COc1ccc2nc(CN3CCc4cccc(F)c43)[nH]c(=O)c2c1]

The compound was synthesized using the same procedure detailed in Example #1 starting from 2-(chloromethyl)-6-methoxy-3H-quinazolin-4-one (CAS RN 1263413-60-0, 150 mg; 0.67 mmol; 1.00 eq.) and 7-fluoroindoline (CAS RN 769966-04-3, 110 mg; 0.80 mmol; 1.20 eq.). The residue was purified by column chromatography (0 to 40% of EtOAc in cyclohexane) giving 2-[(7-fluoroindolin-1-yl)methyl]-6-methoxy-3H-quinazolin-4-one (103 mg; 47%) as a white amorphous solid. mp=208-227° C. ¹H NMR (400 MHz, DMSO-d₆) δ=12.19 (br s, 1H), 7.49 (d, J=8.9 Hz, 1H), 7.48 (d, J=3.0 Hz, 1H), 7.36 (dd, J=3.0, 8.9 Hz, 1H), 6.91 (dd, J=0.9, 7.3 Hz, 1H), 6.82 (ddd, J=0.9, 8.0 Hz, 1H), 6.60 (ddd, J=4.5, 7.3, 8.0 Hz, 1H), 4.41 (s, 2H), 3.86 (s, 3H), 3.56 (t, J=8.6 Hz, 2H), 3.02 (t, J=8.6 Hz, 2H). LC/MS (Table 1, Method B) Rₜ=1.45 min; MS m/z: 326 [M+H]⁺.

### Example #9. 2-[[3-(hydroxymethyl)indolin-1-yl]methyl]-6-methoxy-3H-quinazolin-4-one

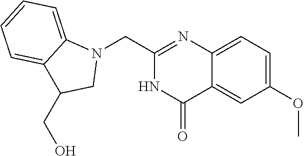 [SMILES: COc1ccc2nc(CN3CC(CO)c4ccccc43)[nH]c(=O)c2c1]

The compound was synthesized using the same procedure detailed in Example #2 starting from 2-(chloromethyl)-6-methoxy-3H-quinazolin-4-one (CAS RN 1263413-60-0, 100 mg; 0.45 mmol; 1.00 eq.) and indolin-3-ylmethanol (CAS RN 936829-06-0, 100 mg; 0.67 mmol; 1.50 eq.). The residue was purified by column chromatography (10 to 80% of EtOAc in DCM) giving 2-[[3-(hydroxymethyl)indolin-1-yl]methyl]-6-methoxy-3H-quinazolin-4-one (52 mg; 32%) as a beige amorphous powder. mp=190-209° C. ¹H NMR (400 MHz, DMSO-d₆) δ=12.21 (br s, 1H), 7.59 (d, J=8.9 Hz, 1H), 7.50 (d, J=3.0 Hz, 1H), 7.40 (dd, J=3.0, 8.9 Hz, 1H), 7.09 (d, J=7.3 Hz, 1H), 6.99 (dt, J=0.9, 7.8 Hz, 1H), 6.60 (td, J=0.9, 7.3 Hz, 1H) 6.58 (d, J=7.8 Hz, 1H), 4.80 (t, J=5.2 Hz, 1H), 4.18 (d, J=15.2 Hz, 1H), 4.17 (d, J=15.2 Hz, 1H), 3.87 (s, 3H), 3.69-3.61 (m, 1H), 3.61 (m, 1H), 3.47 (m, 1H), 3.37-3.33 (m, 1H), 3.32-3.27 (m, 1H). LC/MS (Table 1, Method B) Rₜ=120 min; MS m/z: 338 [M+H]⁺.

### Example #10. 2-(isoindolin-2-ylmethyl)-6-methoxy-3H-quinazolin-4-one

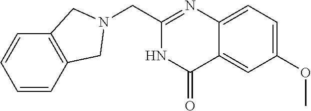 [SMILES: COc1ccc2nc(CN3Cc4ccccc4C3)[nH]c(=O)c2c1]

The compound was synthesized using the same procedure detailed in Example #2 starting from 2-(chloromethyl)-6-methoxy-3H-quinazolin-4-one (CAS RN 1263413-60-0, 200 mg; 0.89 mmol; 1.00 eq.) and isoindoline (CAS RN 496-12-8, 200 μL; 1.78 mmol; 2.00 eq.). The residue was purified by column chromatography (0 to 50% of EtOAc in cyclohexane) giving 2-(isoindolin-2-ylmethyl)-6-methoxy-3H-quinazolin-4-one (90 mg; 31.48%) as a beige amorphous powder. ¹H NMR (400 MHz, DMSO-d₆) δ=12.07 (br s, 1H), 7.62 (d, J=8.9 Hz, 1H), 7.50 (d, J=3.0 Hz, 1H), 7.41 (dd, J=3.0, 8.9 Hz, 1H), 7.27-7.22 (m, 2H), 7.22-7.16 (m, 2H), 4.00 (s, 4H), 3.87 (s, 3H), 3.82 (s, 2H). LC/MS (Table 1, Method B) Rₜ=0.86 min; MS m/z: 308 [M+H]⁺.

### Example #11. 2-[(8-fluoro-3,4-dihydro-2H-quinolin-1-yl)methyl]-6-methoxy-3H-quinazolin-4-one

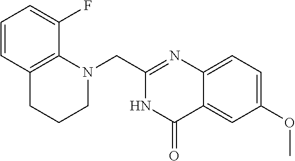 [SMILES: COc1ccc2nc(CN3CCCc4cccc(F)c43)[nH]c(=O)c2c1]

The compound was synthesized using the same procedure detailed in Example #2 starting from 2-(chloromethyl)-6-methoxy-3H-quinazolin-4-one (CAS RN 1263413-60-0, 100 mg; 0.45 mmol; 1.00 eq.) and 8-fluoro-1,2,3,4-tetrahydroquinoline (CAS RN 75414-02-7, 168 mg; 1.11 mmol; 2.50 eq.). The residue was purified by column chromatography (10 to 50% of EtOAc in DCM) to give 2-[(8-fluoro-3,4-dihydro-2H-quinolin-1-yl)methyl]-6-methoxy-3H-quinazolin-4-one (50 mg; 32%) as a white amorphous powder. mp=222-236° C. ¹H NMR (DMSO-d₆) δ: 11.98 (br s, 1H), 7.49 (d, J=8.8 Hz, 1H), 7.48 (d, J=3.0 Hz, 1H), 7.37 (dd, J=3.0, 8.8 Hz, 1H), 6.83 (dt, J=1.3, 8.0 Hz, 1H), 6.81 (d, J=6.3 Hz, 1H), 6.63 (td, J=7.8, 4.8 Hz, 1H), 4.37 (d, J=2.2 Hz, 2H), 3.86 (s, 3H), 3.34 (t, J=5.6 Hz, 2H), 2.75 (t, J=6.3 Hz, 2H), 1.83-1.91 (m, 2H). LC/MS (Table 1, Method B) Rₜ=1.54 min; MS m/z: 340 [M+H]⁺.

### Example #12. 2-[(8-chloro-3,4-dihydro-2H-quinolin-1-yl)methyl]-6-methoxy-3H-quinazolin-4-one

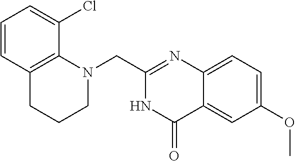 [SMILES: COc1ccc2nc(CN3CCCc4cccc(Cl)c43)[nH]c(=O)c2c1]

The compound was synthesized using the same procedure detailed in Example #2 starting from 2-(chloromethyl)-6-methoxy-3H-quinazolin-4-one (CAS RN 1263413-60-0, 100 mg; 0.45 mmol; 1.00 eq.) and 8-chloro-1,2,3,4-tetrahydroquinoline (CAS RN 90562-36-0, 112 mg; 0.67 mmol; 1.50 eq.). The residue was purified by column chromatography (0 to 20% of EtOAc in DCM) giving 2-[(8-chloro-3,4-dihydro-2H-quinolin-1-yl)methyl]-6-methoxy-3H-quinazolin-4-one (8 mg; 5%) as white amorphous powder. ¹H NMR (DMSO-d₆) δ: 11.66 (br s, 1H), 7.58 (d, J=8.9 Hz, 1H), 7.51 (d, J=3.0 Hz, 1H), 7.41 (dd, J=3.0, 8.9 Hz, 1H), 7.21 (dd, J=1.5, 7.7 Hz, 1H), 7.05 (dd, J=1.4, 7.7 Hz, 1H), 6.89 (t, J=7.7 Hz, 1H), 4.21 (s, 2H), 3.87 (s, 3H), 3.14-3.21 (m, 2H), 2.80 (t, J=6.5 Hz, 2H), 1.76-1.84 (m, 2H). LC/MS (Table 1, Method B) Rₜ=1.63 min; MS m/z: 356 [M+H]⁺.

### Example #13. 6-methoxy-2-[(8-methoxy-3,4-dihydro-2H-quinolin-1-yl)methyl]-3H-quinazolin-4-one

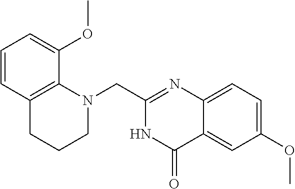 [SMILES: COc1ccc2nc(CN3CCCc4cccc(OC)c43)[nH]c(=O)c2c1]

The compound was synthesized using the same procedure detailed in Example #2 starting from 2-(chloromethyl)-6-methoxy-3H-quinazolin-4-one (CAS RN 1263413-60-0, 100 mg; 0.45 mmol; 1.00 eq.) and 8-methoxy-1,2,3,4-tetrahydroquinoline (CAS RN 53899-17-5, 109 mg; 0.67 mmol; 1.50 eq.). The residue was purified by column chromatography (0 to 30% of EtOAc in cyclohexane) giving 6-methoxy-2-[(8-methoxy-3,4-dihydro-2H-quinolin-1-yl)methyl]-3H-quinazolin-4-one (76 mg; 49%) as a beige amorphous powder. mp=169-183° C. 1H NMR (DMSO-d₆) δ: 11.10 (s, 1H), 7.59 (d, J=8.9 Hz, 1H), 7.54 (d, J=3.0 Hz, 1H), 7.42 (dd, J=3.0, 8.9 Hz, 1H), 6.78-6.86 (m, 2H), 6.67 (br d, J=7.3 Hz, 1H), 4.06 (s, 2H), 3.88 (s, 3H), 3.71 (s, 3H), 3.05-3.10 (m, 2H), 2.75 (t, J=6.5 Hz, 2H), 1.77-1.84 (m, 2H). LC/MS (Table 1, Method B) Rₜ=1.61 min; MS m/z: 352 [M+H]⁺.

### Example #14. 6-methoxy-2-[(5-methoxy-2,3-dihydro-1,4-benzoxazin-4-yl)methyl]-3H-quinazolin-4-one

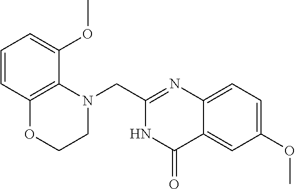 [SMILES: COc1ccc2nc(CN3CCOc4cccc(OC)c43)[nH]c(=O)c2c1]

The compound was synthesized using the same procedure detailed in Example #2 starting from 2-(chloromethyl)-6-methoxy-3H-quinazolin-4-one (CAS RN 1263413-60-0, 100 mg; 0.45 mmol; 1.00 eq.) and 5-methoxy-3,4-dihydro-2H-1,4-benzoxazine (CAS RN 1058704-69-0, 110 mg; 0.67 mmol; 1.50 eq.). The residue was purified by column chromatography (0 to 30% of EtOAc in DCM) giving 6-methoxy-2-[(5-methoxy-2,3-dihydro-1,4-benzoxazin-4-yl)methyl]-3H-quinazolin-4-one (22 mg; 14%) as a white amorphous powder. ¹H NMR (DMSO-d₆) δ: 11.30 (br s, 1H), 7.58 (d, J=8.9 Hz, 1H), 7.53 (d, J=3.0 Hz, 1H), 7.42 (dd, J=3.0, 8.9 Hz, 1H), 6.83 (t, J=8.3 Hz, 1H), 6.55 (dd, J=1.2, 8.3 Hz, 1H), 6.48 (dd, J=1.2, 8.3, 1H), 4.10-4.16 (m, 2H), 4.09 (s, 2H), 3.88 (s, 3H), 3.72 (s, 3H), 3.13-3.18 (m, 2H). LC/MS (Table 1, Method B) Rₜ=1.43 min; MS m/z: 354 [M+H]⁺.

### Example #15. 2-(3,4-dihydro-2H-quinolin-1-ylmethyl)-6-methoxy-3H-quinazolin-4-one

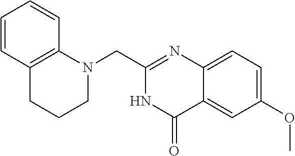 [SMILES: COc1ccc2nc(CN3CCCc4ccccc43)[nH]c(=O)c2c1]

The compound was synthesized using the same procedure detailed in Example #2 starting from 2-(chloromethyl)-6-methoxy-3H-quinazolin-4-one (CAS RN 1263413-60-0, 100 mg; 0.45 mmol; 1.00 eq.) and 1,2,3,4-tetrahydroquinoline (CAS RN 635-46-1, 84 μL; 0.67 mmol; 1.50 eq.). The residue was purified by column chromatography (0 to 2% of MeOH in DCM), following with a recrystallization in ACN giving 2-(3,4-dihydro-2H-quinolin-1-ylmethyl)-6-methoxy-3H-quinazolin-4-one (25 mg; 16%) as a white amorphous powder. ¹H NMR (DMSO-d₆) δ: 12.11 (br s, 1H), 7.53 (d, J=8.9 Hz, 1H1), 7.48 (d, J=3.0 Hz, 1H1), 7.37 (dd, J=3.0, 8.9 Hz, 11), 6.85-6.92 (m, 2H1), 6.46-6.54 (m, 2H), 4.37 (s, 2H), 3.85 (s, 3H), 3.51 (t, J=5.6 Hz, 2H), 2.73 (t, J=6.3 Hz, 2H), 1.92-2.01 (m, 2H). LC/MS (Table 1, Method B) Rₜ=1.53 min; MS m/z: 322 [M+H]⁺.

### Example #16. 2-[(6-methoxy-4-oxo-3H-quinazolin-2-yl)methyl]-3,4-dihydro-1H-isoquinoline-5-carbonitrile

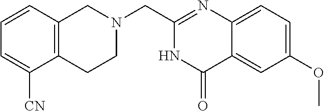 [SMILES: COc1ccc2nc(CN3CCc4c(C#N)cccc4C3)[nH]c(=O)c2c1]

The compound was synthesized using the same procedure detailed in Example #2 starting from 2-(chloromethyl)-6-methoxy-3H-quinazolin-4-one (CAS RN 1263413-60-0, 200 mg; 0.89 mmol; 1.00 eq.) and 1,2,3,4-tetrahydro-5-isoquinolinecarbonitrile (CAS RN 215794-24-4, 211 mg; 1.34 mmol; 1.50 eq.). The residue was purified by column chromatography (5 to 40% of EtOAc in cyclohexane) giving 2-[(6-methoxy-4-oxo-3H-quinazolin-2-yl)methyl]-3,4-dihydro-1H-isoquinoline-5-carbonitrile (251 mg; 81%) as a white amorphous powder. mp=197-218° C. ¹H NMR (DMSO-d₆) δ: 11.96 (s, 1H), 7.65 (dd, J=1.2, 7.6 Hz, 1H), 7.62 (d, J=8.9 Hz, 1H), 7.50 (d, J=3.0 Hz, 1H), 7.43 (br d, J=7.6 Hz, 1H), 7.41 (dd, J=3.0, 8.9 Hz, 1H), 7.32 (t, J=7.7 Hz, 1H), 3.87 (s, 3H), 3.74 (s, 2H), 3.64 (s, 2H), 2.98 (t, J=6.1 Hz, 2H), 2.90 (t, J=6.1 Hz, 2H). LC/MS (Table 1, Method B) Rₜ=1.20 min; MS m/z: 347 [M+H]₊.

### Example #17. 6-methoxy-2-[(5-methoxy-3,4-dihydro-1H-isoquinolin-2-yl)methyl]-3H-quinazolin-4-one

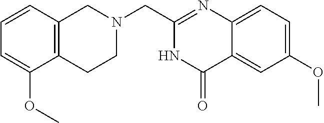 [SMILES: COc1ccc2nc(CN3CCc4c(cccc4OC)C3)[nH]c(=O)c2c1]

The compound was synthesized using the same procedure detailed in Example #2 starting from 2-(chloromethyl)-6-methoxy-3H-quinazolin-4-one (CAS RN 1263413-60-0, 130 mg; 0.58 mmol; 1.00 eq.) and 1,2,3,4-tetrahydro-5-methoxyisoquinoline hydrochloride (CAS RN 103030-69-9, 150 mg; 0.75 mmol; 1.30 eq.) giving 6-methoxy-2-[(5-methoxy-3,4-dihydro-1H-isoquinolin-2-yl)methyl]-3H-quinazolin-4-one (174 mg; 81.5%) as a beige amorphous powder. mp=204-218° C. ¹H NMR (DMSO-d6) δ: 11.94 (br s, 1H), 7.62 (d, J=8.9 Hz, 1H), 7.50 (d, J=3.0 Hz, 1H), 7.41 (dd, J=3.0, 8.9 Hz, 1H), 7.08 (t, J=7.8 Hz, 1H), 6.76 (d, J=7.8 Hz, 1H), 6.64 (d, J=7.8 Hz, 1H), 3.87 (s, 3H), 3.76 (s, 3H), 3.64 (s, 2H), 3.58 (s, 2H), 2.78 (t, J=5.7 Hz, 2H), 2.66 (t, J=5.8 Hz, 2H). LC/MS (Table 1, Method B) Rₜ=1.05 min; MS m/z: 352 [M+H]⁺.

### Example #18. 2-[(5-bromo-3,4-dihydro-1H-isoquinolin-2-yl)methyl]-6-methoxy-3H-quinazolin-4-one

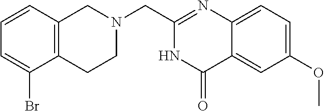 [SMILES: COc1ccc2nc(CN3CCc4c(Br)cccc4C3)[nH]c(=O)c2c1]

The compound was synthesized using the same procedure detailed in Example #1 starting from 2-(chloromethyl)-6-methoxy-3H-quinazolin-4-one (CAS RN 1263413-60-0, 244 mg; 1.09 mmol; 1.00 eq.) and 5-bromo-1,2,3,4-tetrahydroisoquinoline (CAS RN 81237-69-6, 300 mg; 1.41 mmol; 1.30 eq.). The residue was purified by column chromatography (0 to 50% of EtOAc in DCM) giving 2-[(5-bromo-3,4-dihydro-1H-isoquinolin-2-yl)methyl]-6-methoxy-3H-quinazolin-4-one (266 mg; 60%) as a white amorphous powder. ¹H NMR (DMSO-d₆) δ: 11.97 (br s, 1H), 7.62 (d, J=8.9 Hz, 1H), 7.50 (d, J=3.0 Hz, 1H), 7.45 (dd, J=2.0, 7.2 Hz, 1H), 7.41 (dd, J=3.0, 8.9 Hz, 1H), 7.05-7.13 (m, 2H), 3.87 (s, 3H), 3.70 (s, 2H), 3.61 (s, 2H), 2.85 (t, J=6.3 Hz, 2H), 2.77 (t, J=6.3 Hz, 2H). LC/MS (Table 1, Method B) Rₜ=1.39 min; MS m/z: 400 [M+H]⁺.

### Example #19. 6-methoxy-2-[(8-methoxy-3,4-dihydro-1H-isoquinolin-2-yl)methyl]-3H-quinazolin-4-one

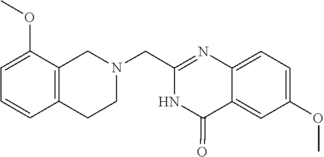

The compound was synthesized using the same procedure detailed in Example #2 starting from 2-(chloromethyl)-6-methoxy-3H-quinazolin-4-one (CAS RN 1263413-60-0, 200 mg; 0.89 mmol; 1.00 eq.) and 8-methoxy-1,2,3,4-tetrahydroisoquinoline (CAS RN 34146-68-4, 218 mg; 1.34 mmol; 1.50 eq.). The residue was purified by column chromatography (5 to 40% of EtOAc in cyclohexane) giving 6-methoxy-2-[(8-methoxy-3,4-dihydro-1H-isoquinolin-2-yl)methyl]-3H-quinazolin-4-one (126 mg; 40%) as a beige amorphous powder. ¹H NMR (400 MHz, DMSO-d₆) δ=11.94 (br s, 1H), 7.63 (d, J=8.9 Hz, 1H), 7.50 (d, J=3.0 Hz, 1H), 7.41 (dd, J=3.0, 8.9 Hz, 1H), 7.11 (t, J=7.8 Hz, 1H), 6.75 (d, J=7.8 Hz, 1H), 6.71 (d, J=7.8 Hz, 1H), 3.87 (s, 3H), 3.73 (s, 3H), 3.62 (s, 2H), 3.56 (s, 2H), 2.85-2.79 (m, 2H), 2.79-2.73 (m, 2H). LC/MS (Table 1, Method B) Rₜ=1.05 min; MS m/z: 352 [M+H]*.

### Example #20. 2-[(5-chloro-3,4-dihydro-1H-isoquinolin-2-yl)methyl]-6-methoxy-3H-quinazolin-4-one

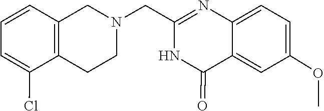 [SMILES: COc1ccc2nc(CN3CCc4c(Cl)cccc4C3)[nH]c(=O)c2c1]

The compound was synthesized using the same procedure detailed in Example #1 starting from 2-(chloromethyl)-6-methoxy-3H-quinazolin-4-one (CAS RN 1263413-60-0, 130 mg; 0.58 mmol; 1.00 eq.) and 5-chloro-1,2,3,4-tetrahydroisoquinoline (CAS RN 73075-43-1, 126 mg; 0.75 mmol; 1.30 eq.). The residue was purified by column chromatography (0 to 50% of EtOAc in DCM) giving 2-[(5-chloro-3,4-dihydro-1H-isoquinolin-2-yl)methyl]-6-methoxy-3H-quinazolin-4-one (128 mg; 62%) as a white amorphous powder. ¹H NMR (400 MHz, DMSO-d₆) δ=11.98 (s, 1H), 7.62 (d, J=8.9 Hz, 1H), 7.50 (d, J=3.0 Hz, 1H), 7.41 (dd, J=3.0, 8.9 Hz, 1H), 7.28 (dd, J=1.0, 7.8 Hz, 1H), 7.15 (t, J=7.8 Hz, 1H), 7.07 (dd, J=1.0, 7.8 Hz, 1H), 3.87 (s, 3H), 3.70 (s, 2H), 3.61 (s, 2H), 2.89-2.83 (m, 2H), 2.83-2.77 (m, 2H). LC/MS (Table 1, Method B) Rₜ=1.34 min; MS m/z: 356 [M+H]₊.

### Example #21. N-[2-[(6-methoxy-4-oxo-3H-quinazolin-2-yl)methyl]-3,4-dihydro-1H-isoquinolin-5-yl]methanesulfonamide

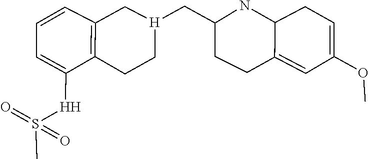

### Step A: 2-[(5-amino-3,4-dihydro-1H-isoquinolin-2-yl)methyl]-6-methoxy-3H-quinazolin-4-one

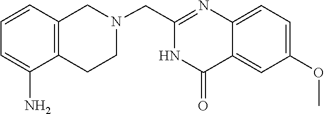 [SMILES: COc1ccc2nc(CN3CCc4c(N)cccc4C3)[nH]c(=O)c2c1]

The compound was synthesized using the same procedure detailed in Example #1 starting from 2-(chloromethyl)-6-methoxy-3H-quinazolin-4-one (CAS RN 1263413-60-0, 100 mg; 0.45 mmol; 1.00 eq.) and 1,2,3,4-tetrahydro-5-aminoisoquinoline (CAS RN 115955-90-3, 66 mg; 0.45 mmol; 1.00 eq.). The residue was purified by column chromatography (5% of MeOH in DCM) giving 2-[(5-amino-3,4-dihydro-1H-isoquinolin-2-yl)methyl]-6-methoxy-3H-quinazolin-4-one (60 mg; 40%) as a white amorphous powder. mp=211-233° C. ¹H NMR (400 MHz, DMSO-d₆) δ=11.90 (br s, 1H), 7.62 (d, J=8.9 Hz, 1H), 7.50 (d, J=3.0 Hz, 1H), 7.41 (dd, J=3.0, 8.9 Hz, 1H), 6.80 (t, J=7.6 Hz, 1H), 6.44 (d, J=7.6 Hz, 1H), 6.23 (d, J=7.6 Hz, 1H), 4.77 (s, 2H), 3.87 (s, 3H), 3.56 (s, 4H), 2.80 (t, J=5.7 Hz, 2H), 2.48 (t, J=5.7 Hz, 2H). LC/MS (Table 1, Method B) Rₜ=0.80 min; MS m/z: 337 [M+H]⁺.

### Step B: N-[2-[(6-methoxy-4-oxo-3H-quinazolin-2-yl)methyl]-3,4-dihydro-1H-isoquinolin-5-yl]methanesulfonamide

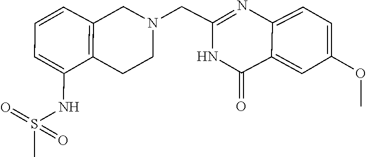 [SMILES: COc1ccc2nc(CN3CCc4c(cccc4NS(C)(=O)=O)C3)[nH]c(=O)c2c1]

2-[(5-amino-3,4-dihydro-1H-isoquinolin-2-yl)methyl]-6-methoxy-3H-quinazolin-4-one, Example #21, step A, (20 mg; 0.06 mmol; 1.00 eq.) was partially dissolved in DCM (0.40 mL) at rt under N₂ atm, then TEA (11 μL; 0.08 mmol; 1.30 eq.) and MsCl (5 μL; 0.07 mmol; 1.10 eq.) were added. The reaction mixture was stirred at rt. Reaction media was concentrated to dryness. The residue was purified by preparative LC-MS (Table 2, Conditions 1)(column: Kinetex C 18, 30×150 mm 5 μm (Phenomenex); flow rate: 42 mL/min; Mobile phase: H₂O with 0.1% of HCOOH/ACN with 0.1% of HCOOH; gradient: 25 to 40% of ACN with 0.1% of HCOOH; run 20 min) to give N-[2-[(6-methoxy-4-oxo-3H-quinazolin-2-yl)methyl]-3,4-dihydro-JH-isoquinolin-5-yl]methanesulfonamide (10 mg; 38%) as a white amorphous powder. ¹H NMR (400 MHz, DMSO-d₆) δ=11.88 (br s, 1H), 8.97 (br s, 1H), 7.62 (d, J=8.9 Hz, 1H), 7.50 (d, J=3.0 Hz, 1H), 7.41 (dd, J=3.0, 8.9 Hz, 1H), 7.18-7.10 (m, 2H), 6.96 (dd, J=1.8, 7.0 Hz, 1H), 3.87 (s, 3H), 3.68 (s, 2H), 3.59 (s, 2H), 2.98 (s, 3H), 2.90-2.84 (m, 2H), 2.82-2.77 (m, 2H). LC/MS (Table 1, Method B) Rₜ=0.91 min; MS m/z: 415 [M+H]⁺.

### Example #22. 6-methoxy-2-(2,3,4,5-tetrahydro-1-benzazepin-1-ylmethyl)-3H-quinazolin-4-one

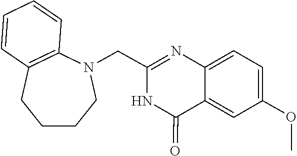 [SMILES: COc1ccc2nc(CN3CCCCc4ccccc43)[nH]c(=O)c2c1]

The compound was synthesized using the same procedure detailed in Example #2 starting from 2-(chloromethyl)-6-methoxy-3H-quinazolin-4-one (CAS RN 1263413-60-0, 100 mg; 0.45 mmol; 1.00 eq.) and 1,2,3,4-tetrahydro-1H-1-benzazepine (CAS RN 1701-57-1, 197 mg; 1.34 mmol; 1.50 eq.). The residue was purified by column chromatography (5 to 40% of EtOAc in cyclohexane) giving 6-methoxy-2-(2,3,4,5-tetrahydro-1-benzazepin-1-ylmethyl)-3H-quinazolin-4-one (78 mg; 26%) as a white amorphous powder. mp=191-206° C. ¹H NMR (400 MHz, DMSO-d₆) δ=11.99 (br s, 1H), 7.61 (d, J=8.9 Hz, 1H), 7.49 (d, J=3.0 Hz, 1H), 7.40 (dd, J=3.0, 8.9 Hz, 1H), 7.12-7.06 (m, 2H), 6.98 (dd, J=0.9, 8.0 Hz, 1H), 6.84 (dt, J=1.2, 7.4 Hz, 1H), 4.23 (s, 2H), 3.86 (s, 3H), 3.01-2.93 (m, 2H), 2.89-2.80 (m, 2H), 1.67-1.58 (m, 2H), 1.57-1.48 (m, 2H). LC/MS (Table 1, Method B) Rₜ=1.65 min; MS m/z: 336 [M+H]⁺.

### Example #23. 6-methoxy-2-[(1-methyl-3,4-dihydro-2H-1,5-benzodiazepin-5-yl)methyl]-3H-quinazolin-4-one

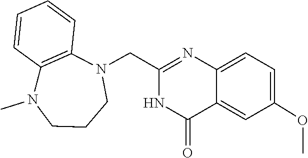

The compound was synthesized using the same procedure detailed in Example #2 starting from 2-(chloromethyl)-6-methoxy-3H-quinazolin-4-one (CAS RN 1263413-60-0, 400 mg; 1.78 mmol; 1.00 eq.) and 1-methyl-2,3,4,5-tetrahydro-1,5-benzodiazepine (CAS RN 32900-36-0, 433 mg; 2.67 mmol; 1.50 eq.). The residue was purified by column chromatography (0 to 20% of EtOAc in cyclohexane) giving 6-methoxy-2-[(1-methyl-3,4-dihydro-2H-1,5-benzodiazepin-5-yl)methyl]-3H-quinazolin-4-one (170 mg; 27%) as a brown amorphous powder. ¹H NMR (400 MHz, DMSO-d₆) δ=11.91 (s, 1H), 7.56 (d, J=8.9 Hz, 1H), 7.47 (d, J=3.0 Hz, 1H), 7.38 (dd, J=3.0, 8.9 Hz, 1H), 6.93 (dd, J=1.3, 7.8 Hz, 1H), 6.89-6.82 (m, 2H), 6.81-6.75 (m, 1H), 4.30 (s, 2H), 3.85 (s, 3H), 3.22 (t, J=5.8 Hz, 2H), 3.06 (t, J=5.8 Hz, 2H), 2.83 (s, 3H), 1.73 (quin, J=5.8 Hz, 2H). LC/MS (Table 1, Method B) Rₜ=1.04 min; MS m/z: 351 [M+H]⁺.

### Example #36. 6-chloro-2-(indolin-1-ylmethyl)-3H-quinazolin-4-one (CAS RN 1088219-04-8)

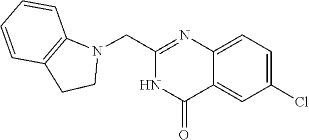 [SMILES: O=c1[nH]c(CN2CCc3ccccc32)nc2ccc(Cl)cc12]

### Example #37. 6-chloro-2-[(3-methylindolin-1-yl)methyl]-3H-quinazolin-4-one

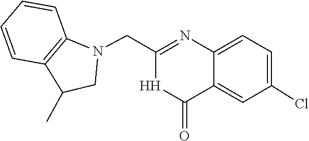

The compound was synthesized using the same procedure detailed in Example #2 starting from 2-(chloromethyl)-6-chloro-3H-quinazolin-4-one (CAS RN 2856-54-4, 400 mg; 1.75 mmol; 1.00 eq.) and 3-methyl-2,3-dihydro-1H-indole hydrochloride (CAS RN 4375-15-9, 385 mg; 2.27 mmol; 1.30 eq.). The residue was purified by column chromatography (0 to 30% of EtOAc in cyclohexane) giving 6-chloro-2-[(3-methylindolin-1-yl)methyl]quinazolin-4-one (394 mg; 69%) as a white amorphous powder. mp=200-223° C. ¹H NMR (400 MHz, DMSO-d₆) δ=12.50 (br s, 1H), 8.05 (d, J=2.5 Hz, 1H), 7.83 (dd, J=2.5, 8.7 Hz, 1H), 7.67 (d, J=8.7 Hz, 1H), 7.06 (br d, J=7.2 Hz, 1H), 6.99 (br t, J=7.7 Hz, 1H), 6.65 (dt, J=0.8, 7.2 Hz, 1H), 6.59 (d, J=7.7 Hz, 1H), 4.29 (d, J=15.4 Hz, 1H), 4.09 (d, J=15.4 Hz, 1H), 3.72 (t, J=8.6 Hz, 1H), 3.31-3.21 (m, 1H), 3.00 (t, J=8.6 Hz, 1H), 1.26 (d, J=6.7 Hz, 3H). LC/MS (Table 1, Method B) Rₜ=1.71 min; MS m/z: 326 [M+H]⁺.

### Example #38. 6-chloro-2-[(3-methylindolin-1-yl)methyl]-3H-quinazolin-4-one Enantiomer 1

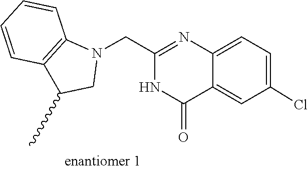 [SMILES: CC1CN(Cc2nc3ccc(Cl)cc3c(=O)[nH]2)c2ccccc21]

Example #37 was separated by chiral LC-MS (Table 2, Condition 5) (column: Chiralpack IA, 250×20 mm, 5 μm, Mobile phase: [heptane 97%—EtOH 3%], Flow rate: 18 mL/min, T° column: 25° C.) giving 6-chloro-2-[(3-methylindolin-1-yl)methyl]-3H-quinazolin-4-one enantiomer 1 (ee>98.7%) (90 mg; 16%) as a white amorphous powder. ¹H NMR (400 MHz, DMSO-d₆) δ=12.48 (br s, 1H), 8.04 (d, J=2.5 Hz, 1H), 7.81 (dd, J=2.5, 8.7 Hz, 1H), 7.65 (d, J=8.7 Hz, 1H), 7.05 (d, J=7.4 Hz, 1H), 6.99 (t, J=7.6 Hz, 1H), 6.64 (dt, J=0.9, 7.4 Hz, 1H), 6.59 (d, J=7.6 Hz, 1H), 4.28 (d, J=15.4 Hz, 1H), 4.08 (d, J=15.4 Hz, 1H), 3.73 (t, J=8.6 Hz, 1H), 3.30-3.21 (m, 1H), 3.00 (t, J=8.6 Hz, 1H), 1.26 (d, J=6.8 Hz, 3H). LC/MS (Table 1, Method B) Rₜ=1.72 min; MS m/z: 326 [M+H]⁺. LC/MS (Table 1, Method F) Rₜ=22.1 min.

### Example #39. 6-chloro-2-[(3-methylindolin-1-yl)methyl]-3H-quinazolin-4-one Enantiomer 2

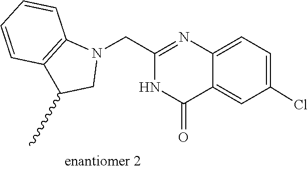 [SMILES: CC1CN(Cc2nc3ccc(Cl)cc3c(=O)[nH]2)c2ccccc21]

Example #37 was separated by chiral LC-MS (Table 2, Condition 5) (column: Chiralpack IA, 250×20 mm, 5 μm, Mobile phase: [heptane 97%—EtOH 3%], Flow rate: 18 mL/min, T° column: 25° C.) giving 6-chloro-2-[(3-methylindolin-1-yl)methyl]-3H-quinazolin-4-one enantiomer 2 (ee>98.5%) (12 mg; 2%) as a white amorphous powder. ¹H NMR (400 MHz, DMSO-d₆) δ=12.52 (br s, 1H), 8.04 (d, J=2.5 Hz, 1H), 7.81 (dd, J=2.5, 8.7 Hz, 1H), 7.66 (d, J=8.7 Hz, 1H), 7.05 (d, J=7.4 Hz, 1H), 6.99 (t, J=7.6 Hz, 1H), 6.64 (dt, J=0.9, 7.4 Hz, 1H), 6.59 (d, J=7.6 Hz, 1H), 4.28 (d, J=15.4 Hz, 1H), 4.08 (d, J=15.4 Hz, 1H), 3.73 (t, J=8.6 Hz, 1H), 3.30-3.20 (m, 1H), 3.00 (t, J=8.6 Hz, 1H), 1.26 (d, J=6.8 Hz, 3H). LC/MS (Table 1, Method B) Rₜ=1.72 min; MS m/z: 326 [M+H]⁺. LC/MS (Table 1, Method F) Rₜ=27.9 min.

### Example #40. 6-chloro-2-[(3,3-dimethylindolin-1-yl)methyl]-3H-quinazolin-4-one

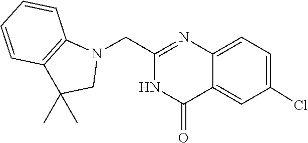 [SMILES: CC1(C)CN(Cc2nc3ccc(Cl)cc3c(=O)[nH]2)c2ccccc21]

The compound was synthesized using the same procedure detailed in Example #2 starting from 2-(chloromethyl)-6-chloro-3H-quinazolin-4-one (CAS RN 2856-54-4, 200 mg; 0.87 mmol; 1.00 eq.) and 3,3-dimethylindoline (CAS RN 1914-02-9, 193 mg; 1.31 mmol; 1.50 eq.). The residue was purified by column chromatography (5 to 40% of EtOAc in cyclohexane) giving 6-chloro-2-[(3,3-dimethylindolin-1-yl)methyl]-3H-quinazolin-4-one (178 mg; 60%) as a yellow amorphous powder. mp=163-176° C. ¹H NMR (400 MHz, DMSO-d₆) δ=12.50 (s, 1H), 8.05 (d, J=2.5 Hz, 1H), 7.83 (dd, J=2.5, 8.7 Hz, 1H), 7.66 (d, J=8.7 Hz, 1H), 7.03 (dd, J=0.9, 7.3 Hz, 1H), 6.99 (dt, J=1.3, 7.7 Hz, 1H), 6.65 (dt, J=0.9, 7.3 Hz, 1H), 6.60 (d, J=7.7 Hz, 1H), 4.21 (s, 2H), 3.28 (s, 2H), 1.26 (s, 6H). LC/MS (Table 1, Method B) Rₜ=1.82 min; MS m/z: 340 [M+H]⁺.

### Example #41. 6-chloro-2-[(2-methylindolin-1-yl)methyl]-3H-quinazolin-4-one

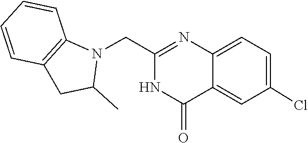 [SMILES: CC1Cc2ccccc2N1Cc1nc2ccc(Cl)cc2c(=O)[nH]1]

The Example #41 was synthesized using the same procedure as example #52. 1H NMR (DMSO-d6) δ: 12.34 (1H, s), 8.04 (1H, d), 7.82 (1H, dd), 7.64 (1H, d), 7.03 (1H, d), 6.94 (1H, ddd,), 6.59 (1H, ddd), 6.44 (1H, d), 4.29 (1H, d), 4.23 (1H, d), 4.03-3.91 (1H, m), 3.16 (1H, dd), 2.60 (1H, dd), 1.25 (3H, d). MS (ES)+: 326/328 (M+H)+

### Example #42. 6-chloro-2-(spiro[cyclopentane-1,3′-indoline]-1′-ylmethyl)-3H-quinazolin-4-one

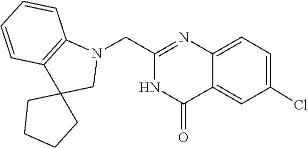 [SMILES: O=c1[nH]c(CN2CC3(CCCC3)c3ccccc32)nc2ccc(Cl)cc12]

The compound was synthesized using the same procedure detailed in Example #2 starting from 2-(chloromethyl)-6-chloro-3H-quinazolin-4-one (CAS RN 2856-54-4, 200 mg; 0.87 mmol; 1.00 eq.) and 1′,2′-dihydrospiro[cyclopentane-1,3′-indole] (CAS RN 7131-05-7, 227 mg; 1.31 mmol; 1.50 eq.). The residue was purified by preparative chromatography (Table 2, Conditions 1) (column: Kinetex C18.30×150 mm 5 μm (phenomenex); Flow rate: 42 mL/min; Mobile phase: H₂O+0.1% HCOOH/ACN+0.1% HCOOH; Gradient: 70-85%; run 12 min) giving 6-chloro-2-(spiro[cyclopentane-1,3′-indoline]-1′-ylmethyl)-3H-quinazolin-4-one (102 mg; 32%) as a beige amorphous powder. ¹H NMR (400 MHz, DMSO-d₆) δ=12.51 (br s, 1H), 8.05 (d, J=2.5 Hz, 1H), 7.83 (dd, J=2.5, 8.7 Hz, 1H), 7.67 (d, J=8.7 Hz, 1H), 7.04 (dd, J=0.9, 7.3 Hz, 1H), 6.98 (dt, J=1.2, 7.7 Hz, 1H), 6.65 (dt, J=0.9, 7.3 Hz, 1H), 6.59 (d, J=7.7 Hz, 1H), 4.19 (s, 2H), 3.32 (s, 2H), 1.88-1.61 (m, 8H). LC/MS (Table 1, Method B) Rₜ=1.95 min; MS m/z: 366 [M+H]⁺.

### Example #43. 6-chloro-2-[(4-methylindolin-1-yl)methyl]-3H-quinazolin-4-one

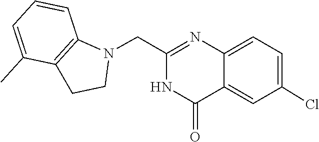 [SMILES: Cc1cccc2c1CCN2Cc1nc2ccc(Cl)cc2c(=O)[nH]1]

The Example #43 was synthesized using the same procedure as example #47.

1H NMR (500 MHz, DMSO-d6) δ 12.44 (br s, 1H), 8.04 (d, 1H), 7.82 (dd, 1H), 7.67 (d, 1H), 6.88 (t, 1H), 6.45 (d, 1H), 6.40 (d, 1H), 4.18 (s, 2H), 3.53-3.48 (m, 2H), 2.89-2.83 (m, 2H), 2.14 (s, 3H). MS (ES+): 326/328 (M+H)+

### Example #44. 6-chloro-2-[(7-methylindolin-1-yl)methyl]-3H-quinazolin-4-one

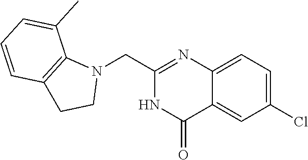 [SMILES: Cc1cccc2c1N(Cc1nc3ccc(Cl)cc3c(=O)[nH]1)CC2]

The Example #44 was synthesized using the same procedure as example #47.

1H NMR (500 MHz, DMSO-d6) δ 12.38 (s, 1H), 8.04 (d, 1H), 7.80 (dd, 1H), 7.60 (d, 1H), 6.92 (d, 1H), 6.77 (d, 1H), 6.60 (t, 1H), 4.41 (s, 2H), 3.47 (t, 2H), 2.94 (t, 2H), 2.26 (s, 3H). MS (ES+): 326/328 (M+H)+

### Example #45. 6-chloro-2-[(6-methylindolin-1-yl)methyl]-3H-quinazolin-4-one

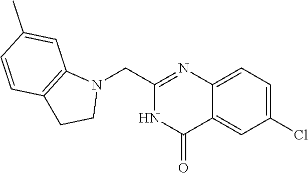 [SMILES: Cc1ccc2c(c1)N(Cc1nc3ccc(Cl)cc3c(=O)[nH]1)CC2]

The Example #45 was synthesized using the same procedure as example #47.

1H NMR (500 MHz, DMSO-d6) δ 12.44 (s, 1H), 8.05 (d, 1H), 7.83 (dd, 1H), 7.67 (d, 1H), 6.93 (d, 1H), 6.48-6.37 (m, 2H), 4.18 (s, 2H), 3.47 (t, 2H), 2.87 (t, 2H), 2.18 (s, 3H). MS (ES+): 326/328 (M+H)+

### Example #46. 6-chloro-2-[(5-methylindolin-1-yl)methyl]-3H-quinazolin-4-one

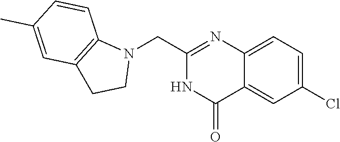 [SMILES: Cc1ccc2c(c1)CCN2Cc1nc2ccc(Cl)cc2c(=O)[nH]1]

The Example #46 was synthesized using the same procedure as example #47.

1H NMR (500 MHz, DMSO-d6) δ 12.43 (s, 1H), 8.04 (d, 1H), 7.82 (dd, 1H), 7.67 (d, 1H), 6.89 (s, 1H), 6.84-6.76 (m, 1H), 6.49 (d, J=7.9 Hz, 1H), 4.15 (s, 2H), 3.44 (t, 2H), 2.88 (t, 2H), 2.17 (s, 3H). MS (ES+): 326/328 (M+H)+

### Example #47. 6-chloro-2-[(5-fluoroindolin-1-yl)methyl]-3H-quinazolin-4-one

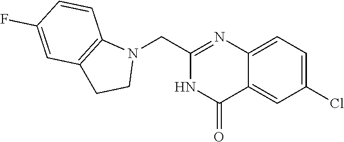 [SMILES: O=c1[nH]c(CN2CCc3cc(F)ccc32)nc2ccc(Cl)cc12]

A suspension of 6-chloro-2-(chloromethyl)quinazolin-4(3H)-one (50 mg, 0.218 mmol) and triethylamine (0.091 ml, 0.655 mmol) in EtOH (2 mL) was treated with 5-fluoroindoline (35.9 mg, 0.262 mmol) and the resulting suspension was stirred at 80° C. for 22 h. The reaction mixture was concentrated onto silica and the crude product was purified by chromatography on silica gel (12 g cartridge, 0-100% EtOAc(+0.5% Et3N)/isohexane) to afford 6-chloro-2-((5-fluoroindolin-1-yl)methyl)quinazolin-4(3H)-one (36.6 mg, 50.3%) as a flocculent white solid. 1H NMR (500 MHz, DMSO-d6) δ 12.46 (br s, 1H), 8.04 (d, 1H), 7.83 (dd, 1H), 7.66 (d, 1H), 6.94 (dd, 1H), 6.80 (ddd, 1H), 6.55 (dd, 1H), 4.17 (s, 2H), 3.50 (app. t, 2H), 2.93 (app. t, 2H). MS (ES+): 330/332 (M+H)+.

### Example #48. 2-[(5-bromoindolin-1-yl)methyl]-6-chloro-3H-quinazolin-4-one

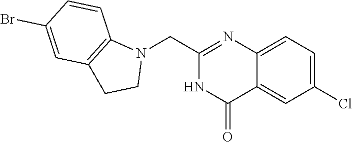 [SMILES: O=c1[nH]c(CN2CCc3cc(Br)ccc32)nc2ccc(Cl)cc12]

The Example #48 was synthesized using the same procedure as example #47. 1H NMR (500 MHz, DMSO-d6) δ 12.48 (s, 1H), 8.04 (d, 1H), 7.82 (dd, 1H), 7.65 (d, 1H), 7.19 (d, 1H), 7.12 (dd, 1H), 6.53 (d, 1H), 4.22 (s, 2H), 3.54 (t, 2H), 2.96 (t, 2H). MS (ES+): 390/392 (M+H)+

### Example #49. 6-chloro-2-[(7-methoxyindolin-1-yl)methyl]-3H-quinazolin-4-one

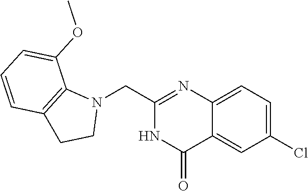 [SMILES: COc1cccc2c1N(Cc1nc3ccc(Cl)cc3c(=O)[nH]1)CC2]

The Example #49 was synthesized using the same procedure as example #52. 1H NMR (DMSO-d6) δ: 12.45 (1H, s), 8.05 (1H, d), 7.83 (1H, dd), 7.67 (1H, d), 6.97 (1H, dd), 6.33 (1H, d), 6.27 (1H, d), 4.20 (2H, s), 3.74 (3H, s), 3.52 (2H, t), 2.85 (2H, t). MS (ES+): 342/344 (M+H)+

### Example #50. 6-chloro-2-(isoindolin-2-ylmethyl)-3H-quinazolin-4-one

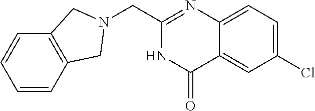 [SMILES: O=c1[nH]c(CN2Cc3ccccc3C2)nc2ccc(Cl)cc12]

The compound was synthesized using the same procedure detailed in Example #1 starting from 2-(chloromethyl)-6-chloro-3H-quinazolin-4-one (CAS RN 2856-54-4, 200 mg; 0.87 mmol; 1.00 eq.) and isoindoline (CAS RN 496-12-8, 198 μL; 1.75 mmol; 2.00 eq.). The residue was purified by column chromatography (0 to 40% of EtOAc in cyclohexane) giving 6-chloro-2-(isoindolin-2-ylmethyl)-3H-quinazolin-4-one (76 mg; 28%) as a white amorphous powder. ¹H NMR (500 MHz, DMSO-d₆) δ=12.30 (br s, 1H), 8.05 (d, J=2.5 Hz, 1H), 7.84 (dd, J=2.5, 8.7 Hz, 1H), 7.70 (d, J=8.7 Hz, 1H), 7.27-7.22 (m, 2H), 7.22-7.17 (m, 2H), 4.01 (s, 4H), 3.85 (s, 2H). LC/MS (Table 1, Method B) Rₜ=0.99 min; MS m/z: 312 [M+H]⁺.

### Example #51. 6-chloro-2-(3,4-dihydro-2H-quinolin-1-ylmethyl)-3H-quinazolin-4-one (CAS RN 1325134-44-8)

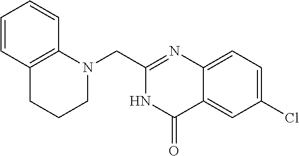 [SMILES: O=c1[nH]c(CN2CCCc3ccccc32)nc2ccc(Cl)cc12]

### Example #52. 6-chloro-2-[(5-methoxy-3,4-dihydro-2H-quinolin-1-yl)methyl]-3H-quinazolin-4-one

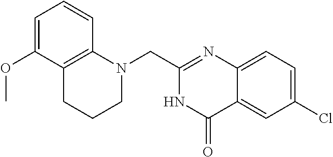 [SMILES: COc1cccc2c1CCCN2Cc1nc2ccc(Cl)cc2c(=O)[nH]1]

A mixture of 6-chloro-2-(chloromethyl)quinazolin-4(3H)-one (Preparation #10, 0.1 g, 0.437 mmol), 5-methoxy-1,2,3,4-tetrahydroquinoline (0.086 g, 0.524 mmol), and potassium carbonate (0.121 g, 0.873 mmol) in EtOH (1.5 mL) was stirred at 70° C. overnight, then allowed to cool to room temperature. Sat. NH4Cl (aq.) and DCM were added and the phases were separated through a phase separator. The organic extract was washed with brine and absorbed on silica. The crude product was purified by chromatography on silica gel (4 g cartridge, 0-50% EtOAc/isohexane) to afford 6-chloro-2-((5-methoxy-3,4-dihydroquinolin-1(2H)-yl)methyl)quinazolin-4(3H)-one (0.060 g, 38.2%) as a tan solid. 1H NMR (DMSO-d6) δ: 11.43 (1H, s), 8.08 (1H, d), 7.84 (1H, dd), 7.66 (1H, d), 6.88-6.73 (2H, m), 6.67 (1H, d), 4.11 (2H, s), 3.67 (3H, s), 3.18-3.04 (2H, m), 2.74 (2H, dd), 1.89-1.68 (2H, m). MS (ES+): 356/358 (M+H)+

### Example #53. 6-chloro-2-[(5-methyl-3,4-dihydro-2H-quinolin-1-yl)methyl]-3H-quinazolin-4-one

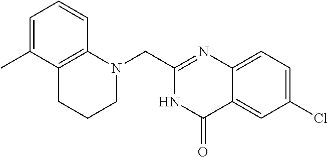 [SMILES: Cc1cccc2c1CCCN2Cc1nc2ccc(Cl)cc2c(=O)[nH]1]

The compound was synthesized using the same procedure detailed in Example #1 starting from 2-(chloromethyl)-6-chloro-3H-quinazolin-4-one (CAS RN 2856-54-4,210 mg; 0.92 mmol; 1.00 eq.) and 5-methyl-1,2,3,4-tetrahydroquinoline (CAS RN 58960-02-4, 161 mg, 1.01 mmol, 1.10 eq.). The residue was purified by column chromatography (0 to 25% of EtOAc in cyclohexane) giving 6-chloro-2-[(5-methyl-3,4-dihydro-2H-quinolin-1-yl)methyl]-3H-quinazolin-4-one (143 mg; 45%) as an off-white amorphous powder. ¹H NMR (400 MHz, DMSO-d6) δ=12.33 (br s, 1H), 8.02 (d, J=2.5 Hz, 1H), 7.79 (dd, J=2.5, 8.7 Hz, 1H), 7.60 (d, J=8.7 Hz, 1H), 6.78 (t, J=7.8 Hz, 1H), 6.42 (d, J=7.6 Hz, 1H), 6.37 (d, J=8.1 Hz, 1H), 4.38 (s, 2H), 3.47 (t, J=5.2 Hz, 2H), 2.61 (t, J=6.5 Hz, 2H), 2.12 (s, 3H), 2.04-1.96 (m, 2H). LC/MS (Table 1, Method B) Rₜ=1.80 min; MS m/z: 340 [M+H]⁺.

### Example #54. 6-chloro-2-[(8-methoxy-3,4-dihydro-2H-quinolin-1-yl)methyl]-3H-quinazolin-4-one

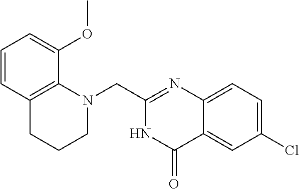 [SMILES: COc1cccc2c1N(Cc1nc3ccc(Cl)cc3c(=O)[nH]1)CCC2]

The compound was synthesized using the same procedure detailed in Example #2 starting from 2-(chloromethyl)-6-chloro-3H-quinazolin-4-one (CAS RN2856-54-4, 100 mg; 0.44 mmol; 1.00 eq.) and 8-methoxy-1,2,3,4-tetrahydroquinoline (CAS RN 53899-17-5, 107 mg; 0.66 mmol; 1.50 eq.). The residue was purified by preparative chromatography (Table 2, conditions 1) (column: Kinetex C18.30×150 mm 5 μm (phenomenex); Flow rate: 42 mL/min; Mobile phase: H₂O+0.1% HCOOH/ACN+0.1% HCOOH; Gradient: 50-65%; run 12 min) and then purified by column chromatography (0 to 20% of EtOAc in cyclohexane) giving 6-chloro-2-[(8-methoxy-3,4-dihydro-2H-quinolin-1-yl)methyl]-3H-quinazolin-4-one (49 mg; 31%) as a white amorphous powder. mp=183-194° C. ¹H NMR (500 MHz, DMSO-d₆) δ=11.43 (br s, 1H), 8.08 (d, J=2.5 Hz, 1H), 7.84 (dd, J=2.5, 8.7 Hz, 1H), 7.66 (d, J=8.7 Hz, 1H), 6.85-6.79 (m, 1H), 6.77 (d, J=7.0 Hz, 1H), 6.67 (d, J=7.0 Hz, 1H), 4.11 (s, 2H), 3.67 (s, 3H), 3.15-3.08 (m, 2H), 2.74 (t, J=6.4 Hz, 2H), 1.85-1.77 (m, 2H). LC/MS (Table 1, Method B) R^{t}=1.80 min; MS m/z: 356 [M+H]⁺.

### Example #55. 6-chloro-2-[(7-methoxy-3,4-dihydro-2H-quinolin-1-yl)methyl]-3H-quinazolin-4-one

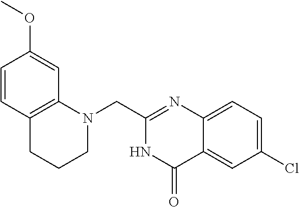 [SMILES: COc1ccc2c(c1)N(Cc1nc3ccc(Cl)cc3c(=O)[nH]1)CCC2]

The compound was synthesized using the same procedure detailed in Example #1 starting from 2-(chloromethyl)-6-chloro-3H-quinazolin-4-one (CAS RN 2856-54-4, 150 mg; 0.65 mmol; 1.00 eq.) and 7-methoxy-1,2,3,4-tetrahydroquinoline (CAS RN 19500-61-9, 0.11 mL; 0.72 mmol; 1.10 eq.).

The residue was purified by column chromatography (0 to 50% of EtOAc in cyclohexane) giving 6-chloro-2-[(7-methoxy-3,4-dihydro-2H-quinolin-1-yl)methyl]-3H-quinazolin-4-one (60 mg; 24%) as a beige amorphous powder. ¹H NMR (500 MHz, DMSO-d₆) δ=12.42 (br s, 1H), 8.04 (d, J=2.2 Hz, 1H), 7.80 (dd, J=2.2, 8.7 Hz, 1H), 7.61 (d, J=8.7 Hz, 1H), 6.80 (br d, J=8.5 Hz, 1H), 6.14-6.08 (m, 2H), 4.38 (s, 2H), 3.57 (s, 3H), 3.51 (br t, J=5.4 Hz, 2H), 2.66 (br t, J=6.2 Hz, 2H), 1.97-1.89 (m, 2H). LC/MS (Table 1, Method B) Rₜ=1.70 min; MS m/z: 356 [M+H]+.

### Example #57. 6-chloro-2-(2,3-dihydro-1,4-benzoxazin-4-ylmethyl)-3H-quinazolin-4-one

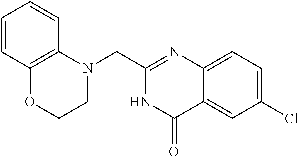 [SMILES: O=c1[nH]c(CN2CCOc3ccccc32)nc2ccc(Cl)cc12]

The compound was synthesized using the same procedure detailed in Example #2 starting from 2-(chloromethyl)-6-chloro-3H-quinazolin-4-one (CAS RN 2856-54-4, 200 mg; 0.87 mmol; 1.00 eq.) and 3,4-dihydro-2H-1,4-benzoxazine (CAS RN 5735-53-5, 162 μL; 1.31 mmol; 1.50 eq.). The residue was purified by column chromatography (5 to 40% of EtOAc in cyclohexane) giving 6-chloro-2-(2,3-dihydro-1,4-benzoxazin-4-ylmethyl)-3H-quinazolin-4-one (53 mg; 18%) as a white amorphous powder. ¹H NMR (500 MHz, DMSO-d₆) δ=12.45 (br s, 1H), 8.04 (d, J=2.5 Hz, 1H), 7.81 (dd, J=2.5, 8.7 Hz, 1H), 7.63 (d, J=8.7 Hz, 1H), 6.71-6.66 (m, 2H), 6.66-6.62 (m, 1H), 6.55 (dt, J=1.7, 7.4 Hz, 1H), 4.42 (s, 2H), 4.32-4.27 (m, 2H), 3.60-3.54 (m, 2H). LC/MS (Table 1, Method B) Rₜ=1.53 min; MS m/z: 328 [M+H]*.

### Example #58. 6-chloro-2-[(5-methoxy-3,4-dihydro-1H-isoquinolin-2-yl)methyl]-3H-quinazolin-4-one

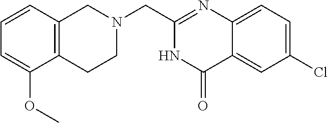 [SMILES: COc1cccc2c1CCN(Cc1nc3ccc(Cl)cc3c(=O)[nH]1)C2]

The compound was synthesized using the same procedure detailed in Example #2 starting from 2-(chloromethyl)-6-chloro-3H-quinazolin-4-one (CAS RN 2856-54-4, 70 mg; 0.31 mmol; 1.00 eq.) and 1,2,3,4-tetrahydro-5-methoxyisoquinoline hydrochloride (CAS RN 103030-70-2, 262 mg; 1.31 mmol; 1.50 eq.). The residue was purified by column chromatography (5 to 40% of EtOAc in cyclohexane) giving 6-chloro-2-[(5-methoxy-3,4-dihydro-1H-isoquinolin-2-yl)methyl]-3H-quinazolin-4-one (52 mg; 16.5%) as a white amorphous powder. mp=205-212° C. ¹H NMR (500 MHz, DMSO-d₆) δ=12.17 (br s, 1H), 8.04 (d, J=2.5 Hz, 1H), 7.84 (dd, J=2.5, 8.7 Hz, 1H), 7.70 (d, J=8.7 Hz, 1H), 7.08 (t, J=8.0 Hz, 1H), 6.76 (d, J=8.0 Hz, 1H), 6.64 (d, J=7.7 Hz, 1H), 3.76 (s, 3H), 3.66 (s, 2H), 3.61 (s, 2H), 2.80 (t, J=6.0 Hz, 2H), 2.66 (t, J=6.0 Hz, 2H). LC/MS (Table 1, Method B) Rₜ=1.23 min; MS m/z: 356 [M+H]⁺.

### Example #59. 6-chloro-2-(3,4-dihydro-1H-isoquinolin-2-ylmethyl)-3H-quinazolin-4-one (CAS RN 1197495-53-6)

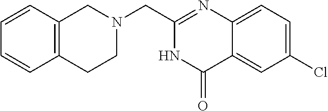 [SMILES: O=c1[nH]c(CN2CCc3ccccc3C2)nc2ccc(Cl)cc12]

### Example #59A. 6-chloro-2-(1,2,3,4-tetrahydroisoquinolin-2-ium-2-ylmethyl)-3H-quinazolin-4-one; chloride

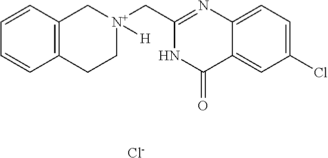 [SMILES: O=c1[nH]c(C[NH+]2CCc3ccccc3C2)nc2ccc(Cl)cc12 ; [Cl-]]

6-chloro-2-(3,4-dihydro-1H-isoquinolin-2-ylmethyl)-3H-quinazolin-4-one (152 mg; 0.46 mmol; 1.00 eq) was in suspension in 1,4-dioxane (130 mL). The reaction mixture was heated until complete solubilization. The reaction mixture was cooled at rt. HCl (4M in 1,4-dioxane) (116 μL; 0.46 mmol; 1 eq) was added. The reaction mixture was stirred at rt for 15 min (white precipitate was formed. The reaction mixture was concentrated under reduced pressure. The product was dried under vacuum at 50° C. for week-endDEJE1575-007 was dried under vacuum to provide 6-chloro-2-(1,2,3,4-tetrahydroisoquinolin-2-ium-2-ylmethyl)-3H-quinazolin-4-one (129.00 mg; 13%) as a white a morph powder. ¹H NMR (400 MHz, DMSO-d₆) δ=12.79 (br s, 1H), 11.20 (br s, 1H), 8.10 (d, J=2.5 Hz, 1H), 7.91 (dd, J=2.5, 8.7 Hz, 1H), 7.76 (d, J=8.7 Hz, 1H), 7.33-7.23 (m, 3H), 7.23-7.17 (m, 1H), 4.71-4.55 (m, 2H), 4.55-4.41 (m, 2H), 3.92-3.60 (m, 2H), 3.24-3.08 (m, 2H). LC/MS (Table 1, Method B) Rₜ=1.17 min; MS m/z: 326 [M+H]⁺.

### Example #60. 6-chloro-2-[(7-methyl-3,4-dihydro-1H-isoquinolin-2-yl)methyl]-3H-quinazolin-4-one

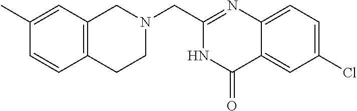 [SMILES: Cc1ccc2c(c1)CN(Cc1nc3ccc(Cl)cc3c(=O)[nH]1)CC2]

The Example #60 was synthesized using the same procedure as example #52. 1H NMR (DMSO-d6) δ: 12.18 (1H, s), 8.05 (1H, d), 7.84 (1H, dd), 7.70 (1H, d), 6.99 (1H, d), 6.93 (1H, dd), 6.84 (1H, d), 3.65 (2H, s), 3.62 (2H, s), 2.80 (4H, s), 2.22 (3H, s). MS (ES+): 340/342 (M+H)+.

### Example #61. 6-chloro-2-[(4-hydroxy-5,8-dimethoxy-3,4-dihydro-1H-isoquinolin-2-yl)methyl]-3H-quinazolin-4-one (CAS RN 1110903-31-5)

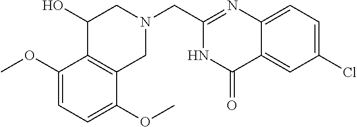 [SMILES: COc1ccc(OC)c2c1CN(Cc1nc3ccc(Cl)cc3c(=O)[nH]1)CC2O]

### Example #62. 6-chloro-2-(2,3,4,5-tetrahydro-1-benzazepin-1-ylmethyl)-3H-quinazolin-4-one

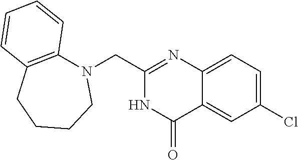 [SMILES: O=c1[nH]c(CN2CCCCc3ccccc32)nc2ccc(Cl)cc12]

2,3,4,5-tetrahydro-1H-benzo[b]azepine (0.072 g, 0.491 mmol) was added to a mixture of 6-chloro-2-(chloromethyl)quinazolin-4(3H)-one (0.075 g, 0.327 mmol) and potassium carbonate (0.136 g, 0.982 mmol) in EtOH (2 mL). The mixture was stirred at 75° C. overnight under nitrogen, allowed to cool to room temperature, then concentrated. The residue was diluted with sat. NaHCO₃ (10 mL) and DCM (15 mL) and filtered through a phase separator. The aqueous phase was extracted with DCM (10 mL) and the combined organics were washed with brine (15 mL), dried (MgSO₄) and absorbed onto celite. The crude product was purified by chromatography on silica gel (24 g cartridge, 0-50% EtOAc/isohexane+5% Et₃N) to afford 6-chloro-2-((2,3,4,5-tetrahydro-1H-benzo[b]azepin-1-yl)methyl)quinazolin-4(3H)-one (0.0312 g, 0.092 mmol, 28.0% yield) as a colourless solid. 1H NMR (DMSO, 500 MHz) δ 12.25 (1H, s), 8.04 (1H, d), 7.83 (1H, dd), 7.68 (1H, d), 7.16-7.03 (3H, m), 6.97 (1H, dd), 6.85 (1H, td), 4.27 (2H, s), 3.06-2.96 (2H, m), 2.91-2.83 (2H, m), 1.71-1.59 (2H, m), 1.59-1.47 (2H, m). MS (ES+): 340/342 (M+H)+

### Example #63. 6-chloro-2-[(1-methyl-3,4-dihydro-2H-1,5-benzodiazepin-5-yl)methyl]-3H-quinazolin-4-one

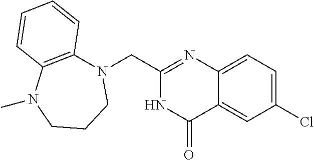 [SMILES: CN1CCCN(Cc2nc3ccc(Cl)cc3c(=O)[nH]2)c2ccccc21]

The Example #63 was synthesized using the same procedure as example #52. 1H NMR (DMSO-d6) δ: 12.17 (1H, s), 8.03 (1H, d), 7.81 (1H, dd), 7.64 (1H, d), 6.94 (1H, dd), 6.90-6.84 (2H, m), 6.80 (1H, ddd), 4.35 (2H, s), 3.25 (2H, t), 3.07 (2H, t), 2.84 (3H, s), 1.79-1.71 (2H, m). MS (ES+): 355/357 (M+H)+.

### Example #64. 6-chloro-2-(3,4-dihydro-2H-1,5-benzoxazepin-5-ylmethyl)-3H-quinazolin-4-one

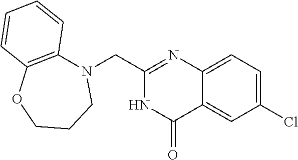 [SMILES: O=c1[nH]c(CN2CCCOc3ccccc32)nc2ccc(Cl)cc12]

The Example #64 was synthesized using the same procedure as example #52. 1H NMR (DMSO-d6) δ: 12.37 (1H, s), 8.05 (1H, d), 7.81 (1H, dd), 7.64 (1H, d), 6.88-6.81 (3H, m), 6.76-6.69 (1H, m), 4.35 (2H, s), 4.13 (2H, t), 3.40 (2H, dd), 2.01-1.92 (2H, m). MS (ES+): 342/344 (M+H)+.

### Example #73. 6-(dimethylamino)-2-(indolin-1-ylmethyl)-3H-quinazolin-4-one

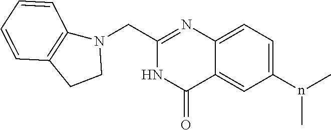

The compound was synthesized using the same procedure detailed in Example #2 starting from 2-(chloromethyl)-6-(dimethylamino)-3H-quinazolin-4-one (CAS RN 1690569-78-8, 205 mg; 0.86 mmol; 1.00 eq.) and indoline (CAS RN 496-15-1, 145 μL; 1.29 mmol; 1.50 eq.). The residue was purified by column chromatography (0 to 2% of MeOH in DCM) giving 6-(dimethylamino)-2-(indolin-1-ylmethyl)-3H-quinazolin-4-one (191 mg; 64%) as a beige amorphous powder. ¹H NMR (400 MHz, DMSO-d₆) δ=12.00 (br s, 1H), 7.51 (d, J=9.0 Hz, 1H), 7.31 (dd, J=3.0, 9.0 Hz, 1H), 7.18 (d, J=3.0 Hz, 1H), 7.05 (dd, J=1.2, 7.6 Hz, 1H), 6.97 (dt, J=1.2, 7.6 Hz, 1H), 6.64-6.57 (m, 2H), 4.13 (s, 2H), 3.47 (t, J=8.3 Hz, 2H), 3.00 (s, 6H), 2.92 (t, J=8.3 Hz, 2H). LC/MS (Table 1, Method B) Rₜ=1.46 min; MS m/z: 321 [M+H]⁺.

### Example #74. 6-(dimethylamino)-2-[(3,3-dimethylindolin-1-yl)methyl]-3H-quinazolin-4-one

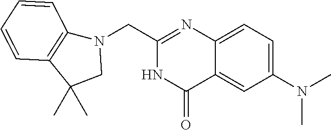 [SMILES: CN(C)c1ccc2nc(CN3CC(C)(C)c4ccccc43)[nH]c(=O)c2c1]

The compound was synthesized using the same procedure detailed in Example #2 starting from 2-(chloromethyl)-6-(dimethylamino)-3H-quinazolin-4-one (CAS RN 1690569-78-8, 150 mg; 0.63 mmol; 1.00 eq.) and 3,3-dimethylindoline (CAS RN 1914-02-9, 139 mg; 0.95 mmol; 1.50 eq.). The residue was purified by column chromatography (5 to 40% of EtOAc in DCM) giving 6-(dimethylamino)-2-[(3,3-dimethylindolin-1-yl)methyl]-3H-quinazolin-4-one (50 mg; 23%) as a yellow Amorphous powder. ¹H NMR (400 MHz, DMSO-d₆) δ=12.01 (br s, 1H), 7.50 (d, J=9.0 Hz, 1H), 7.30 (dd, J=3.0, 9.0 Hz, 1H), 7.18 (d, J=3.0 Hz, 1H), 7.01 (dd, J=0.8, 7.3 Hz), 6.98 (td, J=1.2, 7.7 Hz, 1H), 6.63 (td, J=0.8, 7.3 Hz, 1H) 6.62 (d, J=7.7 Hz, 1H), 4.13 (s, 2H), 3.25 (s, 2H), 3.00 (s, 6H), 1.25 (s, 6H). LC/MS (Table 1, Method B) Rₜ=1.68 min; MS m/z: 349 [M+H]⁺.

### Example #76. 7-fluoro-2-(indolin-1-ylmethyl)-6-methoxy-3H-quinazolin-4-one

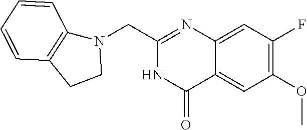 [SMILES: COc1cc2c(=O)[nH]c(CN3CCc4ccccc43)nc2cc1F]

The compound was synthesized using the same procedure detailed in Example #2 starting from 2-(chloromethyl)-7-fluoro-6-methoxy-3H-quinazolin-4-one (CAS RN 1700039-84-4, 150 mg; 0.61 mmol; 1.00 eq.) and indoline (CAS RN 496-15-1, 102 μL; 0.91 mmol; 1.50 eq.) giving 7-fluoro-2-(indolin-1-ylmethyl)-6-methoxy-3H-quinazolin-4-one (158 mg; 77%) as a white amorphous powder. ¹H NMR (400 MHz, DMSO-d₆) δ=12.34 (br s, 1H), 7.66 (d, J=9.4 Hz, 1H), 7.51 (d, J=12.1 Hz, 1H), 7.06 (dd, J=0.8, 7.3 Hz, 1H), 6.98 (t, J=7.3 Hz, 1H), 6.61 (td, J=0.8, 7.3 Hz, 1H), 6.58 (d, J=8.5 Hz, 1H), 4.17 (s, 2H), 3.95 (s, 3H), 3.49 (t, J=8.3 Hz, 2H), 2.93 (t, J=8.3 Hz, 2H). LC/MS (Table 1, Method B) Rₜ=1.51 min; MS m/z: 326 [M+H]⁺.

### Example #77. 2-[(3,3-dimethylindolin-1-yl)methyl]-7-fluoro-6-methoxy-3H-quinazolin-4-one

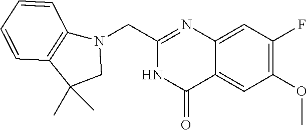 [SMILES: COc1cc2c(=O)[nH]c(CN3CC(C)(C)c4ccccc43)nc2cc1F]

The compound was synthesized using the same procedure detailed in example #2 starting from 2-(chloromethyl)-7-fluoro-6-methoxy-3H-quinazolin-4-one (CAS RN 1700039-84-4, 150 mg; 0.61 mmol; 1.00 eq.) and 3,3-dimethylindoline (CAS RN 1914-02-9, 137 mg; 0.93 mmol; 1.50 eq.). The residue was purified by column chromatography (0 to 20% of EtOAc in DCM) then triturated in ACN to give 2-[(3,3-dimethylindolin-1-yl)methyl]-7-fluoro-6-methoxy-3H-quinazolin-4-one (71 mg; 32.5%) as a white amorphous powder. mp=194-209° C. ¹H NMR (400 MHz, DMSO-d₆) δ=12.37 (br s, 1H), 7.67 (d, J=9.4 Hz, 1H), 7.50 (d, J=12.1 Hz, 1H), 7.02 (d, J=7.2 Hz, 1H), 6.98 (td, J=0.9, 7.5 Hz, 1H), 6.64 (t, J=7.4 Hz 1H), 6.60 (d, J=7.8 Hz, 1H), 4.17 (s, 2H), 3.96 (s, 3H), 3.26 (s, 2H), 1.25 (s, 6H). LC/MS (Table 1, Method B) R₁=1.72 min; MS m/z: 354 [M+H]⁺.

### Example #78. 2-(indolin-1-ylmethyl)-6-methoxy-3H-pyrido[3,4-d]pyrimidin-4-one

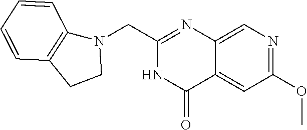 [SMILES: COc1cc2c(=O)[nH]c(CN3CCc4ccccc43)nc2cn1]

The compound was synthesized using the same procedure detailed in Example #2 starting from 2-(chloromethyl)-6-methoxy-3H-pyrido[3,4-d]pyrimidin-4-one (preparation #5, 212 mg, 0.94 mmol, 1.00 eq.) and indoline (CAS RN 496-15-1, 210 μL; 1.87 mmol; 1.99 eq.) to give 2-(indolin-1-ylmethyl)-6-methoxy-3H-pyrido[3,4-d]pyrimidin-4-one (206 mg; 71%) as a yellow amorphous powder. mp=215-230° C. ¹H NMR (400 MHz, DMSO-d₆) δ=12.44 (s, 1H), 8.73 (d, J=0.8 Hz, 1H), 7.28 (d, J=0.8 Hz, 1H), 7.06 (dd, J=0.7, 7.2 Hz, 1H), 6.98 (dt, J=1.2, 7.7 Hz, 1H), 6.62 (td, J=0.9, 7.2 HZ, 1H) 6.60 (d, J=7.7 HZ, 1H), 4.18 (s, 2H), 3.94 (s, 3H), 3.49 (t, J=8.3 Hz, 2H), 2.93 (t, J=8.3 Hz, 2H). LC/MS (Table 1, Method B) Rₜ=1.39 min; MS m/z: 309 [M+H]⁺.

### Example #79. 2-[(3,3-dimethylindolin-1-yl)methyl]-6-methoxy-3H-pyrido[3,4-d]pyrimidin-4-one

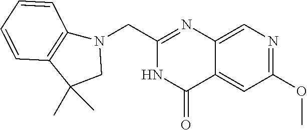 [SMILES: COc1cc2c(=O)[nH]c(CN3CC(C)(C)c4ccccc43)nc2cn1]

The compound was synthesized using the same procedure detailed in Example #2 starting from 2-(chloromethyl)-6-methoxy-3H-pyrido[3,4-d]pyrimidin-4-one (preparation #5, 185 mg, 0.82 mmol, 1.00 eq.) and 3,3-dimethylindoline (CAS RN 1914-02-9, 181 mg; 1.23 mmol; 1.50 eq.). The residue was purified by column chromatography (0 to 15% of EtOAc in DCM) giving 2-[(3,3-dimethylindolin-1-yl)methyl]-6-methoxy-3H-pyrido[3,4-d]pyrimidin-4-one (125 mg; 45%) as a white amorphous powder. mp=145-167° C. ¹H NMR (400 MHz, DMSO-d₆) δ=12.46 (s, 1H), 8.72 (d, J=0.8 Hz, 1H), 7.28 (d, J=0.8 Hz, 1H), 7.03 (dd, J=0.9, 7.3 Hz, 1H), 6.99 (dt, J=1.3, 7.6 Hz, 1H), 6.65 (dt, J=0.9, 7.3 Hz, 1H), 6.62 (d, J=7.9 Hz, 1H), 4.18 (s, 2H), 3.94 (s, 3H), 3.26 (s, 2H), 1.25 (s, 6H). LC/MS (Table 1, Method B) Rₜ=1.63 min; MS m/z: 337 [M+H]⁺.

### Example #80. 6-(dimethylamino)-7-fluoro-2-(indolin-1-ylmethyl)-3H-quinazolin-4-one

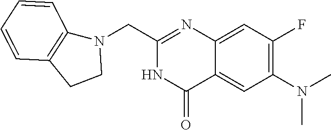 [SMILES: CN(C)c1cc2c(=O)[nH]c(CN3CCc4ccccc43)nc2cc1F]

### Step A: 2-(chloromethyl)-6-(dimethylamino)-7-fluoro-3H-quinazolin-4-one

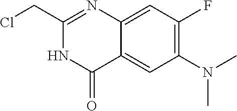 [SMILES: CN(C)c1cc2c(=O)[nH]c(CCl)nc2cc1F]

Chloroacetonitrile (CAS RN 107-14-2, 141 μL; 2.23 mmol; 3.00 eq.) was dissolved in MeOH (1.50 mL). At 10° C., sodium methoxide (8 mg; 0.15 mmol; 0.20 eq.) was added. The reaction mixture was stirred Under N2 atm, at 10° C. for 1 h10. A solution of 2-amino-5-(dimethylamino)-4-fluoro-benzoic acid (CAS RN 1247552-18-6, 147 mg; 0.74 mmol; 1.00 eq.) in MeOH (3.00 mL) was added slowly. The reaction mixture was stirred at rt for 1 h30. The reaction mixture was filtered through sintered filter funnel, washed with water three times, then with cold MeOH. The solid was dried under vacuo at 50° C. overnight to give 2-(chloromethyl)-6-(dimethylamino)-7-fluoro-3H-quinazolin-4-one (110 mg; 58%). ¹H NMR (300 MHz, DMSO-ds) δ=12.54 (br s, 1H), 7.47 (d, J=9.7 Hz, 1H), 7.44 (d, J=14.2 Hz, 1H), 4.52 (s, 2H), 2.88 (d, J=1.2 Hz, 6H). LC/MS (Table 1, Method A) Rₜ=2.04 min; MS m/z: 256 [M+H]⁺.

### Step B: 6-(dimethylamino)-7-fluoro-2-(indolin-1-ylmethyl)-3H-quinazolin-4-one

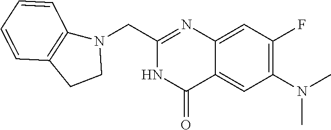 [SMILES: CN(C)c1cc2c(=O)[nH]c(CN3CCc4ccccc43)nc2cc1F]

The compound was synthesized using the same procedure detailed in Example #2 starting from 2-(chloromethyl)-6-(dimethylamino)-7-fluoro-3H-quinazolin-4-one (Example #80, Step A) (100 mg, 0.39 mmol, 1.00 eq.) and indoline (CAS RN 496-15-1, 66 μL, 0.59 mmol, 1.50 eq.). The residue was purified by column chromatography (5 to 50% of EtOAc in cyclohexane) giving 6-(dimethylamino)-7-fluoro-2-(indolin-1-ylmethyl)-3H-quinazolin-4-one (86 mg; 65%) as a white amorphous powder. mp=200-223° C. ¹H NMR (400 MHz, DMSO-d₆) δ=12.22 (br s, 1H), 7.48 (d, J=9.8 Hz, 1H), 7.39 (d, J=14.1 Hz, 1H), 7.05 (dd, J=0.8, 7.2 Hz, 1H), 6.97 (dt, J=1.2, 7.7 Hz, 1H), 6.61 (dt, J=0.8, 7.4 Hz, 1H), 6.57 (d, J=7.8 Hz, 1H), 4.16 (s, 2H), 3.48 (t, J=8.3 Hz, 2H), 2.92 (t, J=8.3 Hz, 2H), 2.86 (d, J=0.7 Hz, 6H). LC/MS (Table 1, Method B) Rₜ=1.58 min; MS m/z: 339 [M+H]⁺.

### Example #81. 2-(indolin-1-ylmethyl)-6-isopropyl-3H-quinazolin-4-one

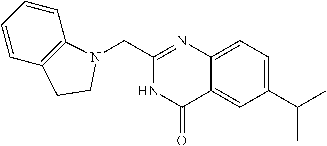 [SMILES: CC(C)c1ccc2nc(CN3CCc4ccccc43)[nH]c(=O)c2c1]

The compound was synthesized using the same procedure detailed in Example #1 starting from 2-(chloromethyl)-6-isopropyl-3H-quinazolin-4-one (CAS RN 1691892-87-1, 202 mg, 0.85 mmol, 1.00 eq.) and indoline (CAS RN 496-15-1, 110 μL; 1.02 mmol; 1.20 eq.) to give 2-(indolin-1-ylmethyl)-6-isopropyl-3H-quinazolin-4-one (132 mg; 48%) as a white amorphous powder. mp=168-185° C. ¹H NMR (400 MHz, DMSO-d₆) δ=12.19 (br s, 1H), 7.94 (d, J=2.2 Hz, 1H), 7.71 (dd, J=2.2, 8.5 Hz, 1H), 7.58 (d, J=8.5 Hz, 1H), 7.06 (dd, J=0.7, 7.2 Hz, 1H), 6.97 (dt, J=1.2, 7.7 Hz, 1H), 6.61 (dt, J=0.8, 7.3 Hz, 1H) 6.58 (d, J=7.7 Hz, 1H), 4.18 (s, 2H), 3.49 (t, J=8.3 Hz, 2H), 3.05 (quin, J=6.8 Hz, 1H), 2.93 (t, J=8.3 Hz, 2H), 1.25 (d, J=6.8 Hz, 6H). LC/MS (Table 1, Method B) Rₜ=1.72 min; MS m/z: 320 [M+H]⁺.

### Example #82. 2-(indolin-1-ylmethyl)-6-(trifluoromethoxy)-3H-quinazolin-4-one

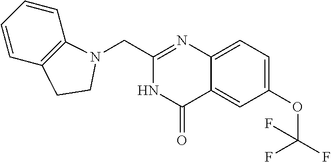 [SMILES: O=c1[nH]c(CN2CCc3ccccc32)nc2ccc(OC(F)(F)F)cc12]

The compound was synthesized using the same procedure detailed in Example #2 starting from 2-(chloromethyl)-6-(trifluoromethoxy)-3H-quinazolin-4-one (CAS RN 1831080-35-3, 160 mg; 0.57 mmol; 1.00 eq.) and indoline (CAS RN 496-15-1, 160 mg; 0.57 mmol; 1.00 eq.). The residue was purified by column chromatography (0 to 20% of EtOAc in DCM) giving 2-(indolin-1-ylmethyl)-6-(trifluoromethoxy)-3H-quinazolin-4-one (99 mg; 47.5%) as a pink amorphous powder. ¹H NMR (400 MHz, DMSO-d₆) δ=12.55 (br s, 1H), 7.95 (td, J=1.1, 2.2 Hz, 1H), 7.84-7.76 (m, 2H), 7.06 (dd, J=0.6, 7.2 Hz, 1H), 6.98 (dt, J=1.2, 7.7 Hz, 1H), 6.65-6.57 (m, 2H), 4.22 (s, 2H), 3.50 (t, J=8.3 Hz, 2H), 2.94 (t, J=8.3 Hz, 2H). LC/MS (Table 1, Method B) Rₜ=1.72 min; MS m/z: 362 [M+H]⁺.

### Example #83. 2-(indolin-1-ylmethyl)-6,7-dimethoxy-3H-quinazolin-4-one (CAS RN 784172-90-3)

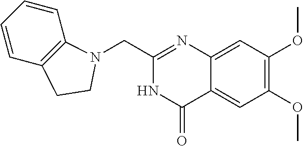 [SMILES: COc1cc2nc(CN3CCc4ccccc43)[nH]c(=O)c2cc1OC]

### Example #84. 2-(indolin-1-ylmethyl)-6-methyl-3H-quinazolin-4-one

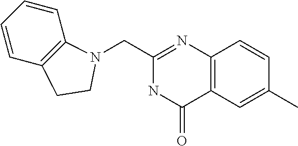 [SMILES: Cc1ccc2nc(CN3CCc4ccccc43)[nH]c(=O)c2c1]

The compound was synthesized using the same procedure detailed in Example #1 starting from 2-(chloromethyl)-6-methyl-3H-quinazolin-4-one (CAS RN 18731-18-5, 119 mg, 0.57 mmol, 1.00 eq.) and indoline (CAS RN 496-15-1, 70 μL; 0.63 mmol; 1.10 eq.). The residue was purified by column chromatography (0 to 30% of EtOAc in cyclohexane) giving 2-(indolin-1-ylmethyl)-6-methyl-3H-quinazolin-4-one (110 mg; 66%) as a white amorphous powder. ¹H NMR (400 MHz, DMSO-d₆) δ=12.16 (br s, 1H), 7.90 (br s, 1H), 7.62 (dd, J=2.0, 8.5 Hz, 1H), 7.54 (d, J=8.3 Hz, 1H), 7.06 (d, J=8.2 Hz, 1H), 6.97 (dt, J=1.2, 7.7 Hz, 1H), 6.61 (t, J=7.3 Hz, 1H) 6.59 (d, J=7.8 Hz, 1H), 4.18 (s, 2H), 3.49 (t, J=8.3 Hz, 2H), 2.93 (t, J=8.3 Hz, 2H), 2.44 (s, 3H). LC/MS (Table 1, Method B) Rₜ=1.54 min; MS m/z: 292 [M+H]⁺.

### Example #85. 2-(indolin-1-ylmethyl)-6-(trifluoromethyl)-3H-quinazolin-4-one

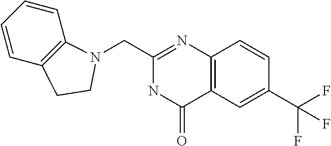 [SMILES: O=c1[nH]c(CN2CCc3ccccc32)nc2ccc(C(F)(F)F)cc12]

A suspension of 2-(chloromethyl)-6-(trifluoromethyl)quinazolin-4(3H)-one (0.060 g, 0.227 mmol) in MeCN (2.5 mL) was treated with indoline (0.077 ml, 0.681 mmol) and the resulting suspension was stirred at 50° C. for 18 h. The reaction mixture was concentrated in vacuo and the crude product was purified by preparative HPLC (Waters, Basic (0.1% Ammonium Bicarbonate), Basic, Waters X-Bridge Prep-C18, 5 μm, 19×50 mm column, 35-65% MeCN in Water) to afford 2-(indolin-1-ylmethyl)-6-(trifluoromethyl)quinazolin-4(3H)-one (22.2 mg, 27.8%) as a pale brown solid. 1H NMR (500 MHz, DMSO-d6) δ 12.63 (br s, 1H), 8.35 (d, 1H), 8.10 (dd, 1H), 7.83 (d, 1H), 7.09-7.05 (m, 1H), 7.00-6.96 (m, 1H), 6.64-6.60 (m, 1H), 6.59 (d, 1H), 4.25 (s, 2H), 3.52 (t, 2H), 2.95 (t, 2H). MS (ES+): 346/348 (M+H)+.

### Example #86. 2-(indolin-1-ylmethyl)-7-methoxy-3H-quinazolin-4-one

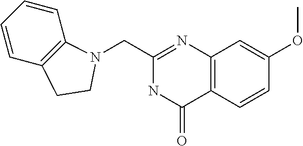 [SMILES: COc1ccc2c(=O)[nH]c(CN3CCc4ccccc43)nc2c1]

The compound was synthesized using the same procedure detailed in Example #1 starting from 2-(chloromethyl)-7-methoxy-3H-quinazolin-4-one (CAS RN 1257043-54-1, 136 mg, 0.61 mmol, 1.00 eq.) and indoline (CAS RN 496-15-1, 70 μL, 0.67 mmol, 1.10 eq.). The residue was purified by column chromatography (0 to 30% of EtOAc in cyclohexane) giving 2-(indolin-1-ylmethyl)-7-methoxy-3H-quinazolin-4-one (83 mg; 45%) as a grey amorphous powder. 11H NMR (400 MHz, DMSO-d₆) δ=12.10 (s, 1H), 8.00 (dd, J=0.5, 8.5 Hz, 1H), 7.10-7.04 (m, 3H), 6.98 (dt, J=1.2, 7.6 Hz, 1H), 6.64-6.56 (m, 2H), 4.18 (s, 2H), 3.88 (s, 3H), 3.51 (t, J=8.3 Hz, 2H), 2.94 (t, J=8.3 Hz, 2H). LC/MS (Table 1, Method B) Rₜ=1.42 min; MS m/z: 308 [M+H]⁺.

### Example #87. 2-(indolin-1-ylmethyl)-6-isopropoxy-3H-quinazolin-4-one

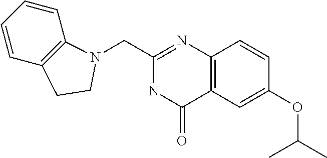 [SMILES: CC(C)Oc1ccc2nc(CN3CCc4ccccc43)[nH]c(=O)c2c1]

### Step A: 2-(chloromethyl)-6-isopropoxy-3H-quinazolin-4-one

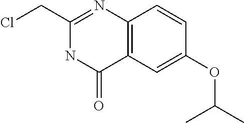 [SMILES: CC(C)Oc1ccc2nc(CCl)[nH]c(=O)c2c1]

The compound was synthesized using the same procedure detailed in preparation #5 starting from 2-amino-5-isopropoxy-benzoic acid (CAS RN 68701-42-8, 200 mg; 0.86 mmol; 1.00 eq.) and chloroacetonitrile (CAS RN 107-14-2, 164 μL; 2.59 mmol; 3.00 eq.) to give 2-(chloromethyl)-6-isopropoxy-3H-quinazolin-4-one (130 mg; 57%) as a beige amorphous powder. 11H NMR (300 MHz, DMSO-d₆) δ=12.51 (br s, 1H), 7.62 (d, J=8.9 Hz, 1H), 7.49 (d, J=3.0 Hz, 1H), 7.40 (dd, J=3.0, 8.9 Hz, 1H), 4.75 (quin, J=6.0 Hz, 1H), 4.53 (s, 2H), 1.32 (d, J=6.0 Hz, 6H). LC/MS (Table 1, Method A) Rₜ=2.17 min; MS m/z: 253 [M+H]⁺.

### Step B: 2-(indolin-1-ylmethyl)-6-isopropoxy-3H-quinazolin-4-one

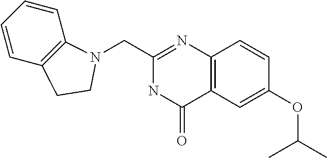 [SMILES: CC(C)Oc1ccc2nc(CN3CCc4ccccc43)[nH]c(=O)c2c1]

The compound was synthesized using the same procedure detailed in Example #2 starting from 2-(chloromethyl)-6-isopropoxy-3H-quinazolin-4-one (Example #87, Step A) (123 mg; 0.47 mmol; 1.00 eq.) and indoline (CAS RN 496-15-1, 79 μL; 0.70 mmol; 1.50 eq.). The residue was purified by column chromatography (5 to 40% of EtOAc in cyclohexane) giving 2-(indolin-1-ylmethyl)-6-isopropoxy-3H-quinazolin-4-one (65 mg; 41%) as a beige amorphous powder. 1H NMR (400 MHz, DMSO-d₆) δ=12.21 (s, 1H), 7.58 (d, J=8.9 Hz, 1H), 7.47 (d, J=3.0 Hz, 1H), 7.36 (dd, J=3.0, 8.9 Hz, 1H), 7.05 (d, J=7.3 Hz, 1H), 6.98 (dt, J=1.2, 7.6 Hz, 1H), 6.64-6.57 (m, 2H), 4.73 (quin, J=6.0 Hz, 1H), 4.16 (s, 2H), 3.48 (t, J=8.3 Hz, 2H), 2.92 (t, J=8.3 Hz, 2H), 1.31 (d, J=6.0 Hz, 6H). LC/MS (Table 1, Method B) Rₜ=1.64 min; MS m/z: 336 [M+H]⁺.

### Example #88. 6-fluoro-2-(indolin-1-ylmethyl)-3H-quinazolin-4-one

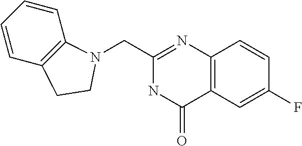 [SMILES: O=c1[nH]c(CN2CCc3ccccc32)nc2ccc(F)cc12]

The compound was synthesized using the same procedure detailed in Example #2 starting from 2-(chloromethyl)-6-fluoro-3H-quinazolin-4-one (CAS RN 163311-08-8, 150 mg; 0.71 mmol; 1.00 eq.) and indoline (CAS RN 496-15-1, 119 μL; 1.06 mmol; 1.50 eq.). The residue was purified by column chromatography (5 to 40% of EtOAc in cyclohexane) giving 6-fluoro-2-(indolin-1-ylmethyl)-3H-quinazolin-4-one (104 mg; 50%) as a beige amorphous powder. mp=184-196° C. ¹H NMR (400 MHz, DMSO-d₆) δ=12.41 (br s, 1H), 7.78 (ddd, J=0.7, 2.9, 8.7 Hz, 1H), 7.75-7.66 (m, 2H), 7.08-7.04 (m, 1H), 6.98 (dt, J=1.2, 7.6 Hz, 1H), 6.61 (td, J=1.2, 7.6 Hz, 1H) 6.59 (d, J=7.6 Hz, 1H), 4.20 (s, 2H). LC/MS (Table 1, Method B) Rₜ=1.42 min; MS m/z: 296 [M+H]⁺.

### Example #89. 7-chloro-2-(indolin-1-ylmethyl)-3H-quinazolin-4-one (CAS RN 849054-31-5)

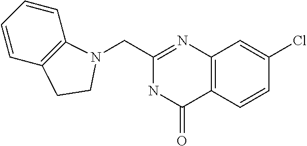 [SMILES: O=c1[nH]c(CN2CCc3ccccc32)nc2cc(Cl)ccc12]

### Example #90. tert-butyl N-[4-[[2-(indolin-1-ylmethyl)-4-oxo-3H-quinazolin-6-yl]oxy]butyl]carbamate

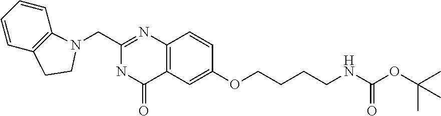 [SMILES: CC(C)(C)OC(=O)NCCCCOc1ccc2nc(CN3CCc4ccccc43)[nH]c(=O)c2c1]

### Step A: 6-hydroxy-2-(indolin-1-ylmethyl)-3H-quinazolin-4-one

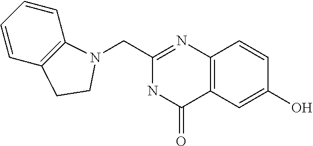 [SMILES: O=c1[nH]c(CN2CCc3ccccc32)nc2ccc(O)cc12]

2-(chloromethyl)-6-hydroxy-3H-quinazolin-4-one (CAS RN 1258977-05-7, 557 mg; 2.64 mmol; 1.00 eq.) and indoline (CAS RN 496-15-1, 445 μL; 3.97 mmol; 1.50 eq.) were dissolved in THF (11 mL). DIPEA (655 μL; 3.96 mmol; 1.50 eq.) was added and mixture was stirred at 70° C. overnight. 30 ml of water were added and the mixture was acidified until pH 6 with aq. HCl (1N). It was filtered, washed three times with water, three times with Et₂O, and dried under vacuum at rt. The residue was purified by column chromatography (20 to 70% of EtOAc in cyclohexane) giving 6-hydroxy-2-(indolin-1-ylmethyl)-3H-quinazolin-4-one (180 mg; 22%) as a beige amorphous powder. ¹H NMR (DMSO-d₆) δ: 12.05 (br s, 1H), 10.03 (s, 1H), 7.51 (d, J=8.8 Hz, 1H), 7.39 (d, J=2.8 Hz, 1H), 7.24 (dd, J=2.8, 8.8 Hz, 1H), 7.05 (dd, J=1.1, 7.7 Hz, 1H), 6.97 (td, J=1.2, 7.6 Hz, 1H), 6.57-6.64 (m, 2H), 4.14 (s, 2H), 3.47 (t, J=8.3 Hz, 2H), 2.92 (t, J=8.3 Hz, 2H). LC/MS (Table 1, Method B) Rₜ=1.22 min; MS m/z: 294 [M+H]⁺.

### Step B: tert-butyl N-[4-[[2-(indolin-1-ylmethyl)-4-oxo-3H-quinazolin-6-yl]oxy]butyl]carbamate

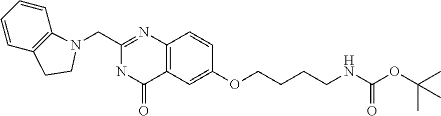 [SMILES: CC(C)(C)OC(=O)NCCCCOc1ccc2nc(CN3CCc4ccccc43)[nH]c(=O)c2c1]

PPh₃ (349 mg; 1.33 mmol; 1.30 eq.) was dissolved in THF (1.80 mL). DIAD (265 μL; 1.34 mmol; 1.31 eq.) was added dropwise at rt and the mixture was stirred under N₂ atm, at rt for 5 min providing a yellow solid. The heterogeneous mixture was dissolved by adding THF (1.00 mL) and this solution was added onto a solution of 6-hydroxy-2-(indolin-1-ylmethyl)-3H-quinazolin-4-one (Example #90, Step A, 300 mg; 1.02 mmol; 1.00 eq.) and 4-(boc-amino)-1-butanol (CAS RN 75178-87-9, 252 mg; 1.33 mmol; 1.30 eq.) in THF (3 mL) at rt. The resulting brown cloudy mixture was stirred at rt for 3 days. PPh₃ (349 mg; 1.33 mmol; 1.30 eq.) was dissolved in THF (1.80 mL). DIAD (265 μL; 1.34 mmol; 1.31 eq.) was added dropwise and mixture was stirred at rt for 15 min to give a yellow solid. The heterogeneous mixture was dissolved by adding THF (1.00 mL) and this solution was added onto the previous reaction mixture and stirred at rt for 24 h more. THF was evaporated and residue was purified by column chromatography (20 to 50% of EtOAc in cyclohexane) then recrystallized with DMSO and ACN to give tert-butyl N-[4-[[2-(indolin-1-ylmethyl)-4-oxo-3H-quinazolin-6-yl]oxy]butyl]carbamate (38 mg; 8%) as a white amorphous powder mp=167-181° C. ¹H NMR (DMSO-d₆) δ: 12.20 (s, 1H), 7.58 (d, J=8.9 Hz, 1H), 7.48 (d, J=3.0 Hz, 1H), 7.38 (dd, J=3.0, 8.9 Hz, 1H), 7.06 (dd, J=0.6, 7.2 Hz, 1H), 6.97 (td, J=1.0, 7.7 Hz, 1H), 6.85 (br t, J=5.6 Hz, 1H), 6.57-6.65 (m, 2H), 4.17 (s, 2H), 4.07 (t, J=6.6 Hz, 2H), 3.48 (t, J=8.3 Hz, 2H), 2.98 (q, J=6.6 Hz, 2H), 2.93 (t, J=8.3 Hz, 2H), 1.69-1.78 (m, 2H), 1.54 (quin, J=7.2 Hz, 2H), 1.37 (s, 9H). LC/MS (Table 1, Method B) Rₜ=1.96 min; MS m/z: 465 [M+H]⁺.

### Example #91. 2-(indolin-1-ylmethyl)-7-(trifluoromethyl)-3H-quinazolin-4-one

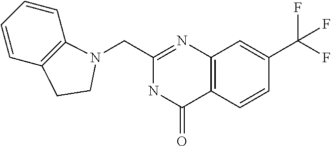 [SMILES: O=c1[nH]c(CN2CCc3ccccc32)nc2cc(C(F)(F)F)ccc12]

The compound was synthesized using the same procedure detailed in Example #1 starting from 2-(chloromethyl)-7-(trifluoromethyl)-3H-quinazolin-4-one (CAS RN 730976-60-0, 184 mg; 0.70 mmol; 1.00 eq.) and indoline (CAS RN 496-15-1, 90 μL; 0.77 mmol; 1.10 eq.). The residue was purified by column chromatography (0 to 30% of EtOAc in cyclohexane) giving 2-(indolin-1-ylmethyl)-7-(trifluoromethyl)-3H-quinazolin-4-one (80 mg; 32%) as a beige amorphous powder. ¹H NMR (400 MHz, DMSO-d₆) δ=12.59 (br s, 1H), 8.31 (d, J=8.3 Hz, 1H), 7.96 (s, 1H), 7.80 (dd, J=1.7, 8.4 Hz, 1H), 7.07 (dd, J=0.7, 7.2 Hz, 1H), 6.98 (dt, J=1.1, 7.6 Hz, 1H), 6.62 (td, J=0.9, 7.3 Hz, 1H) 6.60 (d, J=7.9 Hz, 1H), 4.24 (s, 2H), 3.52 (t, J=8.3 Hz, 2H), 2.95 (t, J=8.3 Hz, 2H). LC/MS (Table 1, Method B) Rₜ=1.79 min; MS m/z: 346 [M+H]⁺.

### Example #92. 6-(dimethylamino)-2-[(3,3-dimethylindolin-1-yl)methyl]-3H-pyrido[3,4-d]pyrimidin-4-one

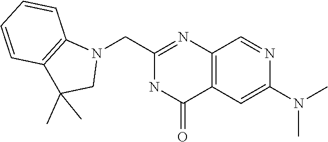

### Step A: 5-(tert-butoxycarbonylamino)-2-(dimethylamino)pyridine-4-carboxylic acid

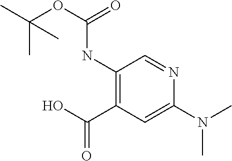

tert-Butyl N-[6-(dimethylamino)-3-pyridyl]carbamate (CAS RN 1242333-49-8, 320 mg; 1.35 mmol; 1.00 eq.) was dissolved in Et₂O (12 mL). TMEDA (650 μL; 4.31 mmol; 3.20 eq.) was added. The reaction mixture was cold at −78° C. n-BuLi (1.6M in hexane) (3.60 mL; 1.60 mol/L; 5.76 mmol; 4.30 eq.) was added drop wise. The reaction mixture was allowed to warm at −10° C. and the reaction mixture was stirred at −10° C. for 2h. CO₂ (s) (dry ice) was dried with MgSO₄. The released CO₂ gas was bubbled into the reaction mixture using a cannula for 2h. The reaction mixture was allowed to warm at rt, washed with NH₄Cl and extracted with EtOAc twice. The aqueous layer was acidified with HCl 1N and extracted with EtOAc. The organic layer was dried over MgSO₄, filtered and concentrated. The residue was purified by column chromatography (0 to 10% of MeOH in DCM) giving 5-(tert-butoxycarbonylamino)-2-(dimethylamino)pyridine-4-carboxylic acid (93 mg; 24.5%) as a yellow amorphous powder. ¹H NMR (300 MHz, DMSO-d₆) δ=11.30 (br s, 1H), 8.78 (br s, 1H), 7.09 (s, 1H), 2.96 (s, 6H), 1.45 (s, 9H). LC/MS (Table 1, Method A) Rₜ=1.70 min; MS m/z: 282 [M+H]⁺.

### Step B: 5-amino-2-(dimethylamino)pyridin-1-ium-4-carboxylic acid; 2,2,2-trifluoroacetate

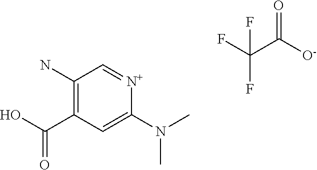

5-(tert-butoxycarbonylamino)-2-(dimethylamino)pyridine-4-carboxylic acid (Example #92, Step A, 90 mg; 0.32 mmol; 1.00 eq.) was suspended in DCM (2 mL). TFA (2 mL) was added (yellow solution) at rt. The reaction mixture was stirred at rt for 50 min. The reaction mixture was concentrated under reduced pressure to give 5-amino-2-(dimethylamino)pyridin-1-ium-4-carboxylic acid 2,2,2-trifluoroacetate (108 mg; quantitative) as a yellow powder. ¹H NMR (300 MHz, DMSO-d₆) δ=7.97 (s, 1H), 7.13 (s, 1H), 3.02 (s, 6H).

### Step C: 2-(chloromethyl)-6-(dimethylamino)-3H-pyrido[3,4-d]pyrimidin-4-one

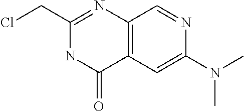

The compound was prepared using the same procedure detailed in Preparation #5 starting from 5-amino-2-(dimethylamino)pyridin-1-ium-4-carboxylic acid 2,2,2-trifluoroacetate (Example #92, Step B, 108 mg; 0.60 mmol; 1.00 eq.) and chloroacetonitrile (CAS RN 107-14-2, 114 μL; 1.79 mmol; 3.00 eq.) to give 2-(chloromethyl)-6-(dimethylamino)-3H-pyrido[3,4-d]pyrimidin-4-one (25 mg; 18%) as a beige amorphous powder. ¹H NMR (300 MHz, DMSO-d₆) δ=12.44 (br s, 1H), 8.66 (d, J=0.8 Hz, 1H), 6.95 (d, J=0.8 Hz, 1H), 4.51 (s, 3H), 3.12 (s, 6H). LC/MS (Table 1, Method A) Rₜ=1.84 min; MS m/z: 239 [M+H]⁺.

### Step D: 6-(dimethylamino)-2-[(3,3-dimethylindolin-1-yl)methyl]-3H-pyrido[3,4-d]pyrimidin-4-one

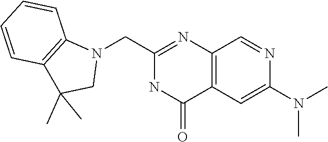

A mixture of 2-(chloromethyl)-6-(dimethylamino)-3H-pyrido[3,4-d]pyrimidin-4-one Example #92, Step C (30 mg, 0.13 mmol, 1.00 eq.), 3,3-dimethylindoline (CAS RN 1914-02-9, 56 mg; 0.38 mmol; 3.0 eq.) and K₂CO₃ (52 mg; 0.38 mmol; 3.00 eq.) in EtOH (0.9 mL) was stirred at 80° C. overnight. After cooling down to rt, the suspension was filtered and washed with MeOH. The filtrate was concentrated to dryness. The crude was purified by column chromatography (5 to 40% of EtOAc in cyclohexane) giving 6-(dimethylamino)-2-[(3,3-dimethylindolin-1-yl)methyl]-3H-pyrido[3,4-d]pyrimidin-4-one (31 mg; 71%) as a white amorphous powder. mp=194-211° C. ¹H NMR (400 MHz, DMSO-d₆) δ=12.19 (br s, 1H), 8.63 (d, J=0.9 Hz, 1H), 7.02 (dd, J=0.8, 7.2 Hz, 1H) 6.99 (td, J=1.3, 7.6 Hz, 1H), 6.95 (d, J=0.8 Hz, 1H), 6.65 (td, J=0.8, 7.4 Hz, 1H) 6.62 (d, J=7.9 Hz, 1H), 4.12 (s, 2H), 3.24 (s, 2H), 3.11 (s, 6H), 1.25 (s, 6H). LC/MS (Table 1, Method B) Rₜ=1.66 min; MS m/z: 350 [M+H]⁺.

### Example #93. 7-chloro-2-[(3,3-dimethylindolin-1-yl)methyl]-6-methoxy-3H-quinazolin-4-one

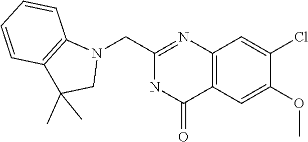

### Step A: 7-chloro-2-(chloromethyl)-6-methoxy-3H-quinazolin-4-one

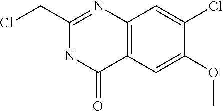

The compound was prepared using the same procedure detailed in Preparation #5 starting from 2-amino-4-chloro-5-methoxybenzoic acid (CAS RN 181434-76-4, 200 mg; 0.99 mmol; 1.00 eq.) and chloroacetonitrile (CAS RN 107-14-2, 189 μL, 2.98 mmol, 3.00 eq.) to give 7-chloro-2-(chloromethyl)-6-methoxy-3H-quinazolin-4-one (202 mg; 79%) as a beige amorphous powder. ¹H NMR (300 MHz, DMSO-d₆) δ=12.67 (br s, 1H), 7.82 (s, 1H), 7.63 (s, 1H), 4.54 (s, 2H), 3.99 (s, 3H). LC/MS (Table 1, Method A) R₁=2.12 min; MS m/z: 259 [M+H]*.

### Step B: 7-chloro-2-[(3,3-dimethylindolin-1-yl)methyl]-6-methoxy-3H-quinazolin-4-one

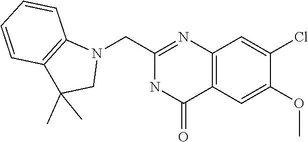

The compound was synthesized using the same procedure detailed in Example #2 starting from 7-chloro-2-(chloromethyl)-6-methoxy-3H-quinazolin-4-one (Example #93, Step A, 198 mg; 0.76 mmol; 1.00 eq.) and 3,3-dimethylindole (CAS RN 1914-02-9, 169 mg; 1.15 mmol; 1.50 eq.). The residue was purified by column chromatography (5 to 40% of EtOAc in cyclohexane) giving 7-chloro-2-[(3,3-dimethylindolin-1-yl)methyl]-6-methoxy-3H-quinazolin-4-one (118 mg; 41%) as a yellow amorphous powder. ¹H NMR (400 MHz, DMSO-d₆) δ=12.40 (s, 1H), 7.75 (s, 1H), 7.62 (s, 1H), 7.02 (dd, J=0.9, 7.3 Hz, 1H), 6.98 (dt, J 1.3, 7.6 Hz, 1H), 6.64 (dt, J=0.9, 7.4 Hz, 1H), 6.60 (d, J=7.8 Hz, 1H), 4.18 (s, 2H), 3.97 (s, 3H), 3.27 (s, 2H), 1.25 (s, 6H). LC/MS (Table 1, Method B) Rₜ=1.85 min; MS m/z: 370 [M+H]⁺.

### Example #94. 2-[(3,3-dimethylindolin-1-yl)methyl]-6,8-dimethoxy-3H-quinazolin-4-one

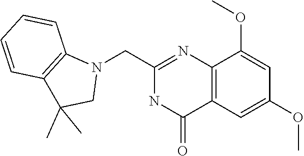

The compound was synthesized using the same procedure detailed in Example #2 starting from 2-(chloromethyl)-6,8-dimethoxy-3H-quinazolin-4-one (CAS RN 1421132-32-2, 200 mg; 0.79 mmol; 1.00 eq.) and 3,3-dimethylindoline (CAS RN 1914-02-9, 173.42 mg; 1.78 mmol; 1.50 eq.). The residue was purified by column chromatography (0 to 100% of EtOAc in cyclohexane) then, by preparative chromatography (Table 2, Condition 1) (column: Kinetex C18. 30×150 mm 5 μm (phenomenex); Flow rate: 42 mL/min; Mobile phase: H₂O+0.1% HCOOH/ACN+0.1% HCOOH; Gradient: 50-65%; run 12 min) giving 2-[(3,3-dimethylindolin-1-yl)methyl]-6,8-dimethoxy-3H-quinazolin-4-one (29 mg; 10%) as a white amorphous powder. ¹H NMR (400 MHz, DMSO-d₆) δ=12.24 (br s, 1H), 7.07 (d, J=2.6 Hz, 1H), 7.02 (dd, J=0.8, 7.2 Hz, 1H), 6.98 (dt, J=1.2, 7.6 Hz, 1H), 6.92 (d, J=2.6 Hz, 1H), 6.64 (dt, J=0.9, 7.4 Hz, 1H), 6.59 (d, J=7.8 Hz, 1H), 4.15 (s, 2H), 3.87 (s, 3H), 3.85 (s, 3H), 3.25 (s, 2H), 1.25 (s, 6H). LC/MS (Table 1, Method B) Rₜ=1.58 min; MS m/z: 366 [M+H]⁺.

### Example #95. 2-[(3,3-dimethylindolin-1-yl)methyl]-4-oxo-3H-quinazoline-6-carbonitrile

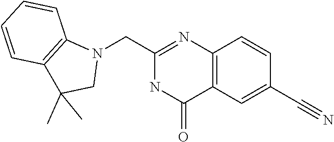

### Step A: 2-(chloromethyl)-4-oxo-3H-quinazoline-6-carbonitrile

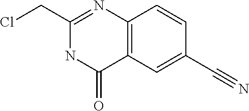

The compound was prepared using the same procedure detailed in Preparation #5 starting from 2-amino-5-cyanobenzoic acid (CAS RN 99767-45-0, 500 mg; 3.08 mmol; 1.00 eq.) and chloroacetonitrile (CAS RN 107-14-2, 587 μL; 9.25 mmol; 3.00 eq.). The reaction mixture was stirred for 48h at rt. The residue was purified by column chromatography (30 to 60% of EtOAc in DCM) giving 2-(chloromethyl)-4-oxo-3H-quinazoline-6-carbonitrile (243 mg; 36%) as a white amorphous powder. ¹H NMR (300 MHz, DMSO-d6) δ=12.98 (br s, 1H), 8.51 (d, J=2.0 Hz, 1H), 8.20 (dd, J=2.1, 8.5 Hz, 1H), 7.82 (d, J=8.6 Hz, 1H), 4.58 (s, 2H). LC/MS (Table 1, Method A) Rₜ=1.78 min; MS m/z: 220 [M+H]⁺.

### Step B: 2-[(3,3-dimethylindolin-1-yl)methyl]-4-oxo-3H-quinazoline-6-carbonitrile

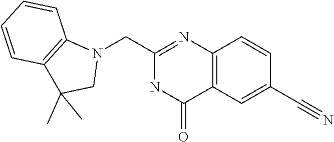

The compound was synthesized using the same procedure detailed in Example #2 starting from 2-(chloromethyl)-4-oxo-3H-quinazoline-6-carbonitrile (Example #95, Step A, 240 mg; 1.09 mmol; 1.00 eq.) and 3,3-dimethylindoline (CAS RN 1914-02-9, 241 mg; 1.64 mmol; 1.50 eq.). The residue was purified by column chromatography (20 to 50% of EtOAc in DCM) giving 2-[(3,3-dimethylindolin-1-yl)methyl]-4-oxo-3H-quinazoline-6-carbonitrile (130 mg; 36%) as a beige amorphous powder. mp=187-197° C. ¹H NMR (400 MHz, DMSO-d₆) δ=12.70 (br s, 1H), 8.49 (d, J=1.8 Hz, 1H), 8.15 (dd, J=2.0, 8.5 Hz, 1H), 7.75 (d, J=8.5 Hz, 1H), 7.03 (dd, J=0.7, 7.3 Hz, 1H) 6.99 (dt, J=1.1, 7.6 Hz, 1H), 6.99 (dt, J=1.2, 7.6 Hz, 1H), 6.65 (t, J=7.6 Hz, 1H), 6.60 (d, J=7.8 Hz, 1H), 4.25 (s, 2H), 3.30 (s, 2H), 1.26 (s, 6H). LC/MS (Table 1, Method B) Rₜ=1.64 min; MS m/z: 331 [M+H]⁺.

### Example #96. 2-[(3,3-dimethylindolin-1-yl)methyl]-6-(3-methoxypropoxy)-3H-quinazolin-4-one

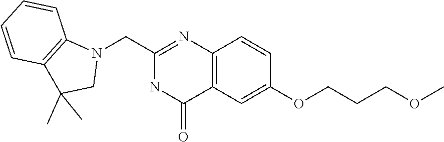

### Step A: 2-(chloromethyl)-6-(3-methoxypropoxy)-3H-quinazolin-4-one

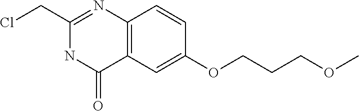

The compound was prepared using the same procedure detailed in Preparation #5 starting from 2-amino-5-(3-methoxypropoxy)benzoic acid (CAS RN 1183040-71-2, 220 mg; 0.98 mmol; 1.00 eq.) and chloroacetonitrile (CAS RN 107-14-2, 186 μL; 2.93 mmol; 3.00 eq.) to give 2-(chloromethyl)-6-(3-methoxypropoxy)-3H-quinazolin-4-one (120 mg; 43%) as a beige amorphous powder. ¹H NMR (DMSO-d₆) δ: 12.52 (br s, 1H), 7.63 (d, J=8.9 Hz, 1H), 7.50 (d, J=2.8 Hz, 1H), 7.40-7.46 (m, 1H), 4.53 (s, 2H), 4.14 (t, J=6.4 Hz, 2H), 3.50 (t, J=6.3 Hz, 2H), 3.26 (s, 3H), 1.99 (quin, J=6.4 Hz, 2H). LC/MS (Table 1, Method A) Rₜ=2.04 min; MS m/z: 283 [M+H]⁺.

### Step B: 2-[(3,3-dimethylindolin-1-yl)methyl]-6-(3-methoxypropoxy)-3H-quinazolin-4-one

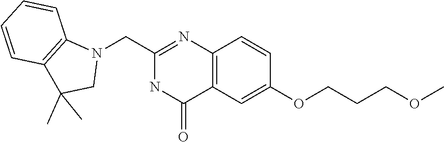

The compound was prepared using the same procedure detailed in Example #2 starting from 2-(chloromethyl)-6-(3-methoxypropoxy)-3H-quinazolin-4-one (Example #96, Step A, 120 mg; 0.42 mmol; 1.00 eq.) in EtOH (3.60 mL), K₂CO₃ (176 mg; 1.27 mmol; 3.00 eq.) and 3,3-dimethylindoline (CAS RN 1914-02-9, 94 mg; 0.64 mmol; 1.50 eq.). The residue was purified by column chromatography (0 to 50% of EtOAc in DCM) giving 2-[(3,3-dimethylindolin-1-yl)methyl]-6-(3-methoxypropoxy)-3H-quinazolin-4-one (90 mg; 54%) as a white powder mp=155-165° C. ¹H NMR (DMSO-d₆) δ: 12.24 (s, 1H), 7.58 (d, J=8.9 Hz, 1H), 7.49 (d, J=2.9 Hz, 1H), 7.39 (dd, J=8.9, 3.0 Hz, 1H), 7.02 (dd, J=7.3, 0.9 Hz, 1H), 6.98 (td, J=7.6, 1.3 Hz, 1H), 6.58-6.68 (m, 2H), 4.17 (s, 2H), 4.13 (t, J=6.5 Hz, 2H), 3.49 (t, J=6.3 Hz, 2H), 3.27 (s, 2H), 3.26 (s, 3H), 1.99 (quin, J=6.4 Hz, 2H), 1.25 (s, 6H). LC/MS (Table 1, Method B) Rₜ=1.74 min; MS m/z: 394 [M+H]⁺.

### Example #97. 2-[(3,3-dimethylindolin-1-yl)methyl]-6-(2-methoxyethoxy)-3H-quinazolin-4-one

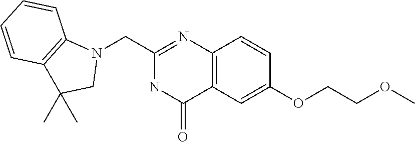 [SMILES: COCCOc1ccc2nc(CN3CC(C)(C)c4ccccc43)[nH]c(=O)c2c1]

### Step A: 2-(chloromethyl)-6-(2-methoxyethoxy)-3H-quinazolin-4-one

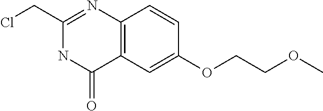 [SMILES: COCCOc1ccc2nc(CCl)[nH]c(=O)c2c1]

The compound was prepared using the same procedure detailed in Preparation #5 starting from 2-amino-5-(2-methoxyethoxy)benzoic acid (CAS RN 783256-55-3, 115 mg, 0.52 mmol, 1.00 eq.) and chloroacetonitrile (CAS RN 107-14-2, 100 μL; 1.57 mmol; 3.00 eq.) to give 2-(chloromethyl)-6-(2-methoxyethoxy)-3H-quinazolin-4-one (50 mg; 36%) as a beige amorphous powder. ¹H NMR (300 MHz, DMSO-d₆) δ=12.51 (br s, 1H), 7.59 (d, J=8.9 Hz, 1H), 7.50 (d, J=3.0 Hz, 1H), 7.44-7.37 (m, 1H), 4.51 (s, 2H), 4.24-4.17 (m, 2H), 3.73-3.67 (m, 2H), 3.32 (s, 3H).

### Step B: 2-[(3,3-dimethylindolin-1-yl)methyl]-6-(2-methoxyethoxy)-3H-quinazolin-4-one

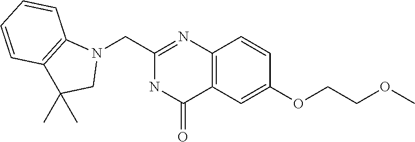 [SMILES: COCCOc1ccc2nc(CN3CC(C)(C)c4ccccc43)[nH]c(=O)c2c1]

The compound was prepared using the same procedure detailed in Example #2 starting from 2-(chloromethyl)-6-(2-methoxyethoxy)-3H-quinazolin-4-one (Example #97, Step A, 50 mg; 0.19 mmol; 1.00 eq.) and 3,3-dimethylindoline (CAS RN 1914-02-9, 41.1 mg; 0.28 mmol; 1.50 eq.). The residue was purified by column chromatography (5 to 40% of EtOAc in cyclohexane) giving 2-[(3,3-dimethylindolin-1-yl)methyl]-6-(2-methoxyethoxy)-3H-quinazolin-4-one (34 mg; 48%) as a beige amorphous powder. ¹H NMR (400 MHz, DMSO-d₆) δ=12.25 (s, 1H), 7.58 (d, J=8.9 Hz, 1H), 7.50 (d, J=3.0 Hz, 1H), 7.41 (dd, J=3.0, 8.9 Hz, 1H), 7.02 (dd, J=0.9, 7.2 Hz, 1H), 6.98 (dt, J=1.2, 7.6 Hz, 1H), 6.67-6.59 (m, 2H), 4.23-4.18 (m, 2H), 4.17 (s, 2H), 3.72-3.68 (m, 2H), 3.32 (s, 3H), 3.27 (s, 2H), 1.25 (s, 6H). LC/MS (Table 1, Method B) Rₜ=1.63 min; MS m/z: 380 [M+H]⁺.

### Example #99. 6-cyclopropyl-2-(indolin-1-ylmethyl)-3H-quinazolin-4-one

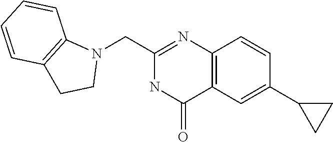 [SMILES: O=c1[nH]c(CN2CCc3ccccc32)nc2ccc(C3CC3)cc12]

6-bromo-2-(indolin-1-ylmethyl)-3H-quinazolin-4-one (Preparation #6, 250 mg; 0.7 mmol; 1.00 eq.) and potassium cyclopropyltrifluoroborate (CAS RN 1065010-87-8, 151 mg; 1.02 mmol; 1.50 eq.) were dissolved in toluene (4.4 mL) and H₂O (0.5 mL). The mixture was degassed with argon bubbling. K₃PO₄ (434 mg; 2.04 mmol; 3.00 eq.), Pd(OAc)₂ (18.3 mg; 0.08 mmol; 0.12 eq.) and 2-dicyclohexylphosphino-2′,6′-dimethoxy-1,1′-biphenyl (67.1 mg; 0.16 mmol; 0.24 eq.) were added. The mixture was stirred at 110° C. under N₂ atm, overnight. The mixture was cooled to rt, diluted in H₂O and extracted with EtOAc three times. The combined organic layers were dried over MgSO₄, filtered and concentrated. The residue was purified by column chromatography (0 to 100% of EtOAc in cyclohexane) then purified by preparative chromatography (Table 2, condition 2) (Column: Kinetex C18, 30×150 mm, 5 μm (Phenomenex), flow 42 mL/min, Mobile phase: H₂O/ACN: isocratic 40% of ACN; 30 min) giving 6-cyclopropyl-2-(indolin-1-ylmethyl)-3H-quinazolin-4-one (9 mg; 4%) as a white amorphous powder. ¹H NMR (400 MHz, DMSO-d6) δ=12.17 (br s, 1H), 7.77 (d, J=1.9 Hz, 1H), 7.53 (d, J=8.5 Hz, 1H), 7.50 (dd, J=2.0, 8.5 Hz, 1H), 7.05 (dd, J=0.7, 7.2 Hz, 1H), 6.97 (dt, J=1.2, 7.7 Hz, 1H), 6.60 (dt, J=0.9, 8.0 Hz, 1H) 6.58 (d, J=7.7 Hz, 1H), 4.17 (s, 2H), 3.49 (t, J=8.4 Hz, 2H), 2.93 (t, J=8.3 Hz, 2H), 2.14-2.05 (m, 1H), 1.06-0.99 (m, 2H), 0.78-0.72 (m, 2H). LC/MS (Table 1, Method B) Rₜ=1.62 min; MS m/z: 318 [M+H]⁺.

### Example #100. 6-(azetidin-1-yl)-2-(indolin-1-ylmethyl)-3H-quinazolin-4-one

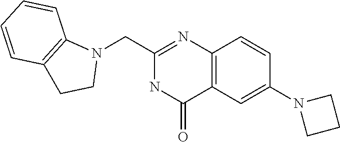 [SMILES: O=c1[nH]c(CN2CCc3ccccc32)nc2ccc(N3CCC3)cc12]

In a microwave tube, 6-bromo-2-(indolin-1-ylmethyl)-3H-quinazolin-4-one (Preparation #6, 250 mg; 0.68 mmol; 1.00 eq.) was dissolved in dry THF (7.3 mL) and the mixture was degassed with Ar bubbling during 10 min. Then azetidine hydrochloride (CAS RN 36520-39-5, 64 mg; 0.68 mmol; 1.00 eq.), tBuOK (229 mg; 2.04 mmol; 3.00 eq.), 2-dicyclohexylphosphino-2′,4′,6′-triisopropylbiphenyl (32 mg; 0.07 mmol; 0.10 eq.) and Pd₂(dba)₃ (62 mg; 0.07 mmol; 0.10 eq.) were successively added under Ar. The mixture was irradiated under microwave for 1 h30 at 120° C. The reaction mixture was concentrated. The residue was purified by column chromatography (0 to 100% of EtOAc in cyclohexane) to give 6-(azetidin-1-yl)-2-(indolin-1-ylmethyl)-3H-quinazolin-4-one (40 mg; 17%) as a yellow amorphous solid. ¹H NMR (400 MHz, DMSO-d₆) δ=11.99 (s, 1H), 7.49 (d, J=8.6 Hz, 1H), 7.05 (d, J=6.7 Hz, 1H), 7.00-6.89 (m, 3H), 6.63-6.56 (m, 2H), 4.12 (s, 2H), 3.90 (t, J=7.2 Hz, 4H), 3.47 (t, J=8.4 Hz, 2H), 2.92 (t, J=8.3 Hz, 2H), 2.34 (quin, J=7.2 Hz, 2H). LC/MS (Table 1, Method B) Rₜ=1.51 min; MS m/z: 333 [M+H]⁺.

### Example #101. 2-(indolin-1-ylmethyl)-6-(3-methoxyazetidin-1-yl)-3H-quinazolin-4-one

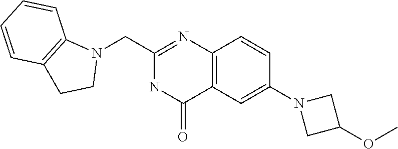 [SMILES: COC1CN(c2ccc3nc(CN4CCc5ccccc54)[nH]c(=O)c3c2)C1]

In a microwave tube, 6-bromo-2-(indolin-1-ylmethyl)-3H-quinazolin-4-one (Preparation #6, 200 mg; 0.54 mmol; 1.00 eq.) was dissolved in dry THE (5.8 mL) and the mixture was degassed with Ar bubbling during 10 min. Then 3-methoxyazetidine hydrochloride (CAS RN 148644-09-1, 67 mg; 0.54 mmol; 1.00 eq.), tBuOK (183 mg; 1.63 mmol; 3.00 eq.), 2-dicyclohexylphosphino-2′,4′,6′-triisopropylbiphenyl (26 mg; 0.05 mmol; 0.10 eq.) and Pd₂(dba)₃ (50 mg; 0.05 mmol; 0.10 eq.) were successively added under Ar. The mixture was irradiated under microwave for 1 h30 at 120° C. The reaction mixture was concentrated. The residue was purified by column chromatography (0 to 100% of EtOAc in cyclohexane) and was further purified by preparative chromatography (Table 2, condition 1) (Column: Kinetex C18, 30×150 mm, 5 m (Phenomenex), flow 42 mL/min, Mobile phase: H₂O with 0.1% of HCOOH/ACN with 0.1% of HCOOH; gradient: 40 to 55% of ACN with 0.1% of HCOOH; run: 16 min) giving 2-(indolin-1-ylmethyl)-6-(3-methoxyazetidin-1-yl)-3H-quinazolin-4-one (15 mg; 8%) as a white amorphous powder after freeze drying. ¹H NMR (400 MHz, DMSO-d₆) δ=12.02 (br s, 1H), 7.50 (d, J=8.6 Hz, 1H), 7.05 (d, J=6.9 Hz, 1H), 7.01-6.93 (m, 3H), 6.63-6.55 (m, 2H), 4.35 (tt, J=4.2, 6.1 Hz, 1H), 4.17-4.13 (m, 2H), 4.13 (s, 2H), 3.70 (dd, J=4.2, 8.5 Hz, 2H), 3.47 (t, J=8.3 Hz, 2H), 3.26 (s, 3H), 2.92 (t, J=8.3 Hz, 2H). LC/MS (Table 1, Method B) Rₜ=1.44 min; MS m/z: 363 [M+H]⁺.

### Example #102. 2-(indolin-1-ylmethyl)-6-pyrrolidin-1-yl-3H-quinazolin-4-one

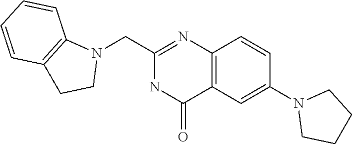 [SMILES: O=c1[nH]c(CN2CCc3ccccc32)nc2ccc(N3CCCC3)cc12]

In a microwave tube, 6-bromo-2-(indolin-1-ylmethyl)-3H-quinazolin-4-one (Preparation #6, 100 mg; 0.28 mmol; 1.00 eq.) was dissolved in dry THF (5.8 mL) and the mixture was degassed with Ar bubbling during 10 min. Then pyrrolidine (CAS RN 123-75-1, 23 μL; 0.28 mmol; 1.00 eq.), tBuOK (47 mg; 0.42 mmol; 1.5 eq.), 2-dicyclohexylphosphino-2′,4′,6′-triisopropylbiphenyl (13.4 mg; 0.03 mmol; 0.10 eq.) and Pd₂(dba)₃ (25.7 mg; 0.03 mmol; 0.10 eq.) were successively added under Ar. The mixture was irradiated under microwave for 1 h at 120° C. The residue was purified by column chromatography (0 to 100% of EtOAc in cyclohexane) giving 2-(indolin-1-ylmethyl)-6-pyrrolidin-1-yl-3H-quinazolin-4-one (58 mg; 54%) as a beige amorphous powder. ¹H NMR (400 MHz, DMSO-d₆) δ=11.93 (s, 1H), 7.50 (d, J=8.8 Hz, 1H), 7.10 (dd, J=2.9, 9.0 Hz, 1H), 7.05 (dd, J=1.0, 7.4 Hz, 1H), 7.01 (d, J=2.9 Hz, 1H), 6.97 (td, J=1.2, 7.7 Hz, 1H), 6.64-6.56 (m, 2H), 4.12 (s, 2H), 3.47 (t, J=8.3 Hz, 2H), 3.31 (br t, J=6.5 Hz, 4H), 2.92 (t, J=8.3 Hz, 2H), 2.05-1.93 (m, 4H). LC/MS (Table 1, Method B) Rₜ=1.64 min; MS m/z: 347 [M+H]⁺.

### Example #103. 2-(indolin-1-ylmethyl)-6-[3-(methoxymethyl)pyrrolidin-1-yl]-3H-quinazolin-4-one

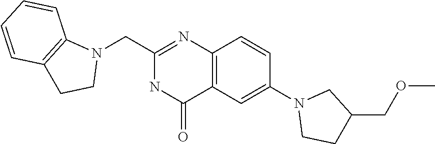 [SMILES: COCC1CCN(c2ccc3nc(CN4CCc5ccccc54)[nH]c(=O)c3c2)C1]

In a microwave tube, 6-bromo-2-(indolin-1-ylmethyl)-3H-quinazolin-4-one (Preparation #6, 400 mg; 1.09 mmol; 1.00 eq.) was dissolved in dry THF (12 mL) and the mixture was degassed with Ar bubbling during 10 min. Then 3-(methoxymethyl)pyrrolidine (CAS RN 936940-38-4, 163.09 mg; 1.42 mmol; 1.30 eq.), tBuOK (183 mg; 1.63 mmol; 1.50 eq.), 2-dicyclohexylphosphino-2′,4′,6′-triisopropylbiphenyl (52 mg; 0.11 mmol; 0.10 eq.) and Pd₂(dba)₃ (100 mg; 0.11 mmol; 0.10 eq.) were successively added under Ar. The mixture was irradiated under microwave for 3h at 120° C. The reaction mixture was concentrated. The residue was purified by column chromatography (0 to 100% of EtOAc in cyclohexane) giving 2-(indolin-1-ylmethyl)-6-[3-(methoxymethyl)pyrrolidin-1-yl]-3H-quinazolin-4-one (154 mg; 36%) as a yellow solid. ¹H NMR (400 MHz, DMSO-d6) δ: 11.94 (s, 1H), 7.50 (d, J=8.9 Hz, 1H), 7.10 (dd, J=9.0, 2.9 Hz, 1H), 7.05 (dd, J=7.4, 1.0 Hz, 1H), 6.95-7.01 (m, 2H), 6.56-6.64 (m, 2H), 4.12 (s, 2H), 3.33-3.52 (m, 7H), 3.28 (s, 3H), 3.08 (dd, J=9.6, 6.6 Hz, 1H), 2.92 (t, J=8.3 Hz, 2H), 2.61 (dt, J=14.3, 7.2 Hz, 1H), 2.10 (dtd, J=12.2, 7.3, 4.6 Hz, 1H), 1.77 (dq, J=12.2, 7.8 Hz, 1H). LC/MS (Table 1, Method B) Rₜ=1.59 min; MS m/z: 391 [M+H]⁺.

### Example #104. 6-[3-(hydroxymethyl)pyrrolidin-1-yl]-2-(indolin-1-ylmethyl)-3H-quinazolin-4-one

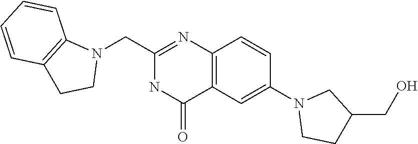 [SMILES: O=c1[nH]c(CN2CCc3ccccc32)nc2ccc(N3CCC(CO)C3)cc12]

The compound was synthesized using the same procedure detailed in Example #103 starting from 6-bromo-2-(indolin-1-ylmethyl)-3H-quinazolin-4-one (Preparation #6, 300 mg; 0.82 mmol; 1.00 eq.) and pyrrolidin-3-ylmethanol (CAS RN 5082-74-6, 107.4 mg; 1.06 mmol; 1.30 eq.). The mixture was irradiated under microwave for 2h at 120° C. The residue was purified by column chromatography (0 to 10% of MeOH in DCM) then a second column chromatography (0 to 50% of EtOAc in DCM) giving 6-[3-(hydroxymethyl)pyrrolidin-1-yl]-2-(indolin-1-ylmethyl)-3H-quinazolin-4-one (49 mg; 15%) as a yellow amorphous powder. ¹H NMR (400 MHz, DMSO-d₆) δ=11.93 (s, 1H), 7.50 (d, J=8.9 Hz, 1H), 7.09 (dd, J=2.9, 9.0 Hz, 1H), 7.05 (dd, J=1.0, 7.3 Hz, 1H), 6.99 (d, J=2.9 Hz, 1H) 6.97 (dt, J=1.0, 7.6 Hz, 1H), 6.60 (dt, J=1.0, 7.5 Hz, 1H) 6.59 (d, J=8.0 Hz, 1H), 4.73 (t, J=5.2 Hz, 1H), 4.12 (s, 2H), 3.52-3.34 (m, 7H), 3.11 (dd, J=6.3, 9.6 Hz, 1H), 2.92 (t, J=8.3 Hz, 2H), 2.49-2.40 (m, 1H), 2.13-2.02 (m, 1H), 1.79 (qd, J=7.4, 12.3 Hz, 1H). LC/MS (Table 1, Method B) Rₜ=1.33 min; MS m/z: 377 [M+H]⁺.

### Example #105. 6-(3-hydroxypyrrolidin-1-yl)-2-(indolin-1-ylmethyl)-3H-quinazolin-4-one

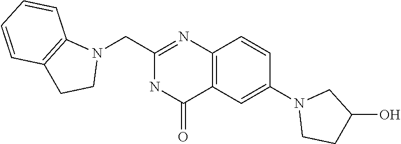 [SMILES: O=c1[nH]c(CN2CCc3ccccc32)nc2ccc(N3CCC(O)C3)cc12]

The compound was synthesized using the same procedure detailed in Example #102 starting from 6-bromo-2-(indolin-1-ylmethyl)-3H-quinazolin-4-one (Preparation #6, 250 mg; 0.68 mmol; 1.00 eq.) and 3-pyrrolidinol (CAS RN 40499-83-0, 55 μL; 0.68 mmol; 1.00 eq.). The mixture was irradiated under microwave for 1 h30 at 120° C. The residue was purified by column chromatography (0 to 10% of MeOH in DCM) giving 6-(3-hydroxypyrrolidin-1-yl)-2-(indolin-1-ylmethyl)-3H-quinazolin-4-one (54 mg; 21%) as brown oil. ¹H NMR (400 MHz, DMSO-d₆) δ=11.94 (s, 1H), 7.50 (d, J=8.9 Hz, 1H), 7.09 (dd, J=3.0, 8.9 Hz, 1H), 7.05 (dd, J=0.9, 7.3 Hz, 1H), 7.01-6.94 (m, 2H), 6.63-6.57 (m, 2H), 5.00 (d, J=3.7 Hz, 1H), 4.47-4.40 (m, 1H), 4.12 (s, 2H), 3.53-3.33 (m, 5H), 3.15 (dd, J=1.0, 10.5 Hz, 1H), 2.92 (t, J=8.3 Hz, 2H), 2.14-2.01 (m, 1H), 1.98-1.89 (m, 1H). LC/MS (Table 1, Method B) Rₜ=1.27 min; MS m/z: 363 [M+H]⁺.

### Example #106. 2-(indolin-1-ylmethyl)-6-(1-piperidyl)-3H-quinazolin-4-one

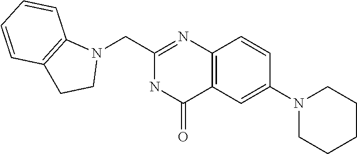 [SMILES: O=c1[nH]c(CN2CCc3ccccc32)nc2ccc(N3CCCCC3)cc12]

The compound was synthesized using the same procedure detailed in Example #102 starting from 6-bromo-2-(indolin-1-ylmethyl)-3H-quinazolin-4-one (Preparation #6, 300 mg; 0.82 mmol; 1.00 eq.) and piperidine (CAS RN 110-89-4, 81 μL; 0.82 mmol; 1.00 eq.). The mixture was irradiated under microwave for 1 h30 at 120° C. The residue was purified by column chromatography (0 to 100% of EtOAc in cyclohexane) giving 2-(indolin-1-ylmethyl)-6-(I-piperidyl)-3H-quinazolin-4-one (76 mg; 24%) as a beige amorphous powder. ¹H NMR (400 MHz, DMSO-d₆) δ=12.03 (s, 1H), 7.53-7.47 (m, 2H), 7.39 (dd, J=1.2, 2.0 Hz, 1H), 7.05 (d, J=6.6 Hz, 1H), 6.97 (dt, J=1.2, 7.7 Hz, 1H), 6.63-6.57 (m, 2H), 4.13 (s, 2H), 3.47 (t, J=8.4 Hz, 2H), 3.27-3.21 (m, 4H), 2.92 (t, J=8.2 Hz, 2H), 1.68-1.52 (m, 6H). LC/MS (Table 1, Method B) R^{t}=1.66 min; MS m/z: 361 [M+H]⁺.

### Example #119. 6-[(3,3-dimethylindolin-1-yl)methyl]-7H-[1,3]dioxolo[4,5-g]quinazolin-8-one

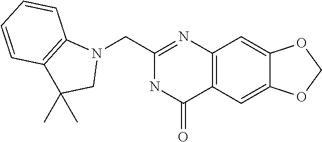 [SMILES: CC1(C)CN(Cc2nc3cc4c(cc3c(=O)[nH]2)OCO4)c2ccccc21]

A mixture of 6-(chloromethyl)-7H-[1,3]dioxolo[4,5-g]quinazolin-8-one (CAS RN 1691758-45-8, 150 mg; 0.63 mmol; 1.00 eq.), 3,3-dimethylindoline (CAS RN 1914-02-9, 138.81 mg; 0.94 mmol; 1.50 eq.) and K₂CO₃ (260 mg; 1.89 mmol; 3 eq.) in EtOH (3.75 mL) was stirred at 80° C. overnight. Reaction mixture was concentrated to dryness. The residue was purified by column chromatography (0 to 40% of EtOAc in DCM) providing 6-[(3,3-dimethylindolin-1-yl)methyl]-7H-[1,3]dioxolo[4,5-g]quinazolin-8-one (96 mg; 44%) as a yellow amorphous powder. mp=199-221° C. ¹H NMR (400 MHz, DMSO-d₆) δ=12.23 (br s, 1H), 7.40 (s, 1H), 7.09 (s, 1H), 7.02 (dd, J=0.8, 7.2 Hz, 1H), 6.98 (dt, J=1.3, 7.6 Hz, 1H), 6.64 (dt, J=0.9, 7.3 Hz, 1H), 6.60 (d, J=7.7 Hz, 1H), 6.19 (s, 2H), 4.15 (s, 2H), 3.26 (s, 2H), 1.25 (s, 6H). LC/MS (Table 1, Method B) Rₜ=1.60 min; MS m/z: 350 [M+H]⁺.

### Example #120. 6-chloro-2-(1-indolin-1-ylethyl)-3H-quinazolin-4-one

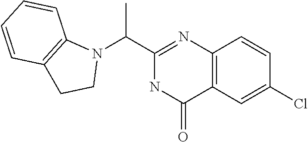 [SMILES: CC(c1nc2ccc(Cl)cc2c(=O)[nH]1)N1CCc2ccccc21]

The compound was synthesized using the same procedure detailed in Example #1 starting from 6-chloro-2-(1-chloroethyl)-3H-quinazolin-4-one (CAS RN 2090878-67-2, 261 mg; 1.07 mmol; 1.00 eq.), indoline (CAS RN 496-15-1, 0.13 mL; 1.18 mmol; 1.10 eq.) providing 6-chloro-2-(1-indolin-1-ylethyl)-3H-quinazolin-4-one (120 mg; 34%) as a white amorphous powder. ¹H NMR (400 MHz, DMSO-d₆) δ=12.44 (br s, 1H), 8.04 (d, J=2.4 Hz, 1H), 7.83 (dd, J=2.5, 8.7 Hz, 1H), 7.69 (d, J=8.7 Hz, 1H), 7.03 (dd, J=0.8, 7.2 Hz, 1H), 6.93 (dt, J=1.1, 7.6 Hz, 1H), 6.56 (dt, J=0.8, 7.3 Hz, 1H), 6.44 (d, J=7.7 Hz, 1H), 4.62 (q, J=6.9 Hz, 1H), 3.61 (q, J=8.2 Hz, 1H), 3.51 (q, J=8.5 Hz, 1H), 2.91 (t, J=8.4 Hz, 2H), 1.49 (d, J=6.9 Hz, 3H). LC/MS (Table 1, Method B) Rₜ=1.78 min; MS m/z: 326 [M+H]⁺.

### Example #122. 2-(3,4-dihydro-1H-isoquinolin-2-ylmethyl)-6-methoxy-3H-quinazolin-4-one

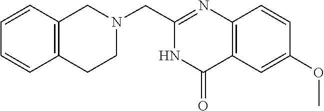 [SMILES: COc1ccc2nc(CN3CCc4ccccc4C3)[nH]c(=O)c2c1]

The compound was synthesized using the same procedure detailed in Example #2 starting from 2-(chloromethyl)-6-methoxy-3H-quinazolin-4-one (CAS RN 1263413-60-0, 200 mg; 0.89 mmol; 1.00 eq.), 1,2,3,4-tetrahydroisoquinoline (CAS RN 91-21-4, 168 μL; 1.34 mmol; 1.50 eq.) and K₂CO₃ (369 mg; 2.67 mmol; 3.00 eq.) in EtOH (4 mL) was stirred at 80° C. overnight and the reaction was further stirred at room temperature for 24 h. The reaction mixture was filtered through a sintered filter funnel, washed with MeOH. The filtrate was concentrated under reduced pressure and the residue was washed with ACN and filtered through a sintered filter funnel. The residue was dissolved in water then the aqueous layer was extracted with a large quantity of DCM. The organic layer was dried over MgSO₄, filtered and concentrated under reduced pressure to provide 2-(3,4-dihydro-1H-isoquinolin-2-ylmethyl)-6-methoxy-3H-quinazolin-4-one (172 mg; 60%) as a white amorphous powder. mp=174-179° C. ¹H NMR (400 MHz, DMSO-d6)=11.88-12.01 (m, 1H), 7.63 (d, J=8.91 Hz, 1H), 7.50 (d, J=2.97 Hz, 1H), 7.41 (dd, J=2.97, 8.91 Hz, 1H), 7.07-7.16 (m, 3H), 7.00-7.06 (m, 1H), 3.87 (s, 3H), 3.68 (s, 2H), 3.60 (s, 2H), 2.82 (dd, J=4.29, 9.57 Hz, 4H). LC/MS (Table 1, Method B) Rₜ=0.98 min; MS m/z: 322 [M+H]⁺.

### Example #123. 2-[(4-hydroxy-3,4-dihydro-1H-isoquinolin-2-yl)methyl]-6-methoxy-3H-quinazolin-4-one

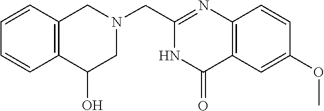 [SMILES: COc1ccc2nc(CN3Cc4ccccc4C(O)C3)[nH]c(=O)c2c1]

The compound was synthesized using the same procedure detailed in Example #2 starting from 2-(chloromethyl)-6-methoxy-3H-quinazolin-4-one (CAS RN 1263413-60-0, 150 mg; 0.67 mmol; 1.00 eq.), 1,2,3,4-tetrahydro-4-isoquinolinol HCl (CAS RN 13691-36-6, 161 mg; 0.87 mmol; 1.30 eq.) and K₂CO₃ (277 mg; 2.00 mmol; 3.00 eq.) in EtOH (3 mL). The reaction was stirred at 70° C. overnight. 20 mL of water were added and the solution was acidified until pH 6 then filtered. The solid was washed with water (3×) then Et₂O (3×). The solid was dried under reduce pressure at rt for 2h to give the expected 2-[(4-hydroxy-3,4-dihydro-1H-isoquinolin-2-yl)methyl]-6-methoxy-3H-quinazolin-4-one (149 mg; 63%) as a brown solid. ¹H NMR (400 MHz, DMSO-d6) δ 11.91 (br s, 1H), 7.62 (d, J=8.80 Hz, 1H), 7.50 (d, J=2.86 Hz, 1H), 7.38-7.45 (m, 2H), 7.14-7.26 (m, 2H), 7.06 (d, J=7.26 Hz, 1H), 5.43 (d, J=8.80 Hz, 1H), 4.57 (td, J=4.44, 8.61 Hz, 1H), 3.87 (s, 3H), 3.49-3.81 (m, 4H), 2.92 (dd, J=3.74, 11.33 Hz, 1H), 2.78 (dd, J=4.95, 11.33 Hz, 1H). LC/MS (Table 1, Method B) Rₜ=1.07 min; MS m/z: 336 [M−H]⁻.

### Example #124. 2-[(5-fluoro-3,4-dihydro-1H-isoquinolin-2-yl)methyl]-6-methoxy-3H-quinazolin-4-one

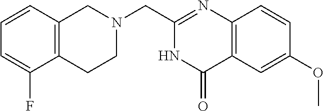 [SMILES: COc1ccc2nc(CN3CCc4c(F)cccc4C3)[nH]c(=O)c2c1]

The compound was synthesized using the same procedure detailed in Example #1 starting from 2-(chloromethyl)-6-methoxy-3H-quinazolin-4-one (CAS RN 1263413-60-0, 140 mg; 0.62 mmol; 1.00 eq.), 5-Fluoro-1,2,3,4-tetrahydro-4-isoquinoline (CAS RN 406923-64-6, 122 mg; 0.81 mmol; 1.30 eq.) and K₂CO₃ (129 mg; 0.93 mmol; 1.50 eq.) in EtOH (2.8 mL). The reaction was stirred at 70° C. overnight. 20 mL of water were added and the solution was acidified until pH 6 then filtered. The solid was washed with water (3×) then Et₂O (3×). The residue was purified by column chromatography (0 to 50% of EtOAc in DCM) to give the expected 2-[(5-fluoro-3,4-dihydro-1H-isoquinolin-2-yl)methyl]-6-methoxy-3H-quinazolin-4-one (89 mg; 42%) as a beige solid. ¹H NMR (400 MHz, DMSO-d6) δ 11.99 (s, 1H), 7.62 (d, J=8.91 Hz, 1H), 7.50 (d, J=2.97 Hz, 1H), 7.41 (dd, J=3.08, 8.91 Hz, 1H), 7.15 (dt, J=5.94, 7.87 Hz, 1H), 6.94-7.04 (m, 1H), 6.92 (d, J=7.48 Hz, 1H), 3.87 (s, 3H), 3.70 (s, 2H), 3.61 (s, 2H), 2.80-2.88 (m, 2H), 2.78 (br d, J=5.28 Hz, 2H). LC/MS (Table 1, Method B) Rₜ=1.20 min; MS m/z: 340 [M+H]⁺.

### Example #125. 6-methoxy-2-[[5-(trifluoromethyl)-3,4-dihydro-1H-isoquinolin-2-yl]methyl]-3H-quinazolin-4-one

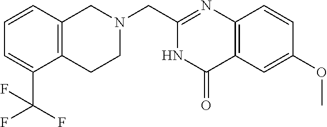 [SMILES: COc1ccc2nc(CN3CCc4c(cccc4C(F)(F)F)C3)[nH]c(=O)c2c1]

The compound was synthesized using the same procedure detailed in Example #2 starting from 2-(chloromethyl)-6-methoxy-3H-quinazolin-4-one (CAS RN 1263413-60-0, 109 mg; 0.49 mmol; 1.00 eq.), 5-(trifluoromethyl)-1,2,3,4-tetrahydroisoquinoline hydrochloride (CAS RN 1074764-69-4, 150 mg; 0.63 mmol; 1.30 eq.) and K₂CO₃ (201 mg; 1.48 mmol; 3.00 eq.) in EtOH (2.3 mL). The reaction was stirred at 70° C. overnight. The crude mixture was concentrated to dryness. The solid was diluted in about 50 mL of water and filtered, then washed twice with about 10 mL of water. Some excess of mass was observed and the compound was diluted in EtOAc and washed twice with water, then brine to obtain 6-methoxy-2-[[5-(trifluoromethyl)-3,4-dihydro-1H-isoquinolin-2-yl]methyl]-3H-quinazolin-4-one (161 mg; 81%) as a white amorphous powder. mp=196-206° C. ¹H NMR (400 MHz, DMSO-d6) δ 11.98 (br s, 1H), 7.62 (d, J=8.91 Hz, 1H), 7.54 (d, J=6.82 Hz, 1H), 7.50 (d, J=2.97 Hz, 1H), 7.36-7.44 (m, 2H), 7.29-7.36 (m, 1H), 3.87 (s, 3H), 3.77 (s, 2H), 3.63 (s, 2H), 2.93-3.04 (m, 2H), 2.82-2.89 (m, 2H). LC/MS (Table 1, Method B) Rₜ=1.48 min; MS m/z: 390 [M+H]⁺.

### Example #126. 2-[(4-fluoroindolin-1-yl)methyl]-6-methoxy-3H-quinazolin-4-one

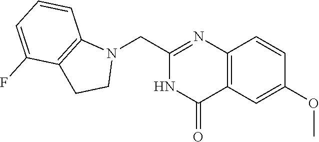 [SMILES: COc1ccc2nc(CN3CCc4c(F)cccc43)[nH]c(=O)c2c1]

The compound was synthesized using the same procedure detailed in Example #2 starting from 2-(chloromethyl)-6-methoxy-3H-quinazolin-4-one (CAS RN 1263413-60-0, 200 mg; 0.89 mmol; 1.00 eq.), 4-fluoro-2,3-dihydro-1H-indole (CAS RN 552866-98-5, 183 mg; 1.34 mmol; 1.50 eq.) and K₂CO₃ (369 mg; 2.67 mmol; 3.00 eq.) in EtOH (5 mL). The reaction was stirred at 80° C. overnight. The crude mixture was concentrated to dryness. The residue was diluted in water and the aqueous phase was extracted by EtOAc. The organic layer was dried over MgSO₄, filtered, and concentrated to dryness. The crude was purified by column chromatography (0 to 30% of EtOAc in DCM) to provide 2-[(4-fluoroindolin-1-yl)methyl]-6-methoxy-3H-quinazolin-4-one (180 mg; 62%) as a white amorphous powder. mp=222-232° C. ¹H NMR (400 MHz, DMSO-d6) δ 12.25 (br s, 1H), 7.58 (d, J=8.91 Hz, 1H), 7.50 (d, J=2.97 Hz, 1H), 7.40 (dd, J=3.03, 8.86 Hz, 1H), 7.01 (dt, J=6.05, 8.03 Hz, 1H), 6.32-6.49 (m, 2H), 4.23 (s, 2H), 3.86 (s, 3H), 3.61 (t, J=8.47 Hz, 2H), 2.98 (t, J=8.42 Hz, 2H). LC/MS (Table 1, Method B) Rₜ=1.48 min; MS m/z: 326 [M+H]⁺.

### Example #127. 6-chloro-2-[(7-methyl-3,4-dihydro-2H-quinolin-1-yl)methyl]-3H-quinazolin-4-one

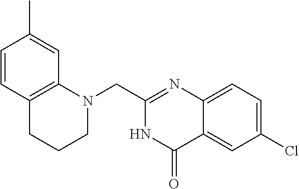 [SMILES: Cc1ccc2c(c1)N(Cc1nc3ccc(Cl)cc3c(=O)[nH]1)CCC2]

The compound was synthesized using the same procedure detailed in Example #1 starting from 2-(chloromethyl)-6-chloro-3H-quinazolin-4-one (CAS RN 2856-54-4, 227 mg; 0.99 mmol; 1.00 eq.) and 7-methyl-1,2,3,4-tetrahydroquinoline (CAS RN 58960-03-5, 160 mg, 1.09 mmol, 1.10 eq.). The residue was purified by column chromatography (0 to 25% of EtOAc in cyclohexane) giving 6-chloro-2-[(7-methyl-3,4-dihydro-2H-quinolin-1-yl)methyl]-3H-quinazolin-4-one (63 mg; 19%) as a white amorphous powder. ¹H NMR (500 MHz, DMSO-d6) δ 12.36 (br s, 1H), 8.03 (d, J=2.5 Hz, 1H), 7.71-7.85 (m, 1H), 7.61 (d, J=8.80 Hz, 1H), 6.78 (d, J=7.5 Hz, 1H), 6.36 (s, 1H), 6.32 (d, J=7.5 Hz, 1H), 4.39 (s, 2H), 3.45-3.50 (m, 2H), 2.68 (t, J=6.3 Hz, 2H), 2.08 (s, 3H), 1.89-1.97 (m, 2H). LC/MS (Table 1, Method B) Rₜ=1.81 min; MS m/z: 340 [M+H]⁺.

### Example #128. 6-chloro-2-[(4-methyl-2,3-dihydroquinoxalin-1-yl)methyl]-3H-quinazolin-4-one

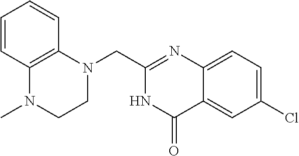 [SMILES: CN1CCN(Cc2nc3ccc(Cl)cc3c(=O)[nH]2)c2ccccc21]

2-(Chloromethyl)-6-chloro-3H-quinazolin-4-one (CAS RN 2856-54-4, 200 mg; 0.87 mmol; 1.00 eq.), 1,2,3,4-tetrahydro-1-methyl-quinoxaline dihydrochloride (CAS RN 1259952-24-3, 289 mg, 1.31 mmol, 1.50 eq.) and K₂CO₃ (543 mg; 3.93 mmol; 4.50 eq.) in EtOH (4 mL) were stirred at 80° C. overnight. The reaction mixture was diluted with water and extracted with EtOAc (3×). The combined organic phases were dried over MgSO₄, filtered and concentrated to dryness to give an orange powder. This residue was purified by column chromatography (0 to 10% of MeOH in DCM) and further purified by preparative chromatography (Table 2, conditions 1) (column: Kinetex C18 30×150 mm 5 μm (phenomenex); Flow rate: 42 mL/min; Mobile phase: H₂O+0.1% HCOOH/ACN+0.1% HCOOH; Gradient: 40-55%; run 20 min) giving 6-chloro-2-[(4- methyl-2,3-dihydroquinoxalin-1-yl)methyl]-3H-quinazolin-4-one (34 mg; 11%) as a beige amorphous powder. ¹H NMR (400 MHz, DMSO-d6) δ 12.39 (br s, 1H), 8.03 (d, J=2.5 Hz, 1H), 7.80 (dd, J=2.5, 8.7 Hz, 1H), 7.63 (d, J=8.7 Hz, 1H), 6.41-6.57 (m, 3H), 6.38 (dd, J=1.5, 8.0 Hz, 1H), 4.35 (s, 2H), 3.59-3.65 (m, 2H), 3.29-3.33 (m, 2H), 2.80 (s, 3H). LC/MS (Table 1, Method B) Rₜ=1.54 min; MS m/z: 341 [M+H]+.

### Example #129. 2-[[3-(2-hydroxyethyl)indolin-1-yl]methyl]-6-methoxy-3H-quinazolin-4-one

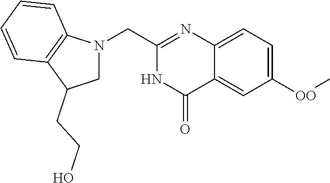

The compound was synthesized using the same procedure detailed in Example #1 starting from 2-(chloromethyl)-6-methoxy-3H-quinazolin-4-one (CAS RN 1263413-60-0, 33 mg; 0.15 mmol; 1.00 eq.), 2,3-dihydro-1H-indole-3-ethanol (CAS RN 40118-09-0, 29 mg; 0.18 mmol; 1.20 eq.) and K₂CO₃ (30.4 mg; 0.22 mmol; 1.50 eq.) in EtOH (3 mL). The reaction was stirred at 80° C. overnight. The reaction mixture was diluted with water and extracted with EtOAc (3×). The combined organic phases dried over MgSO₄, filtered and concentrated to dryness to give as a brown gum. This residue was purified by preparative chromatography (Table 2, conditions 1) (column: Kinetex C18 30×150 mm 5 μm (phenomenex); Flow rate: 42 mL/min; Mobile phase: H₂O+0.1% HCOOH/ACN+0.1% HCOOH; Gradient: 30-45%; run 12 min) giving 2-[[3-(2-hydroxyethyl)indolin-1-yl]methyl]-6-methoxy-3H-quinazolin-4-one (22 ng; 42%) as a white amorphous powder. ¹H NMR (400 MHz, DMSO-d6) δ 12.22 (br s, 1H), 7.60 (d, J=8.8 Hz, 1H), 7.50 (d, J=2.9 Hz, 1H), 7.40 (dd, J=3.0, 8.9 Hz, 1H), 7.05 (d, J=7.3 Hz, 1H), 6.99 (t, J=7.6 Hz, 1H), 6.63 (dt, J=0.8, 7.3 Hz, 1H), 6.60 (d, J=7.8 Hz, 1H), 4.54 (br s, 1H), 4.24 (d, J=15 Hz, 1H), 4.07 (d, J=15 Hz, 1H), 3.87 (s, 3H), 3.67 (t, J=8.6 Hz, 1H), 3.50 (br t, J=5.9 Hz, 2H), 3.22-3.31 (m, 1H), 3.14 (t, J=8.6 Hz, 1H), 1.92-2.05 (m, 1H), 1.56-1.71 (m, 1H). LC/MS (Table 1, Method B) Rₜ=1.25 min; MS m/z: 352 [M+H]⁺.

### Example #130. 6-chloro-2-[(8-methoxy-3,4-dihydro-1H-isoquinolin-2-yl)methyl]-3H-quinazolin-4-one

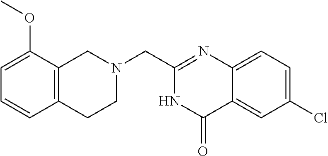 [SMILES: COc1cccc2c1CN(Cc1nc3ccc(Cl)cc3c(=O)[nH]1)CC2]

The compound was synthesized using the same procedure detailed in Example #2 starting from 2-(chloromethyl)-6-chloro-3H-quinazolin-4-one (CAS RN 2856-54-4, 200 mg; 0.87 mmol; 1.00 eq.), 1,2,3,4-tetrahydro-8-methoxy-isoquinoline (CAS RN 34146-68-4, 214 mg, 1.31 mmol, 1.50 eq.). The residue was purified by column chromatography (5 to 40% of EtOAc in cyclohexane) giving 6-chloro-2-[(8-methoxy-3,4-dihydro-1H-isoquinolin-2-yl)methyl]-3H-quinazolin-4-one (135 mg; 43 15%) as a white amorphous powder. mp=195-203° C. ¹H NMR (400 MHz, DMSO-d₆) δ 12.18 (br s, 1H), 8.05 (d, J=2.5 Hz, 1H), 7.84 (dd, J=2.5, 8.7 Hz, 1H), 7.71 (d, J=8.7 Hz, 1H), 7.11 (t, J=7.8 Hz, 1H), 6.75 (d, J=7.8 Hz, 1H), 6.71 (d, J=7.7 Hz, 1H), 3.73 (s, 3H), 3.65 (s, 2H), 3.58 (s, 2H), 2.80 (m, 4H). LC/MS (Table 1, Method B) Rₜ=1.23 min; MS m/z: 356 [M+H]⁺.

### Example #131. 2-[1-[(6-methoxy-4-oxo-3H-quinazolin-2-yl)methyl]indolin-3-yl]ethylammonium chloride

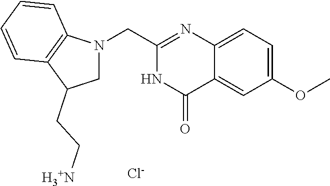

### Step A: tert-butyl N-[2-[1-[(6-methoxy-4-oxo-3H-quinazolin-2-yl)methyl]indolin-3-yl]ethyl]carbamate

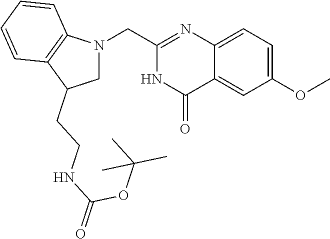 [SMILES: COc1ccc2nc(CN3CC(CCNC(=O)OC(C)(C)C)c4ccccc43)[nH]c(=O)c2c1]

The compound was synthesized using the same procedure detailed in Example #1 starting from 2-(chloromethyl)-6-methoxy-3H-quinazolin-4-one (CAS RN 1263413-60-0, 234 mg; 1.04 mmol; 1.00 eq.), tert-butyl N-(2-indolin-3-ylethyl)carbamate (CAS RN 907969-24-8, 400 mg; 1.36 mmol; 1.30 eq.) and K₂CO₃ (216 mg; 1.57 mmol; 1.50 eq.) in EtOH (5.5 mL). The reaction was stirred at 70° C. overnight. 30 mL of water were added and the solution was acidified until pH 6 then filtered. The solid was washed with water (3×) then Et₂O (3×). The residue was purified by column chromatography (0 to 50% of EtOAc in DCM) giving tert-butyl N-[2-[l1-[(6-methoxy-4-oxo-3H-quinazolin-2-yl)methyl]indolin-3-yl]ethyl]carbamate (198 mg; 42%) as a white amorphous powder. ¹H NMR (400 MHz, DMSO-d6) δ 12.26 (s, 1H), 7.60 (d, J=8.9 Hz, 1H), 7.50 (d, J=2.9 Hz, 1H), 7.40 (dd, J=3.0, 8.9 Hz, 1H), 7.05 (d, J=7.3 Hz, 1H), 7.00 (t, J=7.6 Hz, 1H), 6.90 (t, J=5.7 Hz, 1H), 6.64 (d, J=7.3 Hz, 1H), 6.60 (d, J=8.6 Hz, 1H), 4.26 (d, J=15 Hz, 1H), 4.05 (d, J=15 Hz, 1H), 3.87 (s, 3H), 3.66 (t, J=8.0 Hz, 1H), 3.14-3.24 (m, 1H), 3.08-3.15 (m, 1H), 3.01 (q, J=6.68 Hz, 2H), 1.83-1.97 (m, 1H), 1.53-1.65 (m, 1H), 1.34 (s, 9H). LC/MS (Table 1, Method B) Rₜ=1.64 min; MS m/z: 451 [M+H]+.

### Step B: 2-[l-[(6-methoxy-4-oxo-3H-quinazolin-2-yl)methyl]indolin-3-yl]ethylammonium chloride

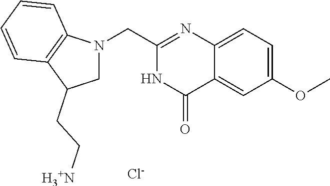

A solution of tert-butyl N-[2-[1-[(6-methoxy-4-oxo-3H-quinazolin-2-yl)methyl]indolin-3-yl]ethyl]carbamate Example #131, step A, (163.00 mg; 0.36 mmol; 1.00 eq.) in hydrochloric acid solution, 4.0 M solution in 1,4-dioxane (1.62 mL; 46.5 mmol) was stirred at rt for 4h. Some more hydrochloric acid solution, 4.0 M solution 1,4-dioxane (0.81 mL; 23.2 mmol) was added and the stirring was maintained at rt overnight affording complete conversion. The reaction mixture was concentrated to give 2-[1-[(6-methoxy-4-oxo-3H-quinazolin-2-yl)methyl]indolin-3-yl]ethylammonium chloride (186 mg; 100%) as pale yellow solid. ¹H NMR (400 MHz, DMSO-d₆) δ 12.6 (br s, 1H), 8.00 (br s, 3H), 7.69 (d, J=8.9 Hz, 1H), 7.52 (d, J=2.8 Hz, 1H), 7.45 (dd, J=2.9, 8.9 Hz, 1H), 7.02-7.9 (m, 2H), 6.66-6.71 (m, 2H), 4.32 (d, J=15 Hz, 1H), 4.21 (d, J=15 Hz, 1H), 3.88 (s, 3H), 3.67 (t, J=8.6 Hz, 1H), 3.28-3.38 (m, 1H), 3.20 (dd, J=6.9, 8.6 Hz, 1H), 2.75-2.94 (m, 2H), 1.99-2.12 (m, 1H), 1.82 (dt, J=4.1, 8.9 Hz, 1H). LC/MS (Table 1, Method B) Rₜ=0.85 min; MS m/z: 351 [M+H]⁺.

### Example #132. 2-(indolin-1-ylmethyl)-6-(4-methoxy-1-piperidyl)-3H-quinazolin-4-one

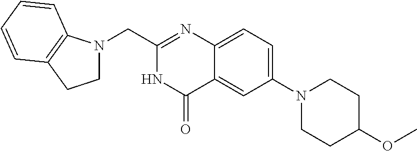 [SMILES: COC1CCN(c2ccc3nc(CN4CCc5ccccc54)[nH]c(=O)c3c2)CC1]

In a microwave tube, 6-bromo-2-(indolin-1-ylmethyl)-3H-quinazolin-4-one (Preparation #6, 85 mg; 0.24 mmol; 1.00 eq.) was dissolved in dry THF (5.5 mL) and the mixture was degassed with Ar bubbling during 10 min. Then 4-methoxypiperidine (CAS RN 4045-24-3, 27.5 mg; 0.24 mmol; 1.00 eq.), tBuOK (40 mg; 0.36 mmol; 1.5 eq.), 2-dicyclohexylphosphino-2′,4′,6′-triisopropylbiphenyl (11.3 mg; 0.02 mmol; 0.10 eq.) and Pd₂(dba)₃ (21.8 mg; 0.02 mmol; 0.10 eq.) were successively added under Ar. The mixture was irradiated under microwave for 30 min at 120° C. The residue was purified by column chromatography (0 to 100% of EtOAc in DCM) giving 2-(indolin-1-ylmethyl)-6-(4-methoxy-1-piperidyl)-3H-quinazolin-4-one (44 mg; 45%) as a yellow amorphous powder. ¹H NMR (400 MHz, DMSO-d₆) δ 12.04 (s, 1H), 7.48-7.56 (m, 2H), 7.40 (d, J=2.4 Hz, 1H), 7.05 (d, J=7.1 Hz, 1H), 6.97 (dt, J=1.1, 7.6 Hz, 1H), 6.52-6.66 (m, 2H), 4.14 (s, 2H), 3.54-3.65 (m, 2H), 3.47 (t, J=8.3 Hz, 2H), 3.34-3.42 (m, 1H), 3.28 (s, 3H), 3.01 (ddd, J=3.1, 9.6, 12.6 Hz, 2H), 2.92 (t, J=8.3 Hz, 2H), 1.90-2.02 (m, 2H), 1.48-1.59 (m, 2H). LC/MS (Table 1, Method B) Rₜ=1.49 min; MS m/z: 391 [M+H]⁺.

### Example #133. 2-[(5-amino-3,4-dihydro-1H-isoquinolin-2-yl)methyl]-6-methoxy-3H-quinazolin-4-one

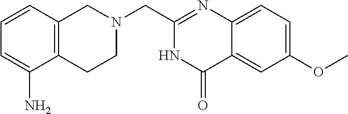 [SMILES: COc1ccc2nc(CN3CCc4c(N)cccc4C3)[nH]c(=O)c2c1]

The compound was synthesized using the same procedure detailed in Example #1 starting from 2-(chloromethyl)-6-methoxy-3H-quinazolin-4-one (CAS RN 1263413-60-0, 100 mg; 0.45 mmol; 1.00 eq.), 1,2,3,4-tetrahydro-5-isoquinolinamine (CAS RN 115955-90-3, 66 mg; 0.45 mmol; 1.00 eq.) and K₂CO₃ (92 mg; 0.67 mmol; 1.50 eq.) in EtOH (2 mL). The reaction was stirred at 80° C. overnight. Reaction media was concentrated to dryness, then diluted with about 30 mL of water. The solid was filtered. Filtrate also containing some expected product was extracted with EtOAc. The organic layer was dried over MgSO₄, filtered, and concentrated to dryness. Both solids were combined and purified by column chromatography (5 to 30% of MeOH in DCM) providing 2-[(5-amino-3,4-dihydro-1H-isoquinolin-2-yl)methyl]-6-methoxy-3H-quinazolin-4-one (60 mg; 40%) as a white amorphous powder. mp=211-233° C. ¹H NMR (400 MHz, DMSO-d₆) δ 11.92 (br s, 1H), 7.62 (d, J=8.9 Hz, 1H), 7.50 (d, J=2.9 Hz, 1H), 7.41 (dd, J=3.0, 8.9 Hz, 1H), 6.80 (t, J=7.6 Hz, 1H), 6.44 (d, J=7.2 Hz, 1H), 6.23 (d, J=7.3 Hz, 1H), 4.79 (br s, 2H), 3.87 (s, 3H), 3.57 (s, 4H), 2.80 (t, J=6.0 Hz, 2H), 2.41-2.54 (m, 2H). LC/MS (Table 1, Method B) Rₜ=0.80 min; MS m/z: 337 [M+H]+.

### Example #138. 7-fluoro-2-(indolin-1-ylmethyl)-3H-quinazolin-4-one

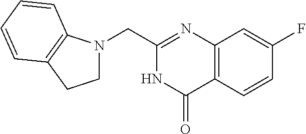 [SMILES: O=c1[nH]c(CN2CCc3ccccc32)nc2cc(F)ccc12]

The compound was synthesized using the same procedure detailed in Example #1 starting from 2-(chloromethyl)-7-Fluoro-3H-quinazolin-4-one (CAS RN 874779-61-0, 94 mg; 0.44 mmol; 1.00 eq.), indoline (CAS RN 496-15-1, 55 μL; 0.49 mmol; 1.10 eq.) and K₂CO₃ (92 mg; 0.66 mmol; 1.50 eq.) in EtOH (1.88 mL). The reaction was stirred at 80° C. overnight. The reaction mixture was diluted with water and the product was extracted with EtOAc (3×). The organic layers were combined, dried over MgSO₄ and concentrated to provide an orange powder. The residue was purified by column chromatography (0 to 40% of EtOAc in cyclohexane) to give the expected 7-fluoro-2-(indolin-1-ylmethyl)-3H-quinazolin-4-one (61 mg; 46%) as an amorphous beige solid. ¹H NMR (400 MHz, DMSO-d₆) δ 12.36 (br s, 1H), 8.17 (dd, J=6.3, 8.8 Hz, 1H), 7.43 (dd, J=2.5, 10.2 Hz, 1H), 7.37 (dt, J=2.5, 8.7 Hz, 1H), 7.06 (d, J=6.71 Hz, 1H), 6.92-7.02 (m, 1H), 6.52-6.68 (m, 2H), 4.20 (s, 2H), 3.50 (t, J=8.3 Hz, 2H), 2.94 (t, J=8.3 Hz, 2H). LC/MS (Table 1, Method B) Rₜ=1.51 min; MS m/z: 296 [M+H]+.

### Example #144. 6-chloro-2-((5-methoxyindolin-1-yl)methyl)quinazolin-4(3H)-one

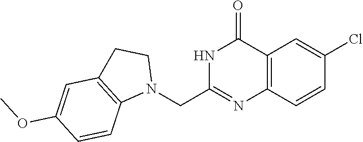 [SMILES: COc1ccc2c(c1)CCN2Cc1nc2ccc(Cl)cc2c(=O)[nH]1]

The Example #144 was synthesized using the same procedure as example #47. 1H NMR (500 MHz, DMSO-d6) δ 12.42 (s, 1H), 8.04 (d, 1H), 7.83 (dd, 1H), 7.68 (d, 1H), 6.75 (d, 1H), 6.58 (dd, 1H), 6.55-6.50 (m, 1H), 4.11 (s, 2H), 3.64 (s, 3H), 3.41 (t, 2H), 2.89 (t, 2H). MS (ES+): 342/344 (M+H)+.

### Example B—Biology

### Material and Method

HMGB1

Construction of pBiT3.1-HMGB1 Plasmid

HMGB1 derived from pCMV6-AC-GFP plasmid (ref. RG205918, Origene) was amplified by PCR using the following set of primers: Seq ID No 1: forward, 5′-GATATCGAATTCATGGGCAAAGGAGATCCTAAGAAGCCGAGAGGC-3′; Seq ID No 2: reverse, 5′-TATCAGCTCGAGACTTCATCATCATCATCTTCTTCTTCATCTTC-3′. The PCR fragment was subsequently digested by EcoRI and XhoI (R3101S and R0146S respectively, New England Biolabs) and inserted between the EcoRI and XhoI sites in the plasmid pBiT3.1-C(ref. N237A, Promega). The resulting plasmid (pBiT3.1-HMGB1) was grown in NEB10-beta competent E. coli (ref. C3019I, New England Biolabs) and purified using the NucleoBond Xtra Midi Kit (ref. 740410, Macherey-Nagel).

Construction of HMGB1 Reporter Cell Line

pBiT3.1-HMGB1 was transfected in MDA-MB-231 cells (ref. ATCC HTB-2) using Lipofectamine 2000 reagent (ref. 11668, Invitrogen). Transfected cells were selected for blasticidin resistance (15 μg/ml, ref. Al 113903 Gibco) in medium containing DMEM 1× (ref. 1995065, Gibco) with 1% penicillin and streptomycin, and 10% fetal bovine serum.

Screen for HMGB1-Releasing Drugs

MDA-MB-231 cells stably expressing HMGB1-HiBiT (10⁴ cells/well) were seeded into white 96-well plates (ref.3917, Corning) and allowed to recover for 24h. Following this, serial 2-fold dilutions of compounds (between 1 μM and 1 nM) were added. Mitoxantrone dihydrochloride (ref. M6545, Sigma-Aldrich) was used as a positive control. Extracellular translocation of HMGB1 was measured using the Nano-Glo HiBiT extracellular detection system (ref. N2422, Promega). Nano-Glo HiBiT Extracellular Reagent was prepared in advance, added to the well and incubated 10 min. Luminescence of the extracellular HMGB1-HiBiT was quantified using the Spark 20M spectrofluorimeter (TECAN)

Calreticulin

Construction of pBiT3.1-Calreticulin Plasmid Calreticulin derived from pCMV6-AC plasmid (ref. SC320287, Origene) was amplified by PCR using the following set of primers: Seq ID No 3: forward, 5′-GATATCGAATTCATGCTGCTATCCGTGCCGCTGCTGCTCGGCCTCCTCG-3′; Seq ID No 4: reverse, 5′-TATCAGCTCGAGACCAGCTCGTCCTTGGCCTGGCCGGGGACATCTTCC-3′. The PCR fragment was subsequently digested by EcoRI and XhoI (R3101S and R0146S respectively, New England Biolabs) and inserted between the EcoRI and XhoI sites in the plasmid pBiT3.1-C(ref. N237A, Promega). The resulting plasmid (pBiT3.1-Calreticulin) was grown in NEB10-beta competent E. coli (ref. C3019I, New England Biolabs) and purified using the NucleoBond Xtra Midi Kit (ref. 740410, Macherey-Nagel).

Construction of Calreticulin Reporter Cell Line

pBiT3.1-Calreticulin was transfected in MDA-MB-231 cells (ref. ATCC HTB-2) using Lipofectamine 2000 reagent (ref. 11668, Invitrogen). Transfected cells were selected for blasticidin resistance (15 μg/ml, ref. Al 113903 Gibco) in medium containing DMEM 1× (ref. 1995065, Gibco) with 1% penicillin and streptomycin, and 10% fetal bovine serum.

Screen for Calreticulin-ER to Plasma Membrane Translocating Drugs

MDA-MB-231 cells stably expressing Calreticulin-HiBiT (104 cells/well) were seeded into white 96-well plates (ref.3917, Corning) and allowed to recover for 24h. Following serial 2-fold dilutions, compounds (between 1 μM and 1 nM) were added. Mitoxantrone dihydrochloride (ref. M6545, Sigma-Aldrich) was used as a positive control. Extracellular translocation of Calreticulin was measured using the Nano-Glo HiBiT extracellular detection system (ref. N2422, Promega). Nano-Glo HiBiT Extracellular Reagent was prepared in advance, added to the well and incubated 10 min.

Luminescence of the extracellular Calreticulin-HiBiT was quantified using the Spark 20M spectrofluorimeter (TECAN).

### RESULTS

The results on Calreticulin release (EC50) and HMGB1 release (High-mobility group box 1) (EC50) are represented in the Table 3 below. Particularly, the results show that the compounds of formula (I) induce calreticulin and HMGB1 release. More particularly, EC50 for Calreticulin release are in μM ranges, preferably lower than 12 μM, and more preferably lower than 1 μM, demonstrating thereby an efficient anticancer effect for the compounds of the present invention.

TABLE 3

| | CalR | HMGB1 | | CalR | HMGB1 | | CalR | HMGB1 |
|---|---|---|---|---|---|---|---|---|
| Compound | EC50M | EC50M | Compound | EC50M | EC50M | Compound | EC50M | EC50M |
| Ex. #99 | 9.75E−08 | 9.75E−08 | Ex. #11 | 1.02E−07 | 1.09E−07 | Ex. #92 | 9.88E−08 | 1.40E−07 |
| Ex. #77 | 1.50E−07 | 1.50E−07 | Ex. #93 | 9.75E−08 | 1.50E−07 | Ex. #2 | 4.00E−07 | 6.00E−07 |
| Ex. #4 | 1.72E−07 | 2.07E−07 | Ex. #74 | 2.00E−07 | 2.10E−07 | Ex. #6 | 2.54E−07 | 2.17E−07 |
| Ex. #80 | 2.20E−07 | 2.25E−07 | Ex. #100 | 2.30E−07 | 2.50E−07 | Ex. #3 | 1.91E−07 | 2.63E−07 |
| Ex. #1 | 2.31E−07 | 2.72E−06 | Ex. #73 | 2.53E−07 | 2.75E−07 | Ex. #81 | 8.00E−08 | 4.10E−07 |
| Ex. #7 | 3.23E−07 | 4.25E−07 | Ex. 22 | 3.39E−07 | 4.29E−07 | Ex. #76 | 2.92E−07 | 5.94E−07 |
| Ex. #38 | 4.99E−07 | 7.21E−07 | Ex. #12 | 5.22E−07 | 7.51E−07 | Ex. #13 | 1.14E−06 | 7.52E−07 |
| Ex. #79 | 7.96E−07 | 8.01E−07 | Ex. #103 | 5.18E−07 | 8.58E−07 | Ex. #36 | 1.80E−06 | 1.01E−06 |
| Ex. #62 | 9.60E−07 | 1.06E−06 | Ex. #43 | 1.07E−06 | 1.15E−06 | Ex. #82 | 1.38E−06 | 1.17E−06 |
| Ex. #40 | 9.41E−07 | 1.26E−06 | Ex. #44 | 1.26E−06 | 1.33E−06 | Ex. #52 | 1.26E−06 | 1.36E−06 |
| Ex. #102 | 5.95E−07 | 1.37E−06 | Ex. #83 | 1.61E−06 | 1.44E−06 | Ex. 23 | 1.96E−06 | 2.00E−06 |
| Ex. #14 | 1.96E−06 | 2.16E−06 | Ex. #53 | 7.30E−07 | 2.51E−06 | Ex. #84 | 6.95E−07 | 2.63E−06 |
| Ex. #16 | 4.90E−07 | 2.66E−06 | Ex. #54 | 1.76E−06 | 3.06E−06 | Ex. #37 | 1.16E−06 | 3.16E−06 |
| Ex. #78 | 2.45E−06 | 3.25E−06 | Ex. #15 | 1.06E−06 | 3.34E−06 | Ex. #5 | 2.31E−06 | 3.46E−06 |
| Ex. #104 | 2.16E−06 | 3.46E−06 | Ex. #42 | 1.76E−06 | 3.55E−06 | Ex. #85 | 1.96E−06 | 3.56E−06 |
| Ex. #86 | 2.56E−06 | 3.86E−06 | Ex. #61 | 4.50E−07 | 3.86E−06 | Ex. #87 | 2.16E−06 | 3.96E−06 |
| Ex. #47 | 2.18E−06 | 3.98E−06 | Ex. #63 | 3.86E−06 | 4.26E−06 | Ex. #45 | 1.21E−06 | 4.66E−06 |
| Ex. #88 | 7.56E−06 | 4.66E−06 | Ex. #58 | 5.24E−07 | 4.81E−06 | Ex. #64 | 2.46E−06 | 5.31E−06 |
| Ex. #46 | 2.71E−06 | 5.51E−06 | Ex. #94 | 3.06E−06 | 5.76E−06 | Ex. #89 | 8.26E−06 | 6.26E−06 |
| Ex. #48 | 5.96E−06 | 6.06E−06 | Ex. #17 | 5.40E−07 | 6.06E−06 | Ex. #119 | 6.16E−06 | 7.56E−06 |
| Ex. #10 | 4.46E−06 | 6.86E−06 | Ex. #59 | 4.41E−06 | 7.51E−06 | Ex. #8 | 5.66E−06 | 8.36E−06 |
| Ex. #97 | 7.36E−06 | 7.56E−06 | Ex. #120 | 4.61E−06 | 7.86E−06 | Ex. #18 | 2.06E−06 | 9.36E−06 |
| Ex. #90 | 5.49E−06 | 8.76E−06 | Ex. #60 | 8.70E−07 | 9.36E−06 | Ex. #19 | 7.36E−06 | 1.02E−05 |
| Ex. #41 | 8.01E−06 | 9.61E−06 | Ex. #91 | 8.11E−06 | 1.01E−05 | Ex. #96 | 9.86E−06 | 1.06E−05 |
| Ex. #101 | 1.00E−05 | 1.02E−05 | Ex. #39 | 8.06E−06 | 1.04E−05 | Ex. #106 | 8.46E−06 | 1.11E−05 |
| Ex. #20 | 4.26E−06 | 1.08E−05 | Ex. #55 | 1.13E−05 | 1.08E−05 | Ex. #95 | 9.96E−06 | 1.15E−05 |
| Ex. #21 | 3.96E−06 | 1.12E−05 | Ex. #49 | 7.56E−06 | 1.12E−05 | Ex. #51 | 1.03E−05 | 1.18E−05 |
| Ex. #50 | 4.66E−06 | 1.16E−05 | Ex. #105 | 9.16E−06 | 1.17E−05 | Ex. #128 | 1.05E−05 | 1.33E−05 |
| Ex. #122 | 1.66E−06 | 1.27E−05 | Ex. #123 | 3.61E−06 | 1.31E−05 | Ex. #124 | 2.16E−06 | 1.35E−05 |
| Ex. #129 | 1.07E−05 | 1.33E−05 | Ex. #127 | 6.96E−06 | 1.34E−05 | Ex. #131 | 1.21E−05 | 1.45E−05 |
| Ex. #133 | 8.16E−06 | 1.40E−05 | Ex. #132 | 1.04E−05 | 1.44E−05 | Ex. #130 | 9.06E−06 | 1.51E−05 |
| Ex. #138 | 1.20E−05 | 1.45E−05 | Ex. #144 | 9.71E−06 | 1.46E−05 | | | |
| Ex. #125 | 9.76E−06 | 1.59E−05 | Ex. #126 | 8.06E−06 | ND | | | |
| Ex. #9 | 1.28E−05 | 1.17E−05 | Ex. #57 | 1.42E−05 | 1.19E−05 | | | |

ND: Not Determined

1-18. (canceled)

19. A compound having the following formula (I): 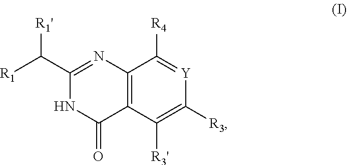
wherein:
R₁ represents a fused aryl heterocycloalkyl selected from the group consisting of:
an indolinyl,
an isoindolinyl,
a 1,2,3,4-tetrahydroquinolinyl,
a 1,2,3,4-tetrahydroisoquinolinyl,
a 2,3-dihydroquinoxalinyl,
a 3,4-dihydro-1,4-benzoxazinyl,
a 2,3,4,5-tetrahydro-1,5-benzodiazepinyl,
a 2,3,4,5-tetrahydro-1-benzazepinyl, and
a 2,3,4,5-tetrahydro-1,5-benzoxazepinyl,
said fused arylheterocycloalkyl is optionally substituted by at least one radical selected from the group consisting of:
a halogen,
a (C₁-C₆)alkyl optionally substituted by at least one hydroxy, an amino or a halogen,
a (C₁-C₆)alkyloxy optionally substituted by at least one hydroxy, an amino or a halogen,
a cyano,
a cycloalkyl or a spirocycloalkyl,
a hydroxy,
a —NH—SO₂(CH₃),
a —NR₅R₆ with R₅ and R₆ being independently a hydrogen, a (C₁-C₆)alkyl or a —COR₈ with R₈ being a (C₁-C₆)alkyl, and
R_{1′} represents a hydrogen or a (C₁-C₆)alkyl;
Y represents:
a nitrogen, or
a —CR₂ group in which R₂ is a radical selected from the group consisting of a hydrogen, a halogen, a (C₁-C₆)alkyl optionally substituted by at least one halogen, and a (C₁-C₆)alkyloxy optionally substituted by at least one halogen;
R₃ and R_{3′} represents independently a radical selected from the group consisting of:
a 3-10 membered ring selected from the group consisting of a cycloalkyl, a heterocycloalkyl, and an aryl, said 3-10 membered ring is optionally substituted by a radical selected from the group consisting of:
a (C₁-C₆)alkyl optionally substituted by a (C₁-C₆)alkyloxy, a hydroxy, a halogen, or a —NR₅R₆ with R₅ and R₆ being independently a hydrogen,
a (C₁-C₆)alkyloxy optionally substituted by a halogen,
a halogen, and
a hydroxy,
a (C₁-C₆)alkyl and a (C₁-C₆)alkyloxy optionally substituted by a halogen, a (C₁-C₆)alkyloxy or a —NH—CO₂-R₇ with R₇ being a (C₁-C₆)alkyl,
a —NR₅R₆ with R₅ and R₆ being independently a hydrogen or a (C₁-C₆)alkyl,
a halogen,
a hydrogen, and
a cyano, or
R₃ may form with R₂ a dioxolanyl; and
R₄ represents a hydrogen or a (C₁-C₆)alkyloxy;
and the stereoisomers, the tautomers, and the pharmaceutical salts thereof;
with the proviso that the compound of formula (I) is not:
6-chloro-2-(indolin-1-ylmethyl)-3H-quinazolin-4-one;
6-chloro-2-(3,4-dihydro-2H-quinolin-1-ylmethyl)-3H-quinazolin-4-one;
6-chloro-2-(3,4-dihydro-1H-isoquinolin-2-ylmethyl)-3H-quinazolin-4-one;
6-chloro-2-[(4-hydroxy-5,8-dimethoxy-3,4-dihydro-1H-isoquinolin-2-yl)methyl]-3H-quinazolin-4-one;
2-(indolin-1-ylmethyl)-6,7-dimethoxy-3H-quinazolin-4-one; and
7-chloro-2-(indolin-1-ylmethyl)-3H-quinazolin-4-one.

20. The compound according to claim 19, wherein Y represents a nitrogen, or a —CR₂ group in which R₂ is a radical selected from the group consisting of a hydrogen, a halogen, a (C₁-C₆)alkyloxy optionally substituted by a halogen, a (C₁-C₆)alkyl optionally substituted by a halogen.

21. The compound according to claim 19, wherein R_{3′} represents a hydrogen.

22. The compound according to claim 19, wherein R₃ represents a radical selected from the group consisting of:
a 3-10 membered ring selected from the group consisting of a cyclopropyl, an azetidinyl, a pyrrolidinyl, a phenyl, and a piperidinyl, said 3-10 membered ring is optionally substituted by a radical selected from the group consisting of:
a methyl substituted by a methoxy or a hydroxy,
a (C₁-C₆)alkyloxy, and
a hydroxy,
a (C₁-C₆)alkyloxy or a (C₁-C₆)alkyloxy substituted by a methoxy or a —O—(CH₂)₄NHCO₂tBu,
a methyl, a trifluoromethyl, or an isopropyl,
a —NR₅R₆ with R₅ and R₆ being a methyl,
a chlorine or a fluorine,
a hydrogen, and
a cyano.

23. The compound according to claim 19, wherein R₃ represents a radical selected from the group consisting of a cyclopropyl, a methoxy, a —NR₅R₆ with R₅ and R₆ being a methyl, an azetidinyl, an isopropyl, a chlorine, a pyrrolidine optionally substituted by a —CH₂—O—CH₃, and a methyl.

24. The compound according to claim 19, wherein R₁ represents an optionally substituted indolinyl or an optionally substituted isoindolinyl.

25. The compound according to claim 24, wherein:
R₁ represents an unsubstituted indolinyl, an indolinyl substituted by at least one radical selected from the group consisting of a halogen, and a (C₁-C₆)alkyl and an indolinyl substituted by two (C₁-C₆)alkyl;
R_{1′} represents a hydrogen;
Y represents:
a nitrogen, or
a —CR₂ group in which R₂ is a radical selected from the group consisting of a hydrogen and a halogen;
R₃ represents a radical selected from the group consisting of:
a cyclopropyl, an azetidinyl, or a pyrrolidinyl optionally substituted by a —CH₂—O—CH₃,
a (C₁-C₆)alkyloxy or a (C₁-C₆)alkyl,
a —NR₅R₆ with R₅ and R₆ being a methyl,
a (C₁-C₆)alkyl, and
a halogen;
R_{3′} represents a hydrogen; and
R₄ represents a hydrogen.

26. The compound according to claim 19, wherein R₁ represents an optionally substituted 1,2,3,4-tetrahydroquinolinyl.

27. The compound according to claim 26, wherein:
R₁ represents an unsubstituted 1,2,3,4-tetrahydroquinolinyl or a 1,2,3,4-tetrahydroquinolinyl substituted by at least one radical selected from the group consisting of a halogen, a (C₁-C₆)alkyl, and a (C₁-C₆)alkyloxy;
R_{1′} represents a hydrogen;
Y represents a —CR₂ group in which R₂ is a hydrogen;
R₃ represents a radical selected from the group consisting of a (C₁-C₆)alkyloxy and a halogen;
R_{3′} represents a hydrogen; and
R₄ represents a hydrogen.

28. The compound according to claim 19, wherein R₁ represents an optionally substituted 1,2,3,4-tetrahydroisoquinolinyl.

29. The compound according to claim 28, wherein:
R₁ represents a 1,2,3,4-tetrahydroisoquinolinyl substituted by a radical selected from the group consisting of a cyano, a hydroxy, a (C₁-C₆)alkyloxy, a (C₁-C₆)alkyl, or a 1,2,3,4-tetrahydroisoquinolinyl substituted by one hydroxy and two (C₁-C₆)alkyloxy;
R_{1′} represents a hydrogen;
Y represents a —CR₂ group in which R₂ is a hydrogen;
R₃ represents a radical selected from the group consisting of a (C₁-C₆)alkyloxy and a halogen;
R_{3′} represents a hydrogen; and
R₄ represents a hydrogen.

30. The compound according to claim 19, wherein R₁ represents an optionally substituted 2,3,4,5-tetrahydro-1,5-benzodiazepinyl or an optionally substituted 2,3,4,5-tetrahydro-1-benzazepinyl.

31. The compound according to claim 30, wherein:
R₁ represents an unsubstituted 2,3,4,5-tetrahydro-1-benzazepinyl, or a 2,3,4,5-tetrahydro-1,5-benzodiazepinyl substituted by a (C₁-C₆)alkyl;
R_{1′} represents a hydrogen;
Y represents a —CR₂ group in which R₂ is a hydrogen;
R₃ represents a radical selected from the group consisting of a (C₁-C₆)alkyloxy and a halogen;
R_{3′} represents a hydrogen; and
R₄ represents a hydrogen.

32. The compound according to claim 19, wherein said compound is selected from the group consisting of:
6-cyclopropyl-2-(indolin-1-ylmethyl)-3H-quinazolin-4-one;
2-[(8-fluoro-3,4-dihydro-2H-quinolin-1-yl)methyl]-6-methoxy-3H-quinazolin-4-one;
6-(dimethylamino)-2-[(3,3-dimethylindolin-1-yl)methyl]-3H-pyrido[3,4-d]pyrimidin-4-one;
2-[(3,3-dimethylindolin-1-yl)methyl]-7-fluoro-6-methoxy-3H-quinazolin-4-one;
7-chloro-2-[(3,3-dimethylindolin-1-yl)methyl]-6-methoxy-3H-quinazolin-4-one;
2-[(3,3-dimethylindolin-1-yl)methyl]-6-methoxy-3H-quinazolin-4-one;
6-methoxy-2-[(3-methylindolin-1-yl)methyl]-3H-quinazolin-4-one, enantiomer 1;
6-(dimethylamino)-2-[(3,3-dimethylindolin-1-yl)methyl]-3H-quinazolin-4-one;
2-[(3-isopropylindolin-1-yl)methyl]-6-methoxy-3H-quinazolin-4-one;
6-(dimethylamino)-7-fluoro-2-(indolin-1-ylmethyl)-3H-quinazolin-4-one;
6-(azetidin-1-yl)-2-(indolin-1-ylmethyl)-3H-quinazolin-4-one;
6-methoxy-2-[(3-methylindolin-1-yl)methyl]-3H-quinazolin-4-one;
2-(indolin-1-ylmethyl)-6-methoxy-3H-quinazolin-4-one;
6-(dimethylamino)-2-(indolin-1-ylmethyl)-3H-quinazolin-4-one;
2-(indolin-1-ylmethyl)-6-isopropyl-3H-quinazolin-4-one;
2-[(6-fluoroindolin-1-yl)methyl]-6-methoxy-3H-quinazolin-4-one;
6-methoxy-2-(2,3,4,5-tetrahydro-1-benzazepin-1-ylmethyl)-3H-quinazolin-4-one;
7-fluoro-2-(indolin-1-ylmethyl)-6-methoxy-3H-quinazolin-4-one;
6-chloro-2-[(3-methylindolin-1-yl)methyl]-3H-quinazolin-4-one enantiomer 1;
2-[(8-chloro-3,4-dihydro-2H-quinolin-1-yl)methyl]-6-methoxy-3H-quinazolin-4-one;
6-methoxy-2-[(8-methoxy-3,4-dihydro-2H-quinolin-1-yl)methyl]-3H-quinazolin-4-one;
2-[(3,3-dimethylindolin-1-yl)methyl]-6-methoxy-3H-pyrido[3,4-d]pyrimidin-4-one;
2-(indolin-1-ylmethyl)-6-[3-(methoxymethyl)pyrrolidin-1-yl]-3H-quinazolin-4-one;
6-chloro-2-(2,3,4,5-tetrahydro-1-benzazepin-1-ylmethyl)-3H-quinazolin-4-one;
6-chloro-2-[(4-methylindolin-1-yl)methyl]-3H-quinazolin-4-one;
2-(indolin-1-ylmethyl)-6-(trifluoromethoxy)-3H-quinazolin-4-one;
6-chloro-2-[(3,3-dimethylindolin-1-yl)methyl]-3H-quinazolin-4-one;
6-chloro-2-[(7-methylindolin-1-yl)methyl]-3H-quinazolin-4-one;
6-chloro-2-[(5-methoxy-3,4-dihydro-2H-quinolin-1-yl)methyl]-3H-quinazolin-4-one;
2-(indolin-1-ylmethyl)-6-pyrrolidin-1-yl-3H-quinazolin-4-one;
6-methoxy-2-[(1-methyl-3,4-dihydro-2H-1,5-benzodiazepin-5-yl)methyl]-3H-quinazolin-4-one;
6-methoxy-2-[(5-methoxy-2,3-dihydro-1,4-benzoxazin-4-yl)methyl]-3H-quinazolin-4-one;
6-chloro-2-[(5-methyl-3,4-dihydro-2H-quinolin-1-yl)methyl]-3H-quinazolin-4-one;
2-(indolin-1-ylmethyl)-6-methyl-3H-quinazolin-4-one;
2-[(6-methoxy-4-oxo-3H-quinazolin-2-yl)methyl]-3,4-dihydro-1H-isoquinoline-5-carbonitrile;
6-chloro-2-[(8-methoxy-3,4-dihydro-2H-quinolin-1-yl)methyl]-3H-quinazolin-4-one;
6-chloro-2-[(3-methylindolin-1-yl)methyl]-3H-quinazolin-4-one;
2-(indolin-1-ylmethyl)-6-methoxy-3H-pyrido[3,4-d]pyrimidin-4-one;
2-(3,4-dihydro-2H-quinolin-1-ylmethyl)-6-methoxy-3H-quinazolin-4-one;
6-methoxy-2-[(3-methylindolin-1-yl)methyl]-3H-quinazolin-4-one, enantiomer 2;
6-[3-(hydroxymethyl)pyrrolidin-1-yl]-2-(indolin-1-ylmethyl)-3H-quinazolin-4-one;
6-chloro-2-(spiro[cyclopentane-1,3′-indoline]-1′-ylmethyl)-3H-quinazolin-4-one;
2-(indolin-1-ylmethyl)-6-(trifluoromethyl)-3H-quinazolin-4-one;
2-(indolin-1-ylmethyl)-7-methoxy-3H-quinazolin-4-one;
2-(indolin-1-ylmethyl)-6-isopropoxy-3H-quinazolin-4-one;
6-chloro-2-[(5-fluoroindolin-1-yl)methyl]-3H-quinazolin-4-one;
6-chloro-2-[(1-methyl-3,4-dihydro-2H-1,5-benzodiazepin-5-yl)methyl]-3H-quinazolin-4-one;
6-chloro-2-[(6-methylindolin-1-yl)methyl]-3H-quinazolin-4-one;
6-fluoro-2-(indolin-1-ylmethyl)-3H-quinazolin-4-one;
6-chloro-2-[(5-methoxy-3,4-dihydro-1H-isoquinolin-2-yl)methyl]-3H-quinazolin-4-one;
6-chloro-2-(1,2,3,4-tetrahydroisoquinolin-2-ium-2-ylmethyl)-3H-quinazolin-4-one chloride;
6-chloro-2-(3,4-dihydro-2H-1,5-benzoxazepin-5-ylmethyl)-3H-quinazolin-4-one;
6-chloro-2-[(5-methylindolin-1-yl)methyl]-3H-quinazolin-4-one;
2-[(3,3-dimethylindolin-1-yl)methyl]-6,8-dimethoxy-3H-quinazolin-4-one;
2-[(5-bromoindolin-1-yl)methyl]-6-chloro-3H-quinazolin-4-one;
6-methoxy-2-[(5-methoxy-3,4-dihydro-1H-isoquinolin-2-yl)methyl]-3H-quinazolin-4-one;
2-(isoindolin-2-ylmethyl)-6-methoxy-3H-quinazolin-4-one;
6-[(3,3-dimethylindolin-1-yl)methyl]-7H-[1,3]dioxolo[4,5-g]quinazolin-8-one;
2-[(3,3-dimethylindolin-1-yl)methyl]-6-(2-methoxyethoxy)-3H-quinazolin-4-one;
6-chloro-2-(1-indolin-1-ylethyl)-3H-quinazolin-4-one;
2-[(7-fluoroindolin-1-yl)methyl]-6-methoxy-3H-quinazolin-4-one;
tert-butyl N-[4-[[2-(indolin-1-ylmethyl)-4-oxo-3H-quinazolin-6-yl]oxy]butyl]carbamate;
6-chloro-2-[(7-methyl-3,4-dihydro-1H-isoquinolin-2-yl)methyl]-3H-quinazolin-4-one;
2-[(5-bromo-3,4-dihydro-1H-isoquinolin-2-yl)methyl]-6-methoxy-3H-quinazolin-4-one;
6-chloro-2-[(2-methylindolin-1-yl)methyl]-3H-quinazolin-4-one;
2-(indolin-1-ylmethyl)-7-(trifluoromethyl)-3H-quinazolin-4-one;
6-methoxy-2-[(8-methoxy-3,4-dihydro-1H-isoquinolin-2-yl)methyl]-3H-quinazolin-4-one;
2-(indolin-1-ylmethyl)-6-(3-methoxyazetidin-1-yl)-3H-quinazolin-4-one;
6-chloro-2-[(3-methylindolin-1-yl)methyl]-3H-quinazolin-4-one enantiomer 2;
2-[(3,3-dimethylindolin-1-yl)methyl]-6-(3-methoxypropoxy)-3H-quinazolin-4-one;
2-[(5-chloro-3,4-dihydro-1H-isoquinolin-2-yl)methyl]-6-methoxy-3H-quinazolin-4-one;
6-chloro-2-[(7-methoxy-3,4-dihydro-2H-quinolin-1-yl)methyl]-3H-quinazolin-4-one;
2-(indolin-1-ylmethyl)-6-(1-piperidyl)-3H-quinazolin-4-one;
N-[2-[(6-methoxy-4-oxo-3H-quinazolin-2-yl)methyl]-3,4-dihydro-1H-isoquinolin-5-yl]methanesulfonamide;
6-chloro-2-[(7-methoxyindolin-1-yl)methyl]-3H-quinazolin-4-one;
2-[(3,3-dimethylindolin-1-yl)methyl]-4-oxo-3H-quinazoline-6-carbonitrile;
6-chloro-2-(isoindolin-2-ylmethyl)-3H-quinazolin-4-one;
6-(3-hydroxypyrrolidin-1-yl)-2-(indolin-1-ylmethyl)-3H-quinazolin-4-one;
2-(3,4-dihydro-1H-isoquinolin-2-ylmethyl)-6-methoxy-3H-quinazolin-4-one;
2-[(4-hydroxy-3,4-dihydro-1H-isoquinolin-2-yl)methyl]-6-methoxy-3H-quinazolin-4-one;
6-chloro-2-[(4-methyl-2,3-dihydroquinoxalin-1-yl)methyl]-3H-quinazolin-4-one;
2-[[3-(2-hydroxyethyl)indolin-1-yl]methyl]-6-methoxy-3H-quinazolin-4-one;
6-chloro-2-[(7-methyl-3,4-dihydro-2H-quinolin-1-yl)methyl]-3H-quinazolin-4-one;
2-[(5-fluoro-3,4-dihydro-1H-isoquinolin-2-yl)methyl]-6-methoxy-3H-quinazolin-4-one;
2-[(5-amino-3,4-dihydro-1H-isoquinolin-2-yl)methyl]-6-methoxy-3H-quinazolin-4-one;
2-(indolin-1-ylmethyl)-6-(4-methoxy-1-piperidyl)-3H-quinazolin-4-one;
2-[1-[(6-methoxy-4-oxo-3H-quinazolin-2-yl)methyl]indolin-3-yl]ethylammonium chloride;
7-fluoro-2-(indolin-1-ylmethyl)-3H-quinazolin-4-one;
6-chloro-2-((5-methoxyindolin-1-yl)methyl)quinazolin-4(3H)-one;
6-chloro-2-[(8-methoxy-3,4-dihydro-1H-isoquinolin-2-yl)methyl]-3H-quinazolin-4-one;
6-methoxy-2-[[5-(trifluoromethyl)-3,4-dihydro-1H-isoquinolin-2-yl]methyl]-3H-quinazolin-4-one;
2-[(4-fluoroindolin-1-yl)methyl]-6-methoxy-3H-quinazolin-4-one;
2-[[3-(hydroxymethyl)indolin-1-yl]methyl]-6-methoxy-3H-quinazolin-4-one; and
6-chloro-2-(2,3-dihydro-1,4-benzoxazin-4-ylmethyl)-3H-quinazolin-4-one.

33. The compound according to claim 19, wherein said compound is selected from the group consisting of:
6-cyclopropyl-2-(indolin-1-ylmethyl)-3H-quinazolin-4-one;
2-[(8-fluoro-3,4-dihydro-2H-quinolin-1-yl)methyl]-6-methoxy-3H-quinazolin-4-one;
6-(dimethylamino)-2-[(3,3-dimethylindolin-1-yl)methyl]-3H-pyrido[3,4-d]pyrimidin-4-one;
2-[(3,3-dimethylindolin-1-yl)methyl]-7-fluoro-6-methoxy-3H-quinazolin-4-one;
7-chloro-2-[(3,3-dimethylindolin-1-yl)methyl]-6-methoxy-3H-quinazolin-4-one;
2-[(3,3-dimethylindolin-1-yl)methyl]-6-methoxy-3H-quinazolin-4-one;
6-methoxy-2-[(3-methylindolin-1-yl)methyl]-3H-quinazolin-4-one, enantiomer 1;
6-(dimethylamino)-2-[(3,3-dimethylindolin-1-yl)methyl]-3H-quinazolin-4-one;
2-[(3-isopropylindolin-1-yl)methyl]-6-methoxy-3H-quinazolin-4-one;
6-(dimethylamino)-7-fluoro-2-(indolin-1-ylmethyl)-3H-quinazolin-4-one;
6-(azetidin-1-yl)-2-(indolin-1-ylmethyl)-3H-quinazolin-4-one;
6-methoxy-2-[(3-methylindolin-1-yl)methyl]-3H-quinazolin-4-one;
2-(indolin-1-ylmethyl)-6-methoxy-3H-quinazolin-4-one;
6-(dimethylamino)-2-(indolin-1-ylmethyl)-3H-quinazolin-4-one;
2-(indolin-1-ylmethyl)-6-isopropyl-3H-quinazolin-4-one;
2-[(6-fluoroindolin-1-yl)methyl]-6-methoxy-3H-quinazolin-4-one;
6-methoxy-2-(2,3,4,5-tetrahydro-1-benzazepin-1-ylmethyl)-3H-quinazolin-4-one;
7-fluoro-2-(indolin-1-ylmethyl)-6-methoxy-3H-quinazolin-4-one;
6-chloro-2-[(3-methylindolin-1-yl)methyl]-3H-quinazolin-4-one enantiomer 1;
2-[(8-chloro-3,4-dihydro-2H-quinolin-1-yl)methyl]-6-methoxy-3H-quinazolin-4-one;
2-[(3,3-dimethylindolin-1-yl)methyl]-6-methoxy-3H-pyrido[3,4-d]pyrimidin-4-one;
2-(indolin-1-ylmethyl)-6-[3-(methoxymethyl)pyrrolidin-1-yl]-3H-quinazolin-4-one;
6-chloro-2-(2,3,4,5-tetrahydro-1-benzazepin-1-ylmethyl)-3H-quinazolin-4-one;
6-chloro-2-[(3,3-dimethylindolin-1-yl)methyl]-3H-quinazolin-4-one;
2-(indolin-1-ylmethyl)-6-pyrrolidin-1-yl-3H-quinazolin-4-one;
6-methoxy-2-[(1-methyl-3,4-dihydro-2H-1,5-benzodiazepin-5-yl)methyl]-3H-quinazolin-4-one;
6-chloro-2-[(5-methyl-3,4-dihydro-2H-quinolin-1-yl)methyl]-3H-quinazolin-4-one;
2-(indolin-1-ylmethyl)-6-methyl-3H-quinazolin-4-one;
2-[(6-methoxy-4-oxo-3H-quinazolin-2-yl)methyl]-3,4-dihydro-1H-isoquinoline-5-carbonitrile;
6-chloro-2-[(5-methoxy-3,4-dihydro-1H-isoquinolin-2-yl)methyl]-3H-quinazolin-4-one;
6-chloro-2-(1,2,3,4-tetrahydroisoquinolin-2-ium-2-ylmethyl)-3H-quinazolin-4-one chloride;
6-methoxy-2-[(5-methoxy-3,4-dihydro-1H-isoquinolin-2-yl)methyl]-3H-quinazolin-4-one;
6-chloro-2-[(7-methyl-3,4-dihydro-1H-isoquinolin-2-yl)methyl]-3H-quinazolin-4-one; and
6-chloro-2-[(3-methylindolin-1-yl)methyl]-3H-quinazolin-4-one enantiomer 2.

34. A pharmaceutical composition comprising a compound according to claim 19 and an acceptable pharmaceutical excipient.

35. A method of treating cancer comprising administering a compound according to claim 19, a compound selected from the group consisting of 6-chloro-2-(indolin-1-ylmethyl)-3H-quinazolin-4-one; 6-chloro-2-(3,4-dihydro-2H-quinolin-1-ylmethyl)-3H-quinazolin-4-one; 6-chloro-2-(1,2,3,4-tetrahydroisoquinolin-2-ium-2-ylmethyl)-3H-quinazolin-4-one; 6-chloro-2-[(4-hydroxy-5,8-dimethoxy-3,4-dihydro-1H-isoquinolin-2-yl)methyl]-3H-quinazolin-4-one; 2-(indolin-1-ylmethyl)-6,7-dimethoxy-3H-quinazolin-4-one; and 7-chloro-2-(indolin-1-ylmethyl)-3H-quinazolin-4-one, or a pharmaceutical composition comprising said compound to a subject having cancer.


SEQUENCE LISTING (4 sequences)

SEQ ID NO: 1 - DNA, 45 bp, ARTIFICIAL
  PRIMER
gatatcgaattcatgggcaaaggagatcctaagaagccgagaggc

SEQ ID NO: 2 - DNA, 44 bp, ARTIFICIAL
  PRIMER
tatcagctcgagacttcatcatcatcatcttcttcttcatcttc

SEQ ID NO: 3 - DNA, 49 bp, ARTIFICIAL
  PRIMER
gatatcgaattcatgctgctatccgtgccgctgctgctcggcctcctcg

SEQ ID NO: 4 - DNA, 48 bp, ARTIFICIAL
  PRIMER
tatcagctcgagaccagctcgtccttggcctggccggggacatcttcc